(19) United States
(12) Patent Application Publication
(10) Pub. No.: US 2026/0114883 A1
(43) Pub. Date: Apr. 30, 2026

(54) SYSTEMS AND METHODS FOR REMOVAL OF BIOLOGICAL OBJECTS FROM ANATOMICAL STRUCTURES WITHIN A BODY

(71) Applicant: Ventaris Surgical, Inc., San Carlos, CA (US)

(72) Inventors: Surag Mantri, East Palo Alto, CA (US); Scott Taylor, San Martin, CA (US); Peter Bentley, Reno, NV (US); Chris Danek, San Carlos, CA (US)

(21) Appl. No.: 19/373,586

(22) Filed: Oct. 29, 2025

Related U.S. Application Data
(60) Provisional application No. 63/714,760, filed on Oct. 31, 2024.
(60) Provisional application No. 63/797,867, filed on Apr. 30, 2025.

(51) Int. Cl. A61B 17/22; A61B 17/00; A61B 18/00; A61B 18/20; A61B 18/26; A61B 90/00

(52) U.S. Cl. CPC A61B 17/2202; A61B 18/26; A61B 2017/00084; A61B 2017/22079; A61B 2018/00511; A61B 2018/00744; A61B 2018/00791; A61B 2018/206; A61B 2090/064; A61B 2217/007; A61B 2218/002; A61B 2218/007

(57) ABSTRACT

Implementations of the present disclosure generally relate to medical aspiration devices and their methods of use. In some cases, the devices are configured to create a vacuum to capture and/or immobilize a solid deposit at a location internal to a subject. Such configurations may be useful, for example, for removing intact kidney stones small enough to pass through the ureter. In some cases, the devices further include an ablation instrument. Such devices may be useful, for example, for ablating larger solid deposits into a plurality of smaller particles at a location internal to the subject (e.g., ablating kidney stones that are too large to pass through the ureter). In some cases, the vacuum created by the devices is used to remove the plurality of smaller particles from the location internal to the subject.

### CROSS-REFERENCE TO RELATED APPLICATIONS

The present application claims priority to U.S. Provisional Patent Application No. 63/714,760, filed on Oct. 31, 2025, and U.S. Provisional Patent Application No. 63/797,867, filed on Apr. 30, 2025, each of which is incorporated by reference herein in its entirety for all purposes and forms a part of this specification. Any and all applications for which a foreign or domestic priority is identified in the Application Data Sheet as filed with the present application are hereby incorporated by reference under 37 CFR 1.57.

### TECHNICAL FIELD

The present disclosure generally relates to medical systems and related methods, such as minimally invasive surgical devices and systems for manipulating and removing objects from an anatomical structure.

### DESCRIPTION OF THE RELATED ART

Minimally invasive surgeries offer numerous advantages over traditional open surgical techniques. These encompass reduced surgical trauma, decreased recovery time, shorter hospital stays, and/or potentially a diminished risk of infection and other complications. However, certain challenges persist in minimally invasive surgery, notably the requirement for precise manipulation and removal of objects within the body while preventing damage to the adjacent tissues. This precision is of paramount importance in fluid-filled environments and/or intact organs, such as the kidney, gall bladder, urinary bladder, urinary tract, blood vessels, or other body cavities or organs.

For kidney stone treatment, the two primary approaches include ureteroscopic lithotripsy and percutaneous nephrolithotomy (PCNL), which incorporates its variant, the mini-PCNL. Both procedures, whether utilizing a ureteroscope or a nephroscope, generally encounter parallel challenges, including extended operation times, an obscured visual field, difficulties in extracting residual fragments, stone retropulsion, high intra-luminal pressure, challenges with temperature regulation, and/or instrument size that limit ureteral access. Accordingly, improved systems and methods are needed for safe and effective removal of biological objects.

### SUMMARY

Described herein are systems, devices and methods for removal of a biological object from an anatomical structure. Certain systems, devices and methods may be applied to removing a biological object such as a kidney stone from a urinary tract. Certain systems and methods described herein can use a Venturi-effect and/or a jet-pump effect with entrainment to attract the biological object towards the one or more openings.

A system for use in a removal procedure of a biological object from an anatomical structure can include: a catheter body including a proximal end and a distal end configured to be inserted into the anatomical structure, the catheter body including one or more openings configured to receive the biological object; an aspiration lumen positioned at least partially in the catheter body, the aspiration lumen being in fluid communication with the one or more openings, and the aspiration lumen being configured to aspirate and transport at least a portion of the biological object from the one or more openings toward a proximal end of the aspiration lumen; and a supply lumen configured to transport a liquid from a liquid source to the distal end of the catheter body, the supply lumen including a first portion extending at least between the proximal end and the distal end of the catheter body and a second portion at the distal end of the catheter body, and the second portion configured to eject the liquid outside the supply lumen and create vacuum via a jet pump effect to attract the biological object toward the one or more openings.

In some implementations, a longitudinal axis of the second portion can be oriented at an angle relative to a longitudinal axis of the first portion such that the liquid is ejected from the supply lumen at an angle relative to the longitudinal axis of the first portion greater than 90 degrees and less than 180 degrees.

In some implementations, the second portion can be configured to eject the liquid along a helical path within the aspiration lumen.

In some implementations, the system can include a plurality of vent port openings formed in the catheter body and fluidly connected to the aspiration lumen, wherein the plurality of vent port openings is configured to aspirate a portion of the biological object and/or is configured to resupply into the anatomical structure at least some of the liquid ejected from the supply lumen.

In some implementations, the aspiration lumen can be configured to be in fluid communication with a vacuum source. The vacuum source can be configured to supply vacuum at a first vacuum level to the proximal end of the aspiration lumen and to facilitate aspiration of the biological object from the one or more openings toward the proximal end of the aspiration lumen.

In some implementations, the system may cause ejection of liquid from the supply lumen into the catheter body to create a vacuum via entrainment.

In some implementations, at least one opening of the one or more openings can be positioned at a tip of the distal end of the catheter body.

In some implementations, at least one opening of the one or more openings can be positioned on a side of the catheter body.

In some implementations, the supply lumen can be positioned at least partially in the aspiration lumen.

In some implementations, the supply lumen can be positioned along an inner wall within the catheter body without obstructing a central passage within the catheter body.

In some implementations, the system can include an endoscope configured to support the catheter body.

In some implementations, the system can include an ablation device configured to be delivered through the aspiration lumen.

A method of removing a biological object from an anatomical structure can include: creating a vacuum with a catheter in the anatomical structure, the catheter including a proximal end and a distal end, an aspiration lumen positioned in a catheter body of the catheter, and a supply lumen configured to transport a liquid from a liquid source to the distal end of the catheter, wherein the vacuum is created at one or more openings in the catheter body to attract a biological object toward the one or more openings, the vacuum being created, via a jet pump effect, by transporting the liquid through the supply lumen and ejecting the liquid into the aspiration lumen; and aspirating at least a portion of the biological object through the aspiration lumen towards the proximal end of the aspiration lumen.

In some implementations, the supply lumen can include a bend at the distal end of the catheter, the bend being greater than 90 degrees and less than 180 degrees.

In some implementations, the biological object can be a urinary calculus, and the method can include fragmenting the urinary calculus by ablation and aspirating at least one fragment of the urinary calculus through the aspiration lumen toward the proximal end of the aspiration lumen.

In some implementations, the vacuum can create a vortex within the aspiration lumen adjacent to the one or more openings.

In some implementations, the vacuum may be created by ejecting the liquid from the supply lumen at a helical path within the catheter body.

In some implementations, the method can include regulating flow through the aspiration lumen.

In some implementations, the method can include applying negative pressure to the proximal end of the catheter to aspirate the portion of the biological object.

In some implementations, the method can include breaking the biological object into a plurality of fragments with ablation.

A method of removing a biological object from an anatomical structure can include: creating a vacuum with a catheter in the anatomical structure, the catheter including a proximal end and a distal end, an aspiration lumen positioned in a catheter body, and a supply lumen positioned in the aspiration lumen, the vacuum being created at one or more openings in the catheter body to attract a biological object toward the one or more openings by ejecting a liquid from an end of the supply lumen into the aspiration lumen; breaking the biological object captured and retained at the one or more openings in the catheter body into a plurality of fragments; aspirating at least some of the plurality of fragments of the biological object through the one or more openings in the catheter body and transporting at least some of the plurality of fragments of the biological object along the aspiration lumen toward the proximal end of the catheter; and aspirating through a plurality of vent port openings formed in the catheter body at least some of the plurality of fragments of the biological object.

In some implementations, the systems and methods disclosed herein may include a cup, a choke, and a working channel extension. In some implementations, the supply lumen is configured to eject the liquid outside the supply lumen, wherein ejecting the liquid forms a low-pressure zone within the system. In some implementations, a suction force is generated for attracting biological objects toward the aspiration lumen. In some implementations, a suction force between about 0.65 mN and 4.02 N is generated at the aspiration port opening. In some implementations, a flow rate through the aspiration port opening is the same as a flow rate through the vent port openings. In some implementations, the systems and methods disclosed herein include a reciprocator. In some implementations, the reciprocator includes a body portion and a rotating cam, wherein rotation of the cam axially moves a portion of the body portion. In some implementations, a movement of the reciprocator axially moves an ablation instrument positioned within the aspiration lumen. In some implementations, a body portion of the reciprocator includes a first body portion and a second body portion, wherein the first body portion is configured to move axially relative to the second body portion via rotation of the rotating cam.

Other implementations of the systems, devices and methods are described below and in the clauses and claims at the end of this specification.

### BRIEF DESCRIPTION OF THE DRAWINGS

Non-limiting examples of the present disclosure will be described by way of example with reference to the accompanying figures. For purposes of clarity, not every component is labeled in every figure, nor is every component of each example of the disclosure shown where illustration is not necessary to allow those of ordinary skill in the art to understand the disclosure.

FIG. 1 is a top view of a patient prepared for a ureteroscopy.

FIG. 2A is a top view of a ureteroscope being inserted into a patient.

FIG. 2B is a top view of a ureteroscope being positioned within a patient's kidney.

FIGS. 2C-2H are cross-sectional views of steps of removing a kidney stone from a patient's kidney.

FIG. 3A is a schematic diagram of a distal end of an example liquid jet aspiration device.

FIG. 3B is a schematic diagram of a distal end of an example liquid jet aspiration device.

FIG. 3C is a schematic diagram of an example liquid jet aspiration device demonstrating a Venturi principle effect.

FIG. 3D is a cross-sectional view of the example liquid jet aspiration device of FIG. 3A along line D-D.

FIG. 4A is a schematic diagram of a distal end of a liquid-jet powered medical instrument movable within a surrounding guide channel or sheath and suitable for use in a surgical procedure at a location internal to a subject.

FIG. 4B is an illustration of a liquid-jet powered medical instrument similar to that depicted in FIG. 4A illustrating (arrows) typical liquid ingress and egress patterns with the surrounding sheath removed.

FIG. 4C is an illustration of a liquid-jet powered medical instrument similar to that depicted in FIG. 4A illustrating typical liquid ingress and egress patterns with the surrounding sheath in a first axial position with respect to a distal tip of the instrument.

FIG. 4D is an illustration of a liquid-jet powered medical instrument similar to that depicted in FIG. 4A illustrating typical liquid ingress and egress patterns with the surrounding sheath in a second axial position with respect to a distal tip of the instrument.

FIG. 4E is an illustration of a liquid-jet powered medical instrument similar to that depicted in FIG. 4A illustrating typical liquid ingress and egress patterns with the surrounding sheath in a third axial position with respect to a distal tip of the instrument.

FIG. 5A is a schematic diagram of distal end of an example combination aspiration-ablation system.

FIGS. 5B and 5C are depictions of a combination aspiration-ablation system similar to that depicted schematically in FIG. 5A, but as deployed with a multi-lumen access catheter or access catheter containing a multi-lumen guide insert (as illustrated), having guide channels for the aspiration catheter (immobilizing a kidney stone as illustrated) and an ablation tool.

FIG. 6A is a schematic diagram of an example operational system for powering and controlling operation of a liquid-jet powered Venturi-assisted medical aspiration instrument for use at a within an organ or other location internal to a subject.

FIGS. 6B-6F are schematic diagrams of various specific and example implementations of the operational system for powering and controlling operation of a liquid-jet powered Venturi-assisted medical aspiration instrument shown in FIG. 6A.

FIG. 7 is a schematic diagram of a distal end of an example liquid-jet powered Venturi-assisted medical aspiration instrument with one or more aspiration port opening sized to retain and immobilize solid deposits of interest.

FIG. 8A is a schematic diagram of a distal end of an example liquid-jet powered Venturi-assisted medical aspiration instrument with at least one aspiration port opening.

FIGS. 8B-8C are schematic diagrams of example configurations for the aspiration port openings of the liquid-jet powered Venturi-assisted medical aspiration instrument of FIG. 8A, with and without a mesh for blocking entry of larger debris from passing through the aspiration port opening.

FIGS. 8D-8E are schematic diagrams of example configurations for the one or more vent port openings of an instrument such as depicted in FIG. 8A, with and without a mesh for blocking entry of larger debris from passing through the one or more vent port openings.

FIGS. 9A-9F show as (partially transparent to show internal detail) an illustration of a distal end of an example liquid-jet powered medical aspiration instrument with a plurality of circumferentially disposed aspiration ports, as well as several cross-sectional views of the instrument (FIGS. 9B-9D) and detailed views of an inverter plate (FIG. 9E) and an inverter cap (FIG. 9F) downstream of a high pressure liquid carrying tube and upstream of a liquid-jet-forming nozzle.

FIGS. 10A-10B are schematic diagrams of an example operation system for integrated sensing.

FIG. 11 is a schematic diagram of an example operation system with integrated sensing.

FIG. 12 is a schematic diagram of an example implementation for controlling motorized movement and automated manipulation of a solid deposit.

FIGS. 13A-13E are illustrations of various views of an ureteroscope including an aspiration-ablation system.

FIG. 14A-14C are illustrations of an ureteroscope having concentric lumens.

FIG. 15 is a schematic diagram of a ureteroscope system.

FIGS. 16A-16B are schematic diagrams of an example implementation of an evacuation tube including an aspiration-ablation system.

FIGS. 17A-17F are illustrations of example implementations of an evacuation tube with a pivoted J-tube bend.

FIG. 18 is an illustration of an example liquid supply lumen having a J-tube bend.

FIGS. 19A-19C illustrate side and front views of an example evacuation tube and a liquid supply lumen having a J-tube bend.

FIG. 20 illustrates a system using the evacuation tube and liquid supply lumen having a J-tube bend of FIGS. 19A-19B.

FIGS. 21A-21C illustrate a fluid flow graph of the system of FIG. 20.

FIGS. 22A-22C illustrate an example implementation of an evacuation tube with a plurality of pivoted J-tube bends.

FIGS. 23A-23C illustrate example implementations of sensors disposed at a distal end of a catheter, a sheath, and/or a ureteroscope.

FIGS. 24A-24C illustrate example implementations of an evacuation tube with a side-fire J-tube bend.

FIGS. 25A-25C illustrate example implementations of an evacuation tube with a plurality of pivoted J-tube bends.

FIGS. 26A-26B are cross-sectional perspective views of the distal tip of an example ureteroscope showing a side view of a fluid flow path with an ablation instrument positioned within the evacuation tube.

FIGS. 27A-27D are views of a reciprocator for axially moving an ablation instrument positioned within the evacuation tube.

### DETAILED DESCRIPTION

Implementations of the present disclosure generally relate to biological object removal systems, devices, and related methods. Certain implementations generally relate to devices and systems configured to generate a vacuum, for instance, capable of manipulating, immobilizing, and/or aspirating a solid object and/or debris from an ablated or comminuted solid object in a liquid filled environment. In some cases, the vacuum is generated and/or maintained via a Venturi effect and/or jet-pump effect with entrainment. Configurations described herein may be useful, for example, for capturing, manipulating, immobilizing, and/or removing biological objects from anatomical structures inside the body, such as intact kidney stones, or portions of kidney stones, or debris from ablated kidney stones. For example, some kidney stones may be generally small enough to pass through the ureter of a subject but may be unable to due to, for example, a disease state of the subject. In some cases, the kidney stones are too large or are otherwise unable to pass through the ureter of the subject. In some cases, the systems and devices described herein include an ablation device (also referred to as an ablation instrument), such as a laser or ultrasound, configured to ablate one or more solid objects into a plurality of smaller solid objects. Ultrasound can include an ultrasound ablation tool configured to provide ultrasonic lithotripsy. Such systems may be useful, for example, for breaking up kidney stones that are too large to pass through the ureter of a subject. Additionally, or alternatively, the vacuum created by the devices and systems described herein may be useful for aspirating a plurality of smaller solid objects into the device, thus removing it from the liquid filled environment (e.g., the kidney).

Some implementations generally relate to methods of using the systems and devices disclosed herein. For example, the devices and systems described herein may be useful for capturing and/or removing a biological object at a location internal to a subject (such as, from an anatomical structure). Biological objects can include blood clots, tumors, tissue samples, and urinary or fragmented urinary calculi such as bladder stones, ureter stones, and kidney stones.

The phrase “location internal to a subject” as used herein generally refers to a cavity, orifice, anatomical structure, or organ within a subject. For example, in some cases, the location internal to the subject is a kidney, a bladder, a heart, a colon, a duodenum, an ileum, a jejunum, a stomach, an esophagus, an intestine, a mouth, a liver, a lung, a pancreas, a spleen, a lymph node, a (blood) vessel, a gland, an ear canal, a urethra, a uterus, a gallbladder, an ovary, or a nasal cavity. In an example set of cases, the location internal to the subject is a kidney or bladder.

The term “subject,” as used herein, refers to an individual organism such as a human or an animal. In some cases, the subject is a mammal (e.g., a human, a non-human primate, or a non-human mammal), a vertebrate, a laboratory animal, a domesticated animal, an agricultural animal, or a companion animal. In some cases, the subject is a human. In some cases, the subject is a rodent, a mouse, a rat, a hamster, a rabbit, a dog, a cat, a cow, a goat, a sheep, or a pig.

In some cases, the articles and systems described herein are administered to a subject. In certain cases, the system may be administered surgically (e.g., inserted), through an incision, typically endoscopically using a catheter or similar, in other cases, the devices may be inserted into the body orally, rectally, vaginally, nasally, or uretherally. In certain cases, the system is administered such that at least a portion of the system accesses a location internal to the subject such as an organ (e.g., the kidney).

In some cases, the system is configured to manipulate a liquid at the location internal to the subject. As used herein, a “liquid” is given its ordinary meaning. A liquid generally cannot maintain a defined shape and will flow during an observable time frame to fill the container in which it is put. Thus, the liquid may have any suitable viscosity that permits flow. If two or more liquids are present, each liquid may be independently selected among essentially any liquids by those of ordinary skill in the art. Typically, the liquid will be sterile water or sterile normal saline or phosphate buffered saline or other osmotically balanced liquid or another IV fluid suitable for use in a human patient.

Implementations of the disclosure are related to devices and systems suitable for use in a surgical procedure at a location internal to a subject. In some cases, the devices are suitable for use at a location internal to a subject at least partially filled with a surrounding liquid (e.g., an organ of the subject).

In some cases, the devices described herein are liquid-jet powered instruments. In some cases, the instruments described herein are Venturi-assisted instruments. In some cases, the instruments described herein are useful for ablation and/or ablation assistance (e.g., of a solid deposit such as a kidney stone). For example, the instruments described herein may be useful for capturing a solid deposit such that it may be ablated (e.g., by an ablation instrument such as a laser, ultrasound probe, or other suitable ablation instruments). As described herein, the liquid-jet powered instruments, Venturi-assisted instruments, and/or other instruments can refer to a catheter, an aspiration catheter, and/or an evacuation catheter.

### Overview

Kidney stones affect approximately 10% of individuals in the United States, with nearly 470,000 surgeries performed annually to relieve symptoms and prevent complications. Current treatment modalities include extracorporeal shock wave lithotripsy (ESWL), ureteroscopy (URS), and percutaneous nephrolithotomy (PCNL). Of these, URS is the most prevalent, accounting for approximately two-thirds of all kidney stone surgeries due to its versatility in treating a broad range of stone sizes using lithotripsy devices to break up stones and baskets to remove fragments and small stones. URS procedures have been steadily increasing by about 15% annually since 2012.

Kidney stone procedures aim to maximize the removal of stones and fragments. Despite advancements in lithotripsy and ureteroscopy technology, stone clearance remains inconsistent, particularly for larger or more complex stones, with stone-free rates (defined as no stones remaining on follow-up CT scan imaging) often declining to 50% in these cases. Residual fragments can lead to symptoms and complications, including acute stone events, regrowth, and infection. Consequently, achieving complete stone clearance is critical to reducing reintervention rates and optimizing long-term outcomes.

There are still necessary improvements in the efficiency and safety of URS, including pressure and temperature management inside the kidney, maintaining a clear visual field, and preventing stone fragments from moving away from operative devices. Enhancing these aspects of URS aims to prevent injury and reduce procedure times.

Recent innovations, such as direct in-scope suction (DISS) and flexible and navigable sheaths (FANS) aim to improve stone-free rates and procedural efficiency, though these technologies have limitations. DISS uses the ureteroscope's small working channel to aspirate dust and small debris, which enhances visibility and helps regulate intrarenal pressure (IRP). However, the narrow diameter of the working channel restricts the removal of larger fragments and causes vacuum pressure loss at the distal end, reducing its ability to effectively clear debris and control retropulsion. This compromised visibility and resulting retropulsion increase the risk of unintended ablation of kidney walls and reduce procedural efficiency. Furthermore, the reduction in suction can lead to an increase in IRP which can have significant consequences (as described below).

Ureteral access sheaths (UAS), including vacuum-assisted FANS models, offer an alternative approach by combining suction with irrigation to maintain low IRP and reduce the “snow globe” effect caused by debris during lithotripsy. FANS provide additional maneuverability, allowing for navigation through renal calyces to suction larger fragments. To use FANS efficiently, high-pressure irrigation is required notably to remove fragments. However, manual control of irrigation and suction often causes kidney distension or collapse during procedures due to difficulties in balancing in and out flow and maintaining IRP. This complexity adds to the surgeon's workload, as they must also manage laser ablation time and wattage to prevent temperature spikes, further diverting their focus and decreasing procedural efficiency, while amplifying the risks associated with managing pressure and temperature

Limitations in current laser lithotripsy techniques during URS often lead to prolonged procedures and incomplete stone clearance. Surgeons may attempt mechanical capture and removal of residual fragments with stone retrieval tools, adding time and complexity and requiring coordination with support staff. Alternatively, “dusting” techniques may be employed to break stones into passable particles, though residual dust and small fragments can lead to acute stone events. Limited visibility, often caused by dust clouds and bubbles, hampers precise targeting which leads to residual stone fragments and requires increased irrigation, prolonging the procedure. To prevent thermal injury, surgeons may lower laser wattage or operate it intermittently, which also increases procedure times.

Effective IRP management is essential to prevent complications during ureteroscopy. Although elevated irrigation flow improves visibility during lasing, it can concurrently increase IRP, raising the risk of pyelovenous, pyelolymphatic, and pyelosinus backflow, which can lead to severe outcomes like urosepsis, systemic infection, and kidney damage. These risks not only increase the cost of the procedure due to prolonged hospital stay but also increase the risk of patient death. While UAS and FANS improve fluid outflow to help manage IRP, their use entails risks. Larger-diameter UAS, though effective at reducing IRP, increases the risk of ureteral injury, especially in patients with narrow ureters. One study found that nearly 50% of patients experience ureteral wall damage with UAS over 11Fr, with the risk rising for even larger sheaths. Although UAS and FANS technologies can control IRP to prevent overpressure and distension, they risk kidney collapse under vacuum suction, leading to bleeding and other complications.

Emerging pressure-sensing ureteroscopes offer real-time IRP monitoring but do not actively control it, leaving pressure regulation in the hands of the surgeon, who must monitor pressure and manually adjust irrigation or use aspiration to lower IRP. This reactive approach relies on the surgeon's ability to interpret and respond promptly to prevent complications.

Temperature management with lasers, particularly Holmium: YAG and Thulium fiber lasers, also presents challenges. Temperatures above 43° C. can cause tissue damage, necessitating techniques like increased or chilled irrigation and reduced laser activation or wattage, which often prolong procedures.

Despite advancements, the current ureteroscopic lithotripsy paradigm challenges surgeons' ability to see, effectively clear stones and fragments, and manage pressure and temperature to safely navigate and treat renal calculi in the upper urinary tract. Existing methods frequently leave fragments behind, resulting in complications for over 40% of patients and requiring re-treatment within a year for up to ⅓ of cases.

The systems and methods described herein aim to address many of these limitations, offering substantial improvements in efficacy, safety, and efficiency. This novel approach has the potential to reduce complications, decrease re-treatment rates, and set a new standard for kidney stone surgery.

### Removal of Biological Objects

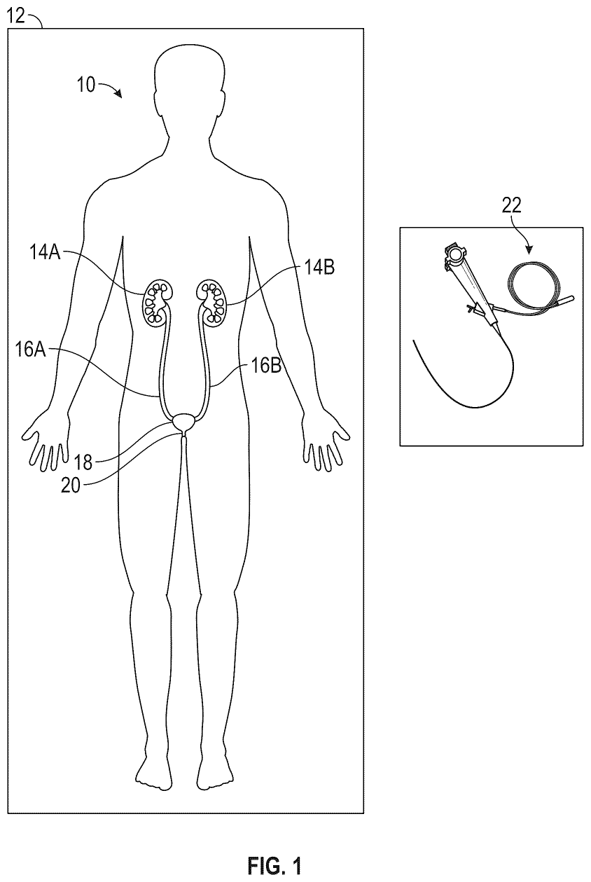

As shown in FIG. 1, a patient 10 can include a first kidney 14A and a second kidney 14B. The patient 10 can further include a first ureter 16A, a second ureter 16B, a bladder 18, and a urethra 20.

The first kidney 14A and the second kidney 14B can be internal organs located within the torso of the patient 10 and positioned below or inferior to the ribcage and laterally on either side of the patient's spine. The first kidney 14A and the second kidney 14B can be configured to process fluids. For example, the first kidney 14A and the second kidney 14B can filter blood, remove waste and extra fluid, balance fluids, and produce hormones and red blood cells. The first kidney 14A and the second kidney 14B can filter fluids resulting in urine. In some cases, the first kidney 14A and/or the second kidney 14B can produce solid deposits or kidney stones. The solid deposits can be formed when the fluids within the first kidney 14A and/or the second kidney 14B include too many crystal-forming substances in relation to the amount of fluid. In some examples, the first kidney 14A and/or the second kidney 14B can include a high concentration of calcium, oxalate, and/or uric acid in relation to the amount of fluid. Accordingly, the fluid is insufficient to dilute the crystal-forming substances and is insufficient to prevent the crystal-forming substances from forming solid deposits.

The first ureter 16A and the second ureter 16B can each be a hollow tube configured to transport fluids. The first ureter 16A and the second ureter 16B can be located in the patient's torso or abdomen.

The bladder 18 can be a hollow, elastic internal organ located in the lower part of a patient's abdomen. The bladder 18 can be configured to collect and store fluids. For example, the bladder 18 can be configured to collect and store urine from the first kidney 14A and/or the second kidney 14B.

The urethra 20 can be a hollow tube configured to transport fluids. The urethra can be in fluid communication with an external environment. Accordingly, the urethra 20 can be configured to expel fluids from the patient's body.

The first kidney 14A and the second kidney 14B can be in fluid communication with the bladder 18 via a corresponding first ureter 16A or second ureter 16B. For example, the first ureter 16A can extend between the first kidney 14A and the bladder 18 and the second ureter 16B can extend between the second kidney 14B and the bladder 18. Accordingly, fluids can be passed from the first kidney 14A and/or the second kidney 14B to the bladder 18 via the corresponding first ureter 16A or second ureter 16B. The bladder can be in fluid communication with an external environment via the urethra 20.

The above identified organs can define the urinary tract or urinary system of a patient. In a healthy patient, fluids such as urine can pass unobstructed through the urinary system. Solid deposits formed within the kidneys can block fluid flow within the urinary system. Blocking the fluid flow can result in a buildup of fluid and pressure causing pain and/or discomfort to the patient. Additionally, in some cases, the solid deposits can have an irregular shape. For example, the solid deposits can have sharp edges. The sharp edges can puncture tissue resulting in additional pain, discomfort, bleeding, and possible infection. To alleviate pain, discomfort, and/or otherwise treat or prevent further risk of injury, methods have been developed to remove solid deposits.

The devices and methods for removing solid deposits from a patient's urinary system can include an operating table 12 and instruments (i.e., devices) including an evacuation tube such as a ureteroscope 22.

The operating table 12 can be a device configured to support and/or restrain a patient during a medical procedure. For example, the operating table 12 may support an unconscious and/or medicated patient 10 for the duration of a medical procedure such as a ureteroscopy.

The ureteroscope 22 can include a thin, tube-shaped shaft. The thin, tube-shaped shaft can have an interior lumen sized to receive at least a portion of a solid deposit. The ureteroscope 22 can be configured to pass through the patient's urinary system to reach the solid deposit (such as, a kidney stone). The ureteroscope 22 can further include a light and a lens to assist a physician in navigating the patient's urinary system and/or identifying solid deposits. The ureteroscope 22 can include a working channel. In some cases, a laser can be placed into the working channel of the ureteroscope 22. The laser can be configured to apply energy to and break up solid deposits. Additionally or alternatively, in some cases, a gripping mechanism can be placed into the working channel of the ureteroscope 22. The gripping mechanism can be configured to secure one or more solid deposits for extraction.

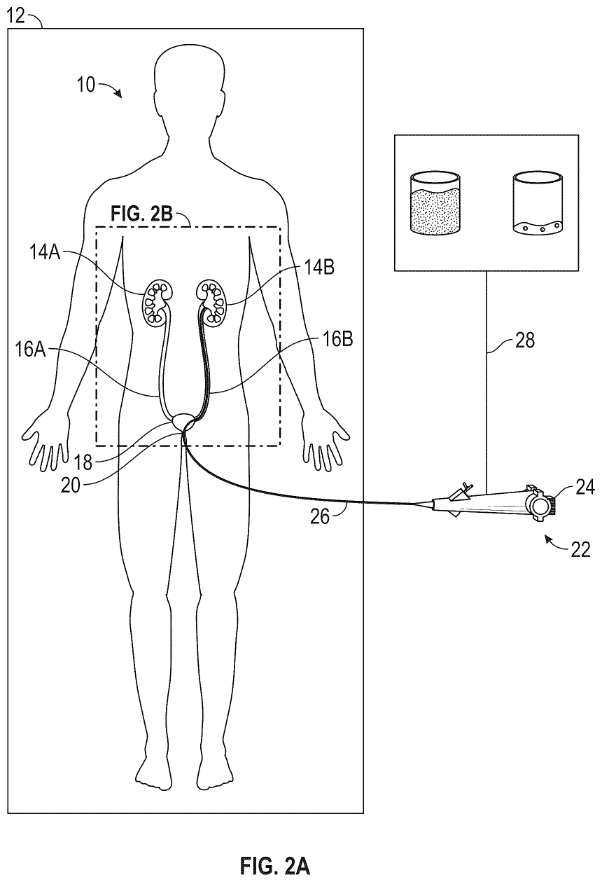

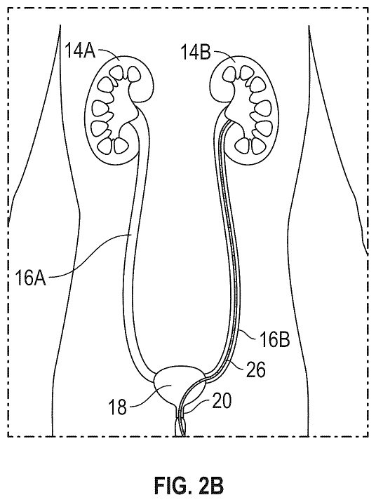

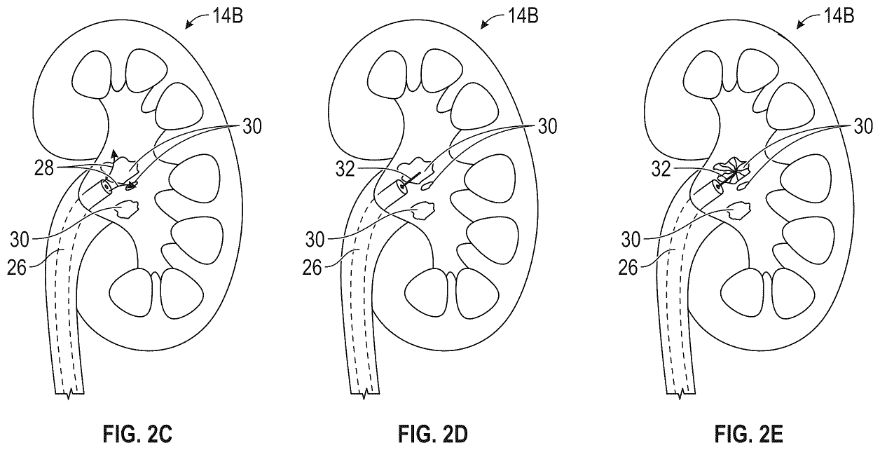

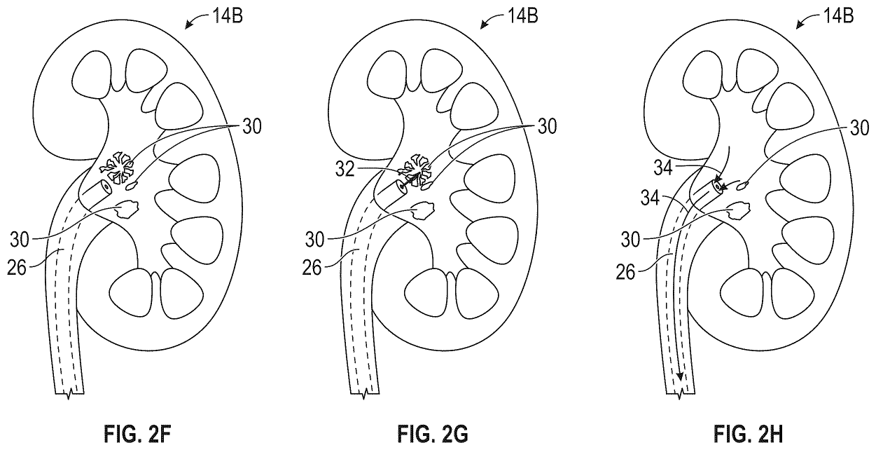

FIGS. 2A-2H illustrate methods and steps of using a ureteroscope 22 to remove solid deposits from a patient's kidney. While FIGS. 2A-2H illustrate the ureteroscope 22 being applied to the second kidney 14B, the approaches described herein can be utilized for removing solid deposits from either kidney and/or anywhere along the urinary system or, more generally, from any location in the body.

As shown in FIG. 2A, a patient 10 can be supported on an operating table 12. In some cases, the patient 10 can be medicated. For example, the patient 10 can be generally and/or locally anesthetized. The ureteroscope 22 can be introduced to the patient's urinary system. For example, the ureteroscope 22 can be introduced via the urethra 20. In some cases, the ureteroscope 22 can include a sheath 26 sized to pass through the patient's urethra 20, bladder 18, ureter 16A/16B and/or kidney 14A/14B. The sheath 26 can be a tube. For example, the sheath 26 can have an outer diameter between about 0.5 and 4 mm, or between about 1.5 and 3.5 mm. In some cases, the sheath 26 can have a length between 40 cm and 100 cm. In some cases, the sheath 26 can have a length between 50 cm and 70 cm. For example, the sheath 26 can be about 60 cm in length. The sheath 26 can include the working channel of the ureteroscope 22. The working channel can extend through the length of the sheath 26. The working channel can have an outer diameter smaller than the outer diameter than the sheath 26. In some cases, the working channel can have an outer diameter between about 1.2 mm and 1.5 mm. The ureteroscope 22 can further include a handle portion 24 and a liquid supply catheter 28 (also referred to as a fluid supply catheter). The handle portion 24 can include an eyepiece or camera for a physician to see for navigation and identification of solid deposits. For example, the handle portion 24 can include a digital complementary metal oxide semiconductor (“CMOS”) camera. The handle portion 24 can be a Y-connector configured to fluidly connect the ureteroscope 22 to a first liquid supply (also referred to as a first liquid source, a first fluid supply, a first fluid source and/or a first fluid reservoir) for irrigation, a second liquid supply (also referred to as a second liquid source, a second fluid supply, a second fluid source and/or a second fluid reservoir) for aspiration, and/or laser or gripping mechanism insertion.

FIG. 2B illustrates a sheath 26 extending through the patient's urinary system and positioned in the second kidney 14B via the second ureter 16B, bladder 18, and urethra 18. The ureteroscope 22 may be generally used to identify and remove solid deposits.

FIG. 2C illustrates a step of inflating the second kidney 14B. As described herein, the ureteroscope 22 can include a sheath 26. The sheath 26 can be in fluid communication with a liquid source. The liquid source can also be referred to as a liquid supply and/or a liquid reservoir. The liquid source can be an input supply of a liquid to be used by a ureteroscope 22 to irrigate and/or flush the patient's urinary system. In some cases, the sheath 26 can introduce a liquid 28 into the second kidney 14B to expand the kidney and enhance visibility. For example, expanding the second kidney 14B can assist a physician in identifying one or more solid deposits 30.

In some cases, the ureteroscope 22 can include an end-effector located at the distal end of the sheath 26. The end-effector can be configured to grasp and secure a solid deposit. After securing the solid deposit, the sheath 26 and/or ureteroscope 22 can be removed from the patient 10. In some cases, the ureteroscope 22 can be in fluid communication with an aspiration source. For example, the ureteroscope 22 can be in fluid communication with a vacuum source. Accordingly, the ureteroscope 22 can be configured to aspirate solid deposits through the sheath 26. In some cases, the solid deposits may be too large to be removed by the ureteroscope 22.

FIG. 2D illustrates a step of applying energy 32 to at least one of the one or more solid deposits 30. In some cases, the energy 32 can be a laser. For example, a laser fiber can extend through the sheath 26 and be configured to emit laser energy toward at least one of the one or more solid deposits 30.

As shown in FIG. 2E-2G, the energy 32 can be sufficient to fracture and/or break up the solid deposits 30 into debris of fragmented solid deposits. The fragmented solid deposits can be smaller than the original solid deposit. For example, the fragmented solid deposits can be reduced to dust particles. Accordingly, the fragments may be sized sufficiently small to be aspirated through the sheath 26. In some cases, the fragments may remain too large to be aspirated through the sheath 26. Accordingly, the energy 32 can be applied to the fragments to further fracture and/or break up the solid deposits 30 as shown in FIG. 2G.

FIG. 2H illustrates a step of aspirating the solid deposits through the sheath 26. The ureteroscope 22 can be withdrawn and removed from the patient 10 after the solid deposits are successfully removed from the patient 10.

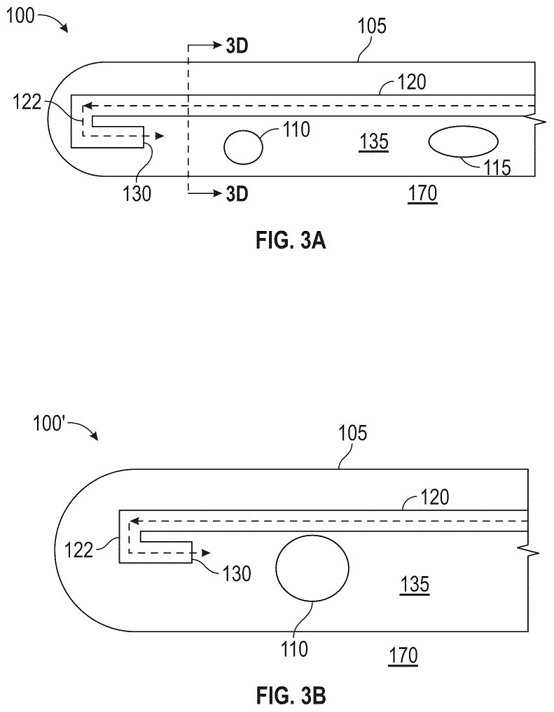

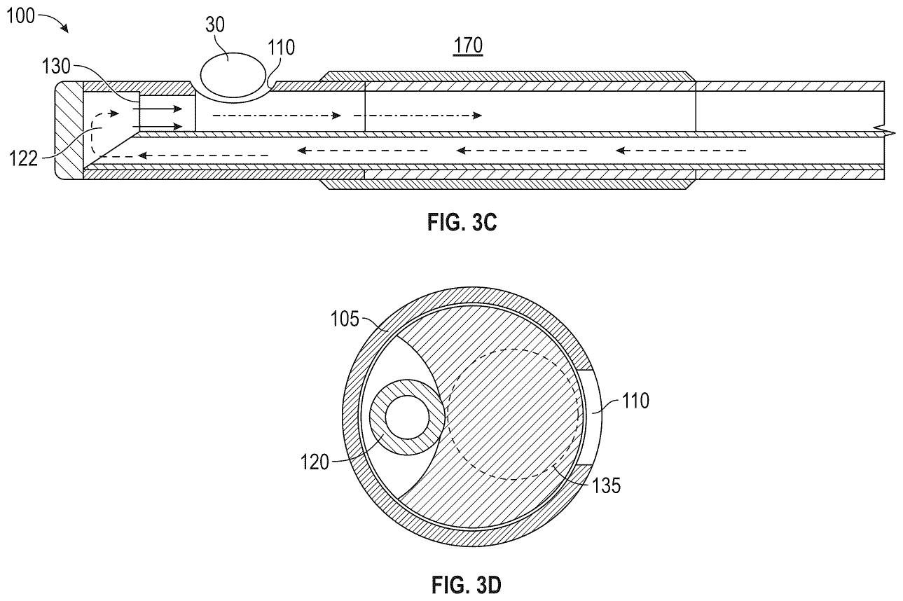

In some implementations, an instrument 100 (sometimes referred to as devices) described herein can include a liquid-jet powered aspiration tool or catheter. Such a device will typically include an evacuation tube. For example, as illustrated in FIG. 3A (which depicts the distal end of a liquid-jet powered aspiration catheter), instrument 100 can include an evacuation tube 105. In some cases, the instrument 100 can be the same as the ureteroscope 22 described herein. In some cases, the evacuation tube 105 can include one or more aspiration port openings 110 and the one or more vent port openings 115. In some cases, the one or more aspiration port openings 110 are formed on a sidewall of the evacuation tube 105. In some cases, the one or more vent port openings 115 is positioned downstream (e.g., relative to the flow of liquid within the evacuation tube 105, when the device is in operation) of the one or more aspiration port openings 110, as described in more detail herein and shown in FIG. 3A. Those of ordinary skill in the art would understand, based upon the teachings of this specification, that the instrument 100 is generally considered in operation when a liquid is flowing within one or more components of the instrument 100 (e.g., the evacuation tube, a liquid supply lumen, etc.).

As described herein, the instrument 100 can be configured, in some cases, to remove a biological object from a location internal to a subject (e.g., via ablation of the biological object such as a solid deposit and aspiration of at least a portion of the biological object from the location internal to the subject).

In some cases, the biological object can be a urinary calculus. For example, in some cases, the biological object can be a solid deposit or stone that forms in an anatomical structure such as the urinary tract. For example, the biological object can be formed in the kidneys, ureter, bladder, prostate gland, and/or urethra. In some cases, the stone is a kidney stone, a bladder stone, and/or a ureter stone. In some cases, the stone can be urinary (i.e., non-fragmented) or fragmented. A fragmented urinary calculus can include at least a portion of the urinary calculus. In some cases, the anatomical structure can include another specific, identifiable part of a patient's body. For example, the anatomical structure can include the gallbladder, salivary glands, and pancreas.

While FIG. 3A shows a single one of the one or more aspiration port openings 110 and a single one of the one or more vent port openings 115, the instrument 100 may include any suitable number of aspiration port openings 110 (e.g., one or more, two or more, three or more, four or more, etc. aspiration port openings 110) and/or any suitable number of vent port openings 115 (e.g., e.g., one or more, two or more, three or more, four or more, etc. vent port openings 115). In some cases, instrument 100 can include a liquid supply lumen 120.

While FIG. 3A shows the one or more aspiration port openings 110 and the one or more vent port openings 115 on the same side of the evacuation tube 105, in some cases, the one or more aspiration port openings 110 and the one or more vent port openings 115 may be located on different sides of the evacuation tube 105, as described in more detail herein.

In some cases, the evacuation tube 105 can include a liquid supply lumen 120. In some cases, the liquid supply lumen 120 can include an outlet 130. In some cases, the liquid supply lumen 120 can include a nozzle suitable for forming a liquid jet at the outlet 130. The liquid jet can have a flow rate between 20 and 60 ml/min. In some cases, the liquid jet can have a flow rate of about 40 ml/min. In some cases, the outlet 130 may be configured and positioned to direct a liquid jet (e.g., a liquid jet formed at a nozzle associated with the outlet 130) e.g., into the evacuation tube 105 (e.g., along an evacuation lumen 135 of the evacuation tube 105). In some cases, upon exiting the outlet 130 (and/or a nozzle positioned at the outlet 130), the liquid 122 flows past at least a portion of the one or more aspiration port openings 110 thereby generating a vacuum at the one or more aspiration port openings 110, relative to an environment external to the evacuation tube 105 e.g., while the instrument 100 is in operation. In some cases, the vacuum generated is sufficient to aspirate at least a portion of the surrounding liquid 170 from an environment external to the evacuation tube 105 (e.g., at a location internal to a subject or patient, as described in more detail herein). In some cases, the liquid jet, at the one or more aspiration port openings 110, generates a Venturi-created and/or Venturi-assisted vacuum while the instrument 100 is in operation, (e.g., sufficient for capturing and/or immobilizing and/or evacuating a solid deposit e.g., kidney stone, bladder stone, gall stone, etc., at a location internal to a subject). Nozzles suitable for use with the articles and systems are described in more detail, below. Taking advantage of the Venturi-effect (as explained herein) at or near the distal end of the ureteroscope to produce negative pressure can overcome the limitations of existing ureteroscopes that only include an aspiration pump at the proximal end. Higher suction can improve surgical efficacy by maintaining the solid deposits at an optimal distance from an ablation instrument thereby reducing possible retropulsion. Additionally, improved suction may position solid deposits away from organ tissue thereby reducing the risk of patient movement and accidental tissue ablation.

In some cases, the one or more vent port openings 115 may be sized, positioned and configured to eject at least a portion of the liquid 122 flowing along the evacuation lumen 135 under at least some conditions of normal operation, as described in more detail herein. The evacuation lumen 135 may comprise any suitable cross-sectional shape. Non-limiting examples of suitable cross-sectional shapes include triangles, squares, rectangles (e.g., having any suitable aspect ratio) circles, ovals, polygons (e.g., pentagons, hexagons, heptagons, octagons, nonagons, dodecagons, or the like), rings, irregular shapes, or the like. In some cases, the instrument 100 disclosed herein comprise a liquid source. Any suitable liquid source capable of being pressurized and passed through the devices disclosed herein may be used. For example, in some cases, the liquid source is a physiological fluid. In some cases, the physiological fluid is a saline solution (e.g., 0.9% wt NaCl), dextrose solutions (e.g., 5% wt), lactated ringers solution, ringers solution, dextran solution, plasmalyte solution or the like. Other solutions are also possible, according to other cases.

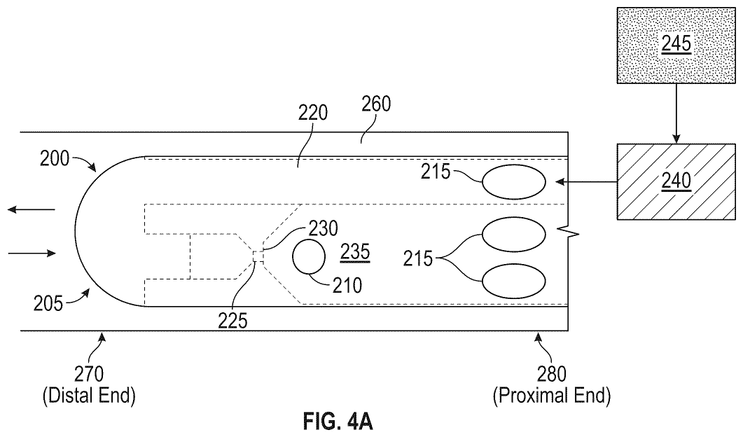

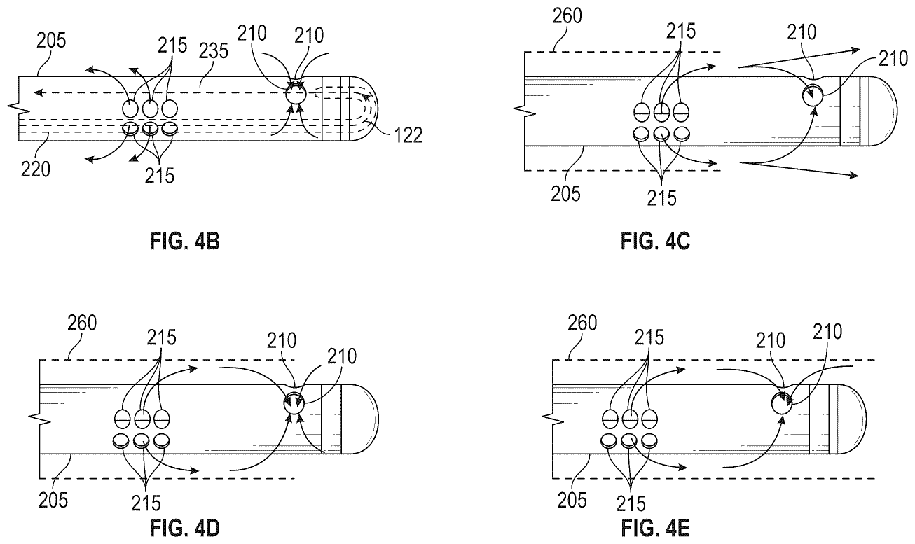

As would be understood by those of ordinary skill in the art, based upon the teachings of the specification, the one or more aspiration port openings 110 and/or the one or more vent port openings 115 can be configured and designed such that an internal portion (i.e., lumen) of the evacuation tube 105 is in fluidic communication with a surrounding liquid 170 (e.g., during operation of the instrument). In some cases, the one or more vent port openings 115 may be configured and designed such that at least a portion of the surrounding liquid 170 enters the evacuation tube 105 via the one or more vent port openings 115, when the instrument 100 is in operation. In some cases, the aspiration port openings 110 may be configured and designed such that at least a portion of a liquid 122 present and/or flowing within the evacuation tube 105 exits the evacuation tube 105 via the aspiration port openings 110 (e.g., to a surrounding environment at the location internal to the subject) during operation of the instrument 100. In some cases, liquid may not substantially pass through one or more aspiration port openings 110 when the instrument 100 is in operation. See FIG. 4B and associated discussion herein for more details regarding flow pattens and operational modes related to the aspiration and vent ports.

While FIG. 3A shows an instrument 100 including the one or more vent port openings 115, in some cases, the instrument 100 may not include any of the one or more vent port openings 115, (only aspiration port openings 110) as shown in FIG. 3B and as described in more detail below.

The instrument 100 may be configured as a liquid-jet powered aspiration medical instrument. For example, as illustrated in FIG. 4A, the instrument 200 (which can be similar to the instrument 100 or any other instrument described herein except for the differences described herein) can include an evacuation tube 205. In some cases, the evacuation tube 205 may be sized with respect to the size of the one or more aspiration port openings 210 such that a biological object (e.g., solid deposit such as a kidney stone, at least a portion of an ablated kidney stone, etc.) may be aspirated through the aspiration port opening(s) 210 and removed via the evacuation tube 205 (see e.g., FIG. 3D which depicts the relative size of an evacuation tube 105 and the one or more aspiration port openings 110).

In some cases, the evacuation tube 205 can include one or more aspiration port openings 210 e.g., formed in a side wall of the evacuation tube. In some cases, evacuation tube 205 can include one or more vent port openings 215. The one or more vent port openings 215 can be positioned downstream (e.g., when the instrument is in operation) of one or more aspiration port openings 210. In some cases, the instrument includes a liquid supply lumen 220. As shown in FIG. 4A, the liquid supply lumen 220 can be positioned against a sidewall of the evacuation tube 205. In some cases, the liquid supply lumen 220 includes a nozzle 225. The nozzle 225 can be configured to form a liquid jet at an outlet 230 of the liquid supply lumen 220. In some cases, the outlet 230 of the liquid supply lumen 220 may be configured and positioned to direct the liquid jet formed by the nozzle 225 into and along an evacuation lumen 235 of the evacuation tube 205. As shown in FIG. 4A, the evacuation lumen 235 can extend from a sidewall of the evacuation tube 205 opposite the liquid supply lumen 220 up to the medial sidewall of the liquid supply lumen 220. In some cases, one or more aspiration port openings 210 is positioned downstream relative to the outlet 230 when the instrument 200 is in operation (e.g., relative to the flow of liquid within the evacuation tube 205, when the instrument 200 is in operation). As described herein, the instrument 200 may include any suitable number of aspiration port openings 210 (e.g., one or more aspiration port openings 210, two or more aspiration port openings 210, three or more aspiration port openings 210, four or more aspiration port openings 210, five or more aspiration port openings 210, ten or more aspiration port openings 210, or any suitable plurality of aspiration port openings 210, e.g., distributed circumferentially around the evacuation tube).

In some cases, one or more vent port openings 215 may be positioned on a different side of the evacuation tube 205 relative to at least another one of the one or more vent port openings 215 and may be positioned downstream relative to the one or more aspiration port openings 210 when the instrument 200 is in operation. For example, in some cases, a first vent port opening 215 may be positioned on a different side of the evacuation tube 205, relative to a second vent port opening 215. As described herein, the instrument 200 may include any suitable number of vent port openings 215 (e.g., one or more vent port openings 215, two or more vent port openings 215, three or more vent port openings 215, four or more vent port openings 215, five or more vent port openings 215, ten or more vent port openings 215, or any suitable plurality of vent port openings 215).

In some cases, the number of aspiration port openings 210 and vent port openings 215 may include any suitable combination of each (e.g., one or more aspiration port openings 210 and one or more vent port openings 215, as described herein).

In some cases, the positioning of the aspiration port openings 210 relative to the vent port openings 215 can be such that a liquid (e.g., liquid contained within the liquid source 240), upon exiting the nozzle, flows past at least a portion of one or more aspiration port openings 210 generating a vacuum at the one or more aspiration port openings 210, relative to an environment external to the evacuation lumen 235, when the instrument 200 is in operation. As described in more detail herein, in some cases, the flow of the liquid past the one or more aspiration port openings 210 generates Venturi-effect suction, thereby producing the above-mentioned vacuum at the one or more aspiration port openings 210.

For example, as shown illustratively in FIG. 3C, and without wishing to be bound by theory, the flow of a liquid within a liquid supply lumen 120 (e.g., pressure, flow rate) may be altered, as may be the design of the nozzle, such that a desired level of Venturi-effect suction is produced proximate at least one of the one or more aspiration port openings 110.

Without wishing to be bound by any particular theory, the Venturi effect can explain a reduction in fluid pressure resulting from an increase in fluid flow of a fluid flowing through a constricted section. The Venturi effect can adhere to a conservation of flow rates relative to pressure and velocity. For example, the Venturi effect can follow Bernoulli's principle relating pressure and kinetic energy density. For example, the Venturi effect can follow the relationship P₁+½ ρ V₁²=P₂+½ ρ V₂² wherein P₁ is the pressure at the first location along a flow path, V₁ is the velocity of the fluid at the first location along the flow path, P₂ is the pressure at a second location along the flow path, and V₂ is the velocity of the fluid at the second location along the flow path, p is the fluid density assumed constant, wherein the first location and the second location are at the same height. Accordingly, the velocity and pressure are inversely related. For example, the pressure decreases proportional to the square of the velocity increase and vice versa. P₂=P₁+½ ρ (V₁²−V₂²). Based on this relationship as fluid velocities increase, negative pressure (suction) can be created.

A fluid flow rate can also be defined as the velocity of the fluid passing through an area. For example, a fluid flow rate can follow the relationship: AV, wherein A is the cross-sectional area of a tube, conduit, or other channel, and V is the velocity of the fluid passing through the cross-sectional area. The flow rate within an internal passageway can be constant for non-compressible fluids. Thus, the following relationship can apply for a constant flow rate: A₁V₁=A₂V₂, wherein A₁ is the cross-sectional area at a first location along a flow path, V₁ is the velocity of the fluid at the first location along the flow path, A₂ is the cross-sectional area at a second location along the flow path, and V₂ is the velocity of the fluid at the second location along the flow path. Accordingly, the velocity and area are inversely related. For example, the velocity can increase as the cross-sectional area decreases. Thus, the Venturi effect can be related to the constant flow rate such that the change in pressure is proportionally related to the square of the change in cross-sectional area. Accordingly, the pressure can decrease as the cross-sectional area decreases and vice versa. Thus, a nozzle or constriction in the fluid flow path can increase the velocity of the fluid flow and decrease pressure creating a vacuum.

In some cases, the instrument 100, device, and/or system may not include one or more vent port openings 115. For example, referring again to FIG. 3B, the instrument 100′ can include one or more aspiration port openings 110 but does not include one or more vent port openings 115. The instrument 100′ can be substantially similar to the instrument 100 described herein. In some cases, the instrument 100′ may be a modified version of the instrument 100. For example, the instrument 100′ may not include one or more vent port openings 115.

FIG. 3D illustrates a cross-sectional view of the evacuation tube 105. The aspiration catheters described herein may have any suitable maximum outer diameter and cross-sectional dimension. For example, in some cases, the evacuation catheter can be sized for administration to a subject (e.g., via a ureter or endoscopically via conventionally sized trocars or access catheters). In some cases, the evacuation tube 105 can have an outer diameter greater than or equal to 1 French (Fr), greater than or equal to 2 Fr, greater than or equal to 3 Fr, greater than or equal to 3.6 Fr, greater than or equal to 4 Fr, greater than or equal to 5 Fr, greater than or equal to 6 Fr, greater than or equal to 7, Fr, greater than or equal to 8 Fr, greater than or equal to 9 Fr, greater than or equal to 10 Fr, greater than or equal to 12 Fr, greater than or equal to 14 Fr, greater than or equal to 16 Fr, greater than or equal to 18 Fr, or 20 Fr. In some cases, the evacuation tube has an outer diameter less than or equal to 25 Fr, less than or equal to 18 Fr, less than or equal to 16 Fr, less than or equal to 14 Fr, less than or equal to 12 Fr, less than or equal to 10 Fr, less than or equal to 9 Fr, less than or equal to 8 Fr, less than or equal to 7 Fr, less than or equal to 6 Fr, less than or equal to 5 Fr, less than or equal to 4 Fr, less than or equal to 3.6 Fr, less than or equal to 3 Fr, or less than or equal to 2 Fr. In an example set of cases, the evacuation tube has an outer diameter of greater than or equal to 2 Fr and less than or equal to 4 Fr. Other combinations of ranges are also possible (e.g., greater than or equal to 1 Fr and less than or equal to 20 Fr). Other ranges are also possible.

Turning to FIGS. 4A-4E, the instrument 200 may include a liquid-jet forming aspiration catheter. In some cases, the aspiration catheter can include the evacuation tube 205 including one or more aspiration port openings 210 formed in the side wall of the evacuation tube. In some cases, the evacuation tube 205 can include the liquid supply lumen 220 including the nozzle 225. In some cases, the nozzle 225 may be suitable for forming a liquid jet at the outlet 230 of the liquid supply lumen 220. In some cases, the outlet 230 may be configured and positioned to direct the liquid jet formed by the nozzle 225 into and along the evacuation lumen 235 of the evacuation tube 205. The instrument 200 can include one or more aspiration port openings 210 formed in a side wall of evacuation tube 205. In some cases, the one or more aspiration port openings 210 may be positioned downstream relative to the outlet 230 and/or the nozzle 225. In some cases, the one or more aspiration port openings 210 may be configured and positioned at or near a distal end of the evacuation lumen 235. In some cases, during operation of the instrument 100, a liquid exiting the nozzle 225 can flow past at least a portion of the one or more aspiration port openings 210. Without wishing to be bound by any particular theory, it is believed that such configurations permit creation of a vacuum (e.g., via the Venturi-effect) at the one or more aspiration port openings 210, relative to an environment external to evacuation lumen 235. For example, the pressure within the evacuation lumen 235 at the one or more aspiration port openings 210 may be less than the pressure exterior to the evacuation tube 205. Accordingly, fluid can flow into the one or more aspiration port openings 210 in an attempt to equalize or balance the respective pressures. In some cases, the fluid flow can carry solid deposits along with the fluid thereby aspirating the solid deposits into the evacuation tube 205.

In some cases, the instrument 200 may include one or more vent port openings 215, for example, one or more vent port openings 215 including holes in a sidewall of the evacuation tube 205 providing fluidic communication with the evacuation lumen 235. The one or more vent port openings 215 can be configured and positioned closer a proximal end of the evacuation lumen 235 than the aspiration port openings 210. Those of ordinary skill in the art would understand, based upon the teachings of the specification, that the one or more vent port openings 215 may be configured and positioned at any suitable location of the instrument 200.

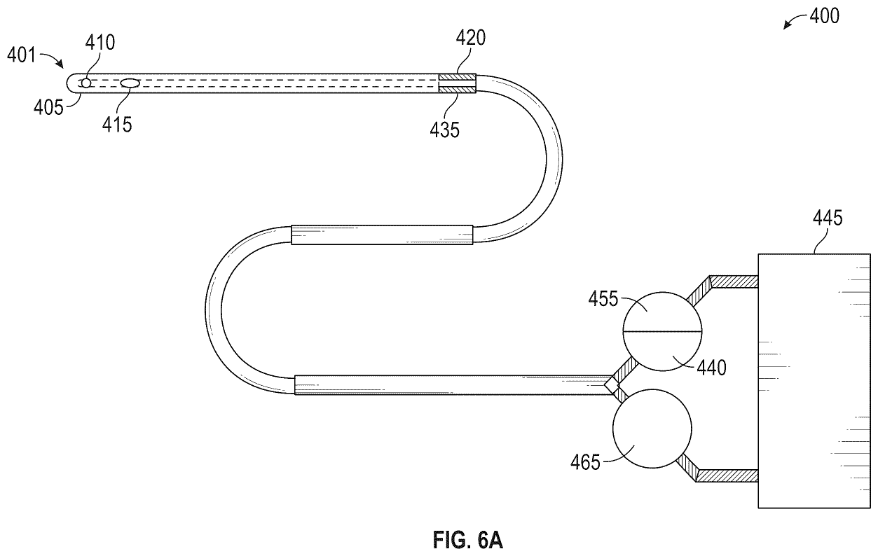

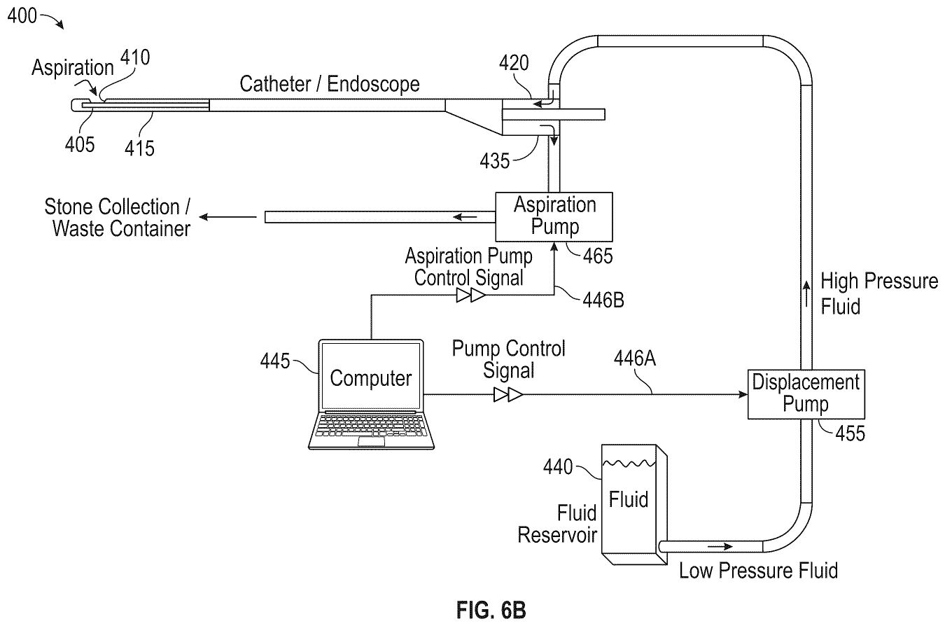

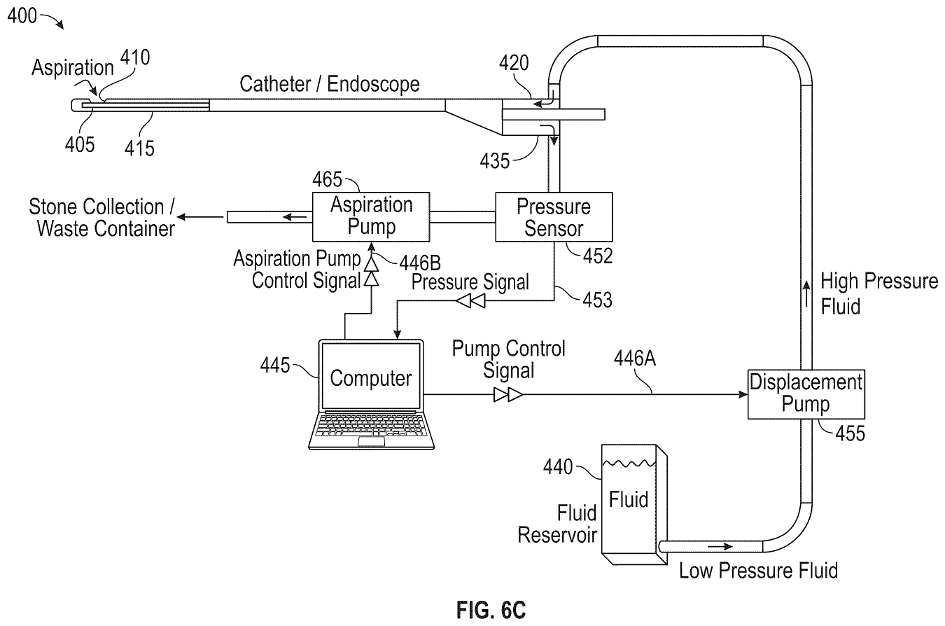

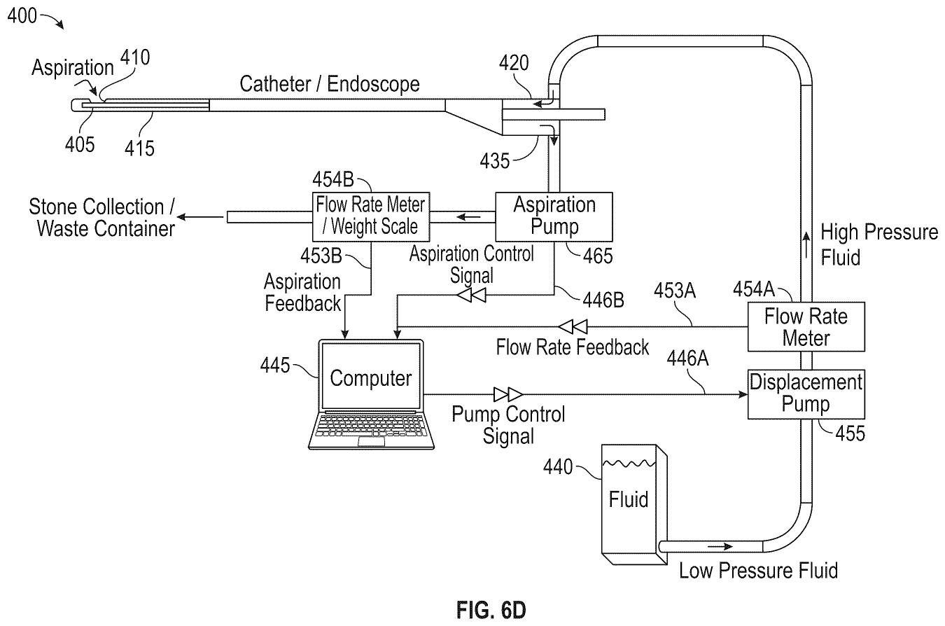

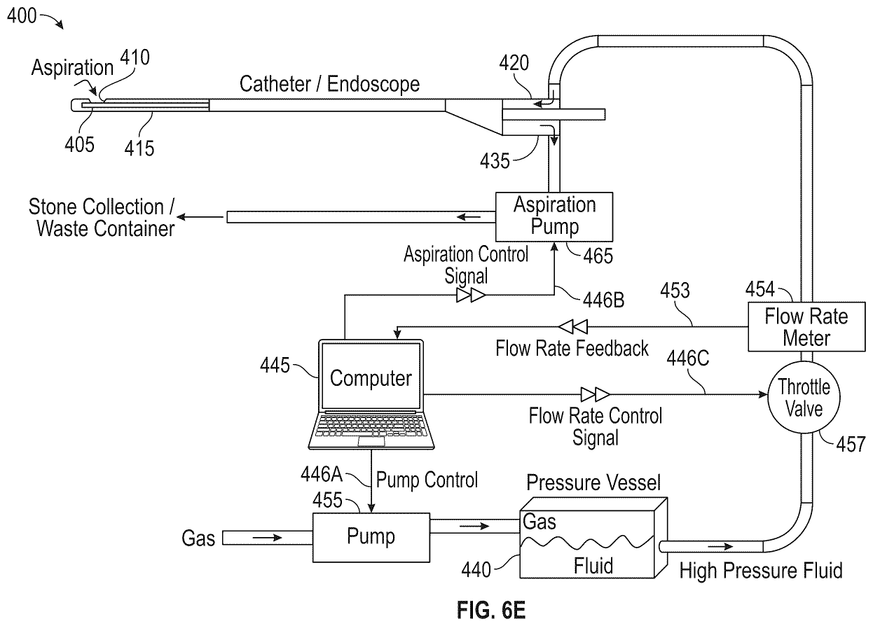

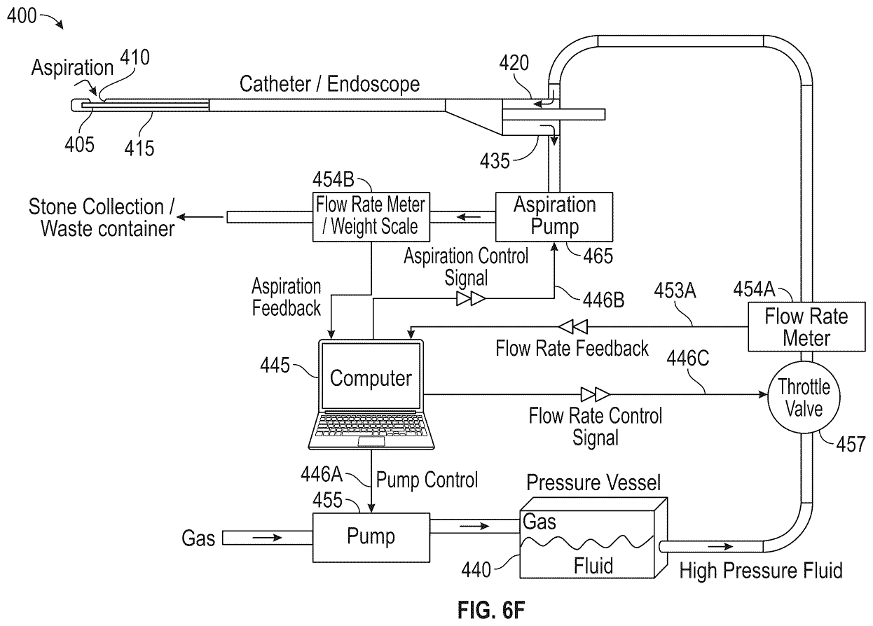

In some cases, the instrument 200 can be an aspiration catheter disposed within an outer sheath 260 (which may in some cases be a lumen of a multilumen trocar or access catheter, such as shown in FIGS. 6A and 6B. In some cases, the instrument 200 may be axially and/or rotationally movable within the outer sheath 260.

In some cases, the outer sheath 260 may be axially and/or rotationally movable with respect to the evacuation tube 205. Such configurations, for example, may enable adjustment of an angular orientation of a distal end 270 of the instrument 200 and exposure of at least one of the one or more aspiration port openings 210 and, optionally, one or more vent port openings 215 to the environment external to the outer sheath 260 when the instrument 100 is in operation. In some cases, the axial movement of the instrument 100, with respect to the sheath 260, may enable an operator to control exposure of the one or more aspiration port openings 210 and/or the one or more vent port openings 215 to the surrounding environment to facilitate different modes of operation. For example, in some cases, an operator, such as a physician, may move the outer sheath 260 relative to the instrument 200 such that at least a portion of, or all of, the one or more vent port openings 215 are closed (e.g., such that no liquid may pass through the one or more vent port openings 215). In some cases, an operator, such as a physician, may move the outer sheath 260 relative to the evacuation tube 205 such that at least a portion of, or all of, the one or more vent port openings 215 are open (e.g., such that liquid may pass through the one or more vent port openings 215).

FIGS. 4B-4E illustrate example ingress and egress flow patterns of an aspiration catheter in response to a position of the outer sheath 260. For example, as illustrated in FIGS. 4B-4E, the one or more vent port openings 215 and/or the aspiration port openings 210 may be selectively opened or closed during operation of the device (e.g., using an outer sheath 260).

As shown in FIG. 4B, the outer sheath 260 may not be disposed around the aspiration catheter. Accordingly, the one or more vent port openings 215 and the aspiration port openings 210 may be unobstructed. The unobstructed one or more vent port openings 215 and unobstructed one or more aspiration port openings 210 may provide for a fluid flow to pass through the one or more vent port openings 215 and the aspiration port openings 210. As shown in FIG. 4B, the Venturi effect may create a low-pressure zone within the evacuation lumen 235 at the aspiration port openings 210. Accordingly, external liquids (and solid deposits) may ingress into the evacuation lumen 235 via the aspiration port openings 210. In some cases, the liquid from the evacuation lumen 235 can egress from the evacuation lumen 235 to the external environment via the one or more vent port openings 215. As shown in FIG. 4B, the egress of liquid can flow in any direction away from the one or more vent port openings 215. In some cases, pressure can build up within the evacuation lumen 235 downstream from the aspiration port openings 210. Accordingly, the one or more vent port openings 215 can provide an outlet to release pressure downstream of the aspiration port openings 210. In some cases, the vent port openings 215 can allow for increased volumetric flow at the aspiration port openings 215 by providing recirculation. For example, the vent port openings 215 can increase volumetric flow at an aspiration port opening 215 positioned at the distal tip of the evacuation tube 205 as described herein. Without vent port openings 215, a vacuum source such as a peristaltic pump, as described herein, may slow the volumetric flow of fluid pulled by the instrument 200 and/or from an anatomical structure thereby reducing vacuum creation at the distal end of the evacuation tube 205. The vent port openings 215 can also be used to filter fragments and/or debris. In some cases, the vent port openings 215 can prevent the evacuation tube 205 from becoming clogged or obstructing a volumetric fluid flow through the evacuation tube 205. Accordingly, the vent port openings 215 may mitigate the risk of over-pressurization within the anatomical structure.

As shown in FIG. 4C, the outer sheath 260 can be positioned at a first axial position relative to the distal end of the aspiration catheter. In the first axial position, the outer sheath 260 can cover and/or obstruct at least a portion of the one or more vent port openings 215 while the aspiration port openings 210 remain unobstructed. In such cases, a side suction of a liquid can be generated. As shown in FIG. 4C, the Venturi effect may create a low-pressure zone within the evacuation lumen 235 at the aspiration port openings 210. Accordingly, external liquids (and solid deposits) may ingress into the evacuation lumen 235 via the aspiration port openings 210. As further shown in FIG. 4C, the liquid from the evacuation lumen 235 can egress from the evacuation lumen 235 to the external environment via the one or more vent port openings 215. However, due to the outer sheath 260 covering and/or obstructing the one or more vent port openings 215, the liquid flow can flow along the outer sheath 260. In some cases, the liquid can flow laterally toward the distal end of the aspiration catheter and the aspiration port openings 210. At least part of the egressed liquid can remain in the external volume.

As shown in FIG. 4D, the outer sheath 260 can be positioned in a second axial position relative to the distal end of the aspiration catheter. In the second axial position, the outer sheath 260 can cover and/or obstruct at least a portion of the one or more vent port openings 215 while the aspiration port openings 210 remain unobstructed. In such cases, a forced irrigation and/or repulsion of a liquid can be generated. As shown in FIG. 4C, the Venturi effect may create a low-pressure zone within the evacuation lumen 235 at the aspiration port openings 210. Accordingly, external liquids (and solid deposits) may ingress into the evacuation lumen 235 via the aspiration port openings 210. As further shown in FIG. 4D, the liquid from the evacuation lumen 235 can egress from the evacuation lumen 235 to the external environment via the one or more vent port openings 215. However, due to the outer sheath 260 covering and/or obstructing the one or more vent port openings 215, the fluid flow can flow along the outer sheath 260. In some cases, the fluid can flow laterally toward the distal end of the aspiration catheter and the aspiration port openings 210. At least part of the egressed liquid can remain in the external volume.

As shown in FIG. 4E, the outer sheath 260 can be positioned in a third axial position relative to the distal end of the aspiration catheter. In the third axial position, the outer sheath 260 can cover and/or obstruct at least a portion of the one or more vent port openings such that a substantially front-oriented (e.g., distal) suction is generated. Sheaths and other mechanisms for controlling flow through the one or more vent port openings 215 and/or aspiration port openings 210 are described in more detail herein.

In some cases, one or more aspiration port openings 110 are positioned downstream relative to a nozzle 225, as illustrated in FIG. 4A. In some cases, the one or more aspiration port openings are configured and positioned at a distal end of evacuation lumen 235. In some cases, during operation of the instrument 100, a liquid exiting the nozzle 225 can flow past at least a portion of the one or more aspiration port openings 110, thereby generating a vacuum at the one or more aspiration port openings, relative to an environment external to evacuation lumen 135. In some cases, each of the one or more aspiration port openings 110 may be characterized by a maximum opening dimension that is less than the minimum cross-sectional dimension of evacuation lumen 135 to ensure that no solid particle is aspirated that is likely to clog the evacuation lumen 135. In specific such cases, each of the one or more aspiration port openings 110 may be characterized by a maximum opening dimension that does not exceed 80% of the minimum cross-sectional dimension of evacuation lumen 135. In some cases, the maximum opening dimension does not exceed about 5%, about 10%, about 15%, about 20%, about 25%, about 30%, about 35%, about 40%, about 45%, about 50%, about 55%, about 60%, about 65%, about 70%, about 75%, or about 80% of the minimum cross-sectional dimension of evacuation lumen 135.

In some cases, the maximum opening dimension is greater than or equal to 5%, greater than or equal to 10%, greater than or equal to 20%, greater than or equal to 30%, greater than or equal to 40%, greater than or equal to 50%, greater than or equal to 60%, or greater than or equal to 70% of the minimum cross-sectional dimension of evacuation lumen 135. In some cases, the maximum opening dimension is less than or equal to 80%, less than or equal to 70%, less than or equal to 60%, less than or equal to 50%, less than or equal to 40%, less than or equal to 30%, less than or equal to 20%, less than or equal to 10%, or less than or equal to 5% of the minimum cross-sectional dimension of evacuation lumen 135.

With reference to FIG. 4A, the instrument 200 includes a nozzle 225 with an outlet 230 disposed within or immediately or in close proximity upstream of an inlet of the lumen of the evacuation lumen 235. In some cases, the outlet 230 may be configured and positioned at or near a distal end 270 of the instrument 200 such that the outlet 230 of the nozzle 225 faces and ejects a liquid jet directed towards a proximal end 280 of the instrument 200. In some cases, one or more aspiration port openings 210 may be positioned closer to the proximal end 280 of the instrument 200 than the outlet 230. The one or more vent port openings 215 can be generally positioned closer to the proximal end 280 of the instrument 100 than the one or more aspiration port openings 210.

In some cases, a portion of the liquid aspirated by the instrument 100 through the one or more aspiration port openings 210 can be ejected from the evacuation lumen 235 of the evacuation tube into a surrounding environment via the one or more vent port openings 215 while the instrument is in operation. In some cases, greater than or equal to 1%, greater than or equal to 5%, greater than or equal to 10%, greater than or equal to 15%, greater than or equal to 20%, greater than or equal to 30%, greater than or equal to 40%, or greater than or equal to 50% of the liquid aspirated by the instrument is ejected from the lumen of the evacuation lumen 235 into a surrounding environment via each of the one or more vent port openings 215 while the instrument 200 is in operation. In some cases, less than or equal to 50%, less than or equal to 40%, less than or equal to 30%, less than or equal to 20%, less than or equal to 15%, less than or equal to 10%, or less than or equal to 5%, or less than or equal to 1%, of the liquid aspirated by the instrument 200 is ejected from the evacuation lumen 235 into a surrounding environment via each of the one or more vent port openings 215 while the instrument 200 is in operation. Combinations of the above-referenced ranges are also possible (e.g., greater than or equal to 1% and less than or equal to 50%). For example, referring again to FIGS. 4B-4E, operational modes which permit liquid ejected into the surrounding environment which is aspirated by the instrument 200 may prevent e.g., excess pressure and/or volume from accumulating within the surrounding organ during operation of the instrument.

In some cases, the instrument is part of an operation and control system (see, for instance, FIGS. 6A-6F) that further includes a controller (e.g., controller 245 of FIG. 4A) configured to operate a liquid source (e.g., liquid source 240 of FIG. 4A) to create pressure and flow conditions for the delivered liquid so that—in combination with adjustment of the relative axial position of the instrument 200 within a surrounding sheath 260 or channel as depicted in FIG. 4B for instruments so configured—the one or more vent port openings 215 ejects at least a portion of the liquid volume supplied by the liquid source 240 from the evacuation lumen 235 of the evacuation tube 205 into the location internal to the subject (e.g., patient). By controlling ingress and egress of liquid in this manner, favorable recirculation flows within the organ (e.g., kidney) for visualization, capture, and retaining solid deposits can be established, and, advantageously, total pressure and liquid volume conditions within the organ or operating space can be balanced and maintained within safe, physiological operational limits. More detailed operational and control systems for this purpose are illustrated in FIGS. 6A-6F and described in more detail herein. For purposes of this disclosure, the terms “capturing” or “retaining” do not require that the biological object and/or solid deposits be physically and fixedly secured to an aspiration port opening. Instead, “capturing” or “retaining” can mean continually attracting the biological objects and/or solid deposits toward the aspiration port opening.

In some cases, during at least some periods during operation, a volume of liquid ejected back into the location internal to the subject (e.g., via the one or more vent port openings 215) can be less than or equal to a volume of the liquid aspirated into evacuation lumen 235 when the system is in operation (e.g., via the one or more aspiration port openings 210). In some cases, a volume of liquid ejected back into the location internal to the subject is greater than or equal to the volume of the liquid aspirated into the evacuation lumen 235 when the system is in operation. In an example set of cases, averaged over the total time of operation of the system, the volume of liquid ejected is substantially the same as the volume of liquid aspirated (e.g., the volume ejected is within less than or equal to 10%, less than or equal to 5%, less than or equal to 2%, or less than or equal to 1% of the volume aspirated).

In some cases, a total flow rate of an aspirated liquid through the one or more aspiration port openings 210 during at least some periods during operation is greater than or equal to a total flow rate of the liquid egress into the operating space through the one or more vent port openings 215. In some cases, during at least some periods during operation a total flow rate of a liquid through the one or more aspiration port openings 210 is greater than or equal to a total flow rate of the liquid through the one or more vent port openings 215. In an example set of cases, integrated over substantial operating times, the total flow rate of liquid ejected is substantially the same as the total flow rate of liquid aspirated (e.g., the total flow rate of liquid ejected via the one or more vent port openings 215 is within less than or equal to 10%, less than or equal to 5%, less than or equal to 2%, or less than or equal to 1% of the total flow rate of the liquid aspirated via the one or more aspiration port openings 210).

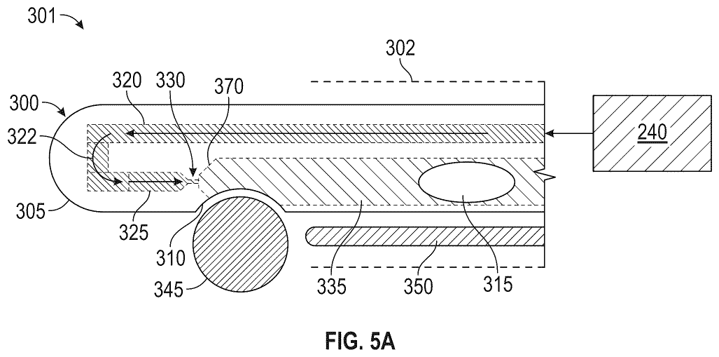

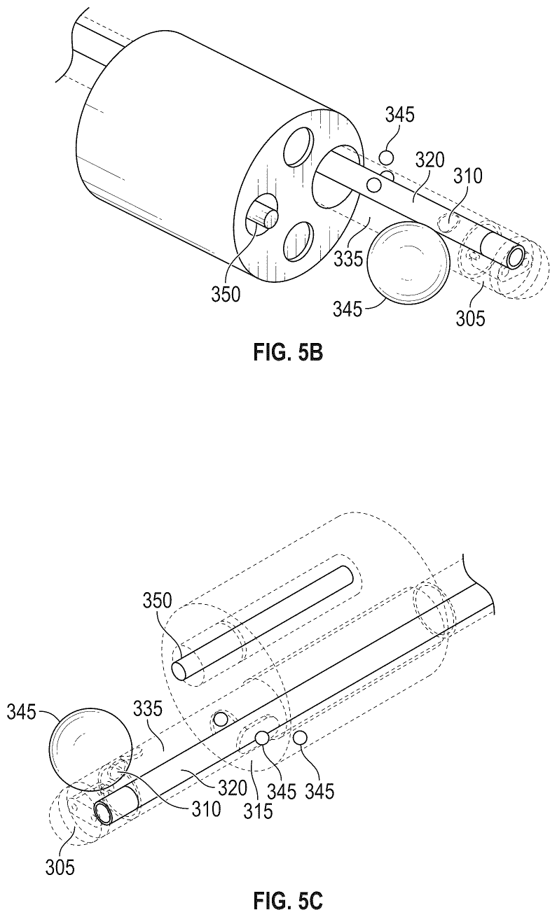

In some cases, the controller 245 is configured to operate the liquid source 240 such that a pressure and/or volume of the surrounding liquid during operation of the instrument 200 is regulated, as alluded to herein. For example, in some cases, the controller 245 may be configured to operate the liquid source 240 and other operational parameters such that a pressure and/or volume of the surround liquid during operation of the instrument 200 is within less than or equal to 50% of an initial pressure and/or initial volume of the surrounding liquid. Without wishing to be bound by any particular theory, control over the pressure and/or volume of the liquid source may be useful to ensure patency of the location within the subject, such as the pelvis or calyces of the kidney and/or the ureter (e.g., to ensure the location within the subject does not substantially collapse or expand while the instrument is in operation). In some cases, the controller 245 can be configured to operate the liquid source 240 such that the pressure and/or volume of the surrounding liquid during operation of the instrument 200 is within to 50%, within 40%, within 30%, within 20%, within 10%, or within 5% of an initial pressure and/or initial volume of the surrounding liquid at any point during the operation of the instrument 200. Referring again to the example operational modes in FIG. 4B, e.g., in cases in which the instrument 200 is axially and/or rotationally movable within a surrounding outer sheath 260 or with a guide catheter (e.g., 302 in FIG. 5A), the ingress and/or egress of a liquid through the one or more aspiration port openings 210 and/or the one or more vent port openings 215 may be controlled (e.g., to control volume and/or pressure balance and/or patterns of recirculation of the liquid). In some such cases, control of the ingress and/or egress of liquid through the one or more aspiration port openings 210 and/or the one or more vent port openings 215 may advantageously provide desirable control of liquid pressure, flow, and/or volume balance and/or control of the liquid supply.

As described herein, in some cases, the devices and systems are suitable for use in a surgical procedure e.g., at a location internal to a subject. In some cases, the devices and systems described herein are suitable for use at a location internal to a subject at least partially filled with a surrounding liquid, such that the aspiration port openings 210 and, optionally, the one or more vent port openings 215 are submerged during operation. In some cases, the devices and/or systems described combine any one or more of the Venturi-assisted medical instruments or liquid jet powered aspiration medical instruments disclosed herein and an ablation tool (e.g., a laser, an ultrasound ablation tool (such as, high-intensity focused ultrasound (HIFU) tool) or the like). In some cases, the Venturi-assisted medical instrument or a liquid jet powered aspiration medical instrument disclosed herein, and an ablation tool are on the same instrument.

FIG. 5A illustrates a system 301 including an ablation system. For example, as illustrated in FIG. 5A, the system 301 can include an aspiration instrument 300, a sheath 302, and an ablation instrument 350. In some cases, the aspiration instrument 300 can be provided via a catheter and can be liquid jet powered. The aspiration instrument 300 can include a nozzle 325 configured and positioned to direct a liquid jet into an evacuation lumen 335 to create a vacuum at an aspiration port opening 310, which is in fluidic communication with the evacuation lumen 335. In some cases, the evacuation lumen 335 can be positioned within and/or in fluidic communication with the sheath 302. The sheath 302 can be a guide catheter or trocar. In some cases, the sheath 302 can in fluid communication with one or more fluid sources. For example, the sheath 302 may be in fluid communication with a liquid source 340. The liquid source 340 can also be referred to as a liquid supply or a liquid reservoir. In some cases, a liquid reservoir can be a body or housing defining a volume to contain a liquid body. Additionally or alternatively, the liquid source 340 can be referred to as a liquid supply, a fluid source, a fluid supply, and/or a fluid reservoir. In some cases, the sheath 302 can be in fluid communication with a vacuum source. For example, the sheath 302 may be configured to apply an external vacuum (e.g., a suction force beyond that created by the aspiration resulting from the liquid jet) to the evacuation lumen 335. In other cases of this or any other Venturi-assisted medical instrument or a liquid jet powered aspiration medical instrument disclosed herein, an evacuation lumen 335 may be connected with its proximal end in fluidic communication with an external vacuum to supplemental or assist or replace aspiration created by liquid jet aspiration. In some cases, the one or more aspiration port openings 310 can be selectively sized and shaped to retain and immobilize a solid deposit 345 of predetermined size and shape within the enclosed location of the subject (e.g., a kidney stone of measured size).

In some cases, an ablation instrument 350 can form part of, or is functionally and structurally integrated with, aspiration instrument 300. In some cases, the ablation instrument 350 can include an ablation device that is movable with respect to the aspiration instrument 300. Such configurations may be useful, for example, to enable an operator of the system to position the ablation device in proximity with the one or more aspiration port openings 310 (holding a solid deposit 345, for example) to pulverize the solid deposit 345. In some cases, the ablation instrument 350 can be operably linked to the aspiration instrument 300 (e.g., the aspiration instrument 300 and the ablation instrument 350 can be mechanically coupled to a common component, e.g., the sheath 302 as illustrated and/or a guide catheter/trocar). In some cases, aspiration instrument 300 and ablation instrument 350 can be separate instruments.

In some cases, the evacuation lumen 335 can include one or more aspiration port openings 310 and/or one or more vent port openings 315. In some cases, the one or more aspiration port openings 310 can be positioned at or near a distal end 370 of evacuation lumen 335. The one or more vent port openings 315 can be configured and positioned proximal to the aspiration port openings 310 on the evacuation tube 305.

Other configurations are also possible. In some cases, the system can be a multi-lumen system. FIGS. 5B and 5C show example configurations of a multi-lumen system, as descried herein. For example, in some cases, the system can include a liquid supply lumen 320 disposed within an evacuation tube 305. Additionally, or alternatively, in some cases, the system can include a dual lumen sheath. In some cases, the dual lumen sheath can include a first lumen including a movable liquid jet powered aspiration instrument and a second lumen including a movable ablation instrument 350. The aspiration instrument may be any suitable aspiration instrument known to the skilled artisan, including, but not limited to the aspiration devices of the present disclosure. For example, the aspiration instrument can be an evacuation tube 305. In some cases, the instrument can include a dual lumen evacuation tube 305 with a first lumen including evacuation lumen 335 of FIG. 5A and a second lumen including an ablation instrument (e.g., laser, ultrasonic instrument, electrohydraulic instrument, pneumatic instrument, cryo, RF, waterjet, morcellation blades, etc., such as ablation instrument 350 of FIG. 5A). In some cases, the ablation instrument can include a laser.

FIG. 6A shows a case of a system 400 including a liquid jet powered aspiration instrument 401 including a liquid supply lumen 420 configured to receive and transport a pressurized liquid to a nozzle positioned at an end of the liquid supply lumen 420 when connected to a liquid source 440. In some cases, the aspiration instrument 401 can include an evacuation tube 405 including an evacuation lumen 435. The liquid supply lumen 420 can be configured to output a liquid jet into the evacuation lumen 435. As described herein, the pressure can be inversely proportional to the velocity of the fluid. The evacuation lumen 435 can contain a high flow rate. For example, as described above the liquid jet can have a high flow rate between about 20 mL/min and 60 mL/min, which can cause formation of low pressure. In some cases, the evacuation tube 405 can further include one or more aspiration port openings 410 in the form of one or more openings in the sidewall of the evacuation tube 405. In some cases, the evacuation tube 405 can be configured so that liquid ejected from a nozzle is directed past one or more aspiration port openings 410 to generate a Venturi-created or Venturi-assisted vacuum. For example, the pressure difference between the environment and the low pressure resulting from the flow of the liquid jet can result in a relative negative pressure or suction through the one or more aspiration port openings 410. The Venturi-created or Venturi-assisted vacuum can be a first vacuum level. Accordingly, the liquid jet can create a negative pressure at the distal end of the evacuation lumen 435. In some cases, the first vacuum level is sufficient for capturing a biological object, such as a solid deposit, within the one or more aspiration port openings 410 at the location internal to the subject when the aspiration instrument 401 is in operation. In some cases, the aspiration instrument 401 can further include one or more vent port openings 415 in the side wall of the evacuation tube 405. The one or more vent port openings 415 can be positioned downstream of the one or more aspiration port opening 410 and can be configured to eject at least a portion of liquid flowing along evacuation lumen 435.

In some cases, the system 400 can further include a high-pressure fluidic pump 455 configured to pump a liquid from the liquid source 440 into the liquid supply lumen 420. In some cases, a vacuum source 465 can be connected at a proximal end of evacuation lumen 435. The vacuum source 465 can be configured to assist aspiration from the evacuation lumen 435 by pumping fluid from the evacuation lumen 435 into a waste container. The vacuum source 465 can generate a second vacuum level. For example, the vacuum source 465 can create negative pressure at the proximal end of the evacuation lumen 435. In some cases, the second vacuum level can be less than the second vacuum level. For example, the second vacuum level generated by the vacuum source 465 may be result in a fluid flow rate slower than the flow rate of the liquid jet. For example, the second vacuum level may cause a fluid flow rate between about 0 mL/min and 150 mL/min. In some cases, the second vacuum level may cause a nominal fluid flow rate of about 70 mL/min. Accordingly, the vacuum source 465 can act as a flow regulator to regulate fluid flow through the evacuation lumen 435 and to counteract the higher vacuum level created by the flow of the liquid jet. In some examples, another flow regulator for regulating fluid flow through the evacuation lumen 435 can be used in addition to or instead of the vacuum source 465. For example, a valve can be utilized.

A controller 445 can be programmed and configured to operate the high-pressure fluidic pump 455 and/or the vacuum source 465. In some cases, the controller 445 can be used to ensure the pressure and/or volume of the location internal to the subject remains substantially constant during operation of the system 400. In some cases, the system 400 can further include a peristaltic pump connected at a proximal end of the evacuation lumen 435. A peristaltic pump can include a pinch-and-roll mechanism to create negative pressure or suction at an inlet. In some cases, the controller 445 can be programmed and configured to operate the high-pressure fluidic pump 455 and the optional peristaltic pump, when present, such that the pressure and/or volume of the location internal to the subject remains substantially constant during operation of the system 400. In some cases, the vacuum source 465 and the peristaltic pump may be operated simultaneously in parallel or in series. In some examples, the controller 445 can be configured to control a flow regulator (such as, a valve) for regulating fluid flow through the evacuation lumen 435. In some cases, the controller 445 can be configured to control the high-pressure fluidic pump 455 and the vacuum source 465 independently.

Internal pressure within the aspiration instrument 401 can be regulated and/or controlled by the fluidic pump 455 and the vacuum source 465. In some cases, the fluidic pump 455 and the vacuum source 465 can be matched 1:1. For example, the controller 445 can be configured to operate the fluidic pump 455 and the vacuum source 465 simultaneously to regulate and control pressure within the aspiration instrument 401.

FIGS. 6B-6F show example systems with various open loop and closed loop control systems. Although some examples describe one or more sensors positioned proximally of the aspiration instrument 401, the one or more sensors may also be disposed within the aspiration instrument 401, positioned at a distal end of the aspiration instrument 401, between the distal end and the proximal end of the aspiration instrument 401. In some cases, one or more sensors can be positioned within an evacuation tube 405. In some cases, one or more sensors can be positioned within an evacuation lumen 435. The one or more sensors can be configured to monitor pressure and/or temperatures within the system 400.

FIG. 6B shows an example implementation of the system 400 including at least an open loop control of one or more pumps. In some cases, the one or more pumps can include a displacement pump and/or an aspiration pump. For example, the system 400 can include a high-pressure fluidic pump 455 and a vacuum source 465. The controller 445 can be in electrical communication with the one or more pumps. For example, as shown in FIG. 6B, the controller 445 can be configured to transmit a first control signal 446A to the high-pressure fluidic pump 455 and a second control signal 446B to the vacuum source 465. In such cases, the first control signal 446A can control the fluid flow of the liquid from the liquid source 440 toward to evacuation tube 405 and the second control signal 446B can control the aspiration assistance from the vacuum source 465. In some examples, a flow regulator can be used in addition to or in place of the vacuum source 465, as described herein. As further shown in FIG. 6B, the controller 445 can be configured to operate without receiving a feedback signal. Open loop control can operate without requiring feedback from sensors. Accordingly, the system can implement a simple control system without sensors thereby reducing costs. The open loop control can also passively control fluid flow through the system without actively changing flow rates based on sensed or measured values.

FIG. 6C shows an example implementation of the system 400 including at least an open loop control of a first set of one or more pumps and a closed loop control of a second set of one or more pumps. In some cases, the first set of one or more pumps can include a displacement pump. For example, the first set of one or more pumps can include a high-pressure fluidic pump 455. In some cases, the second set of one or more pumps can include an aspiration pump. For example, the second set of one or more pumps can include a vacuum source 465. The controller 445 can be in electrical communication with the first set of one or more pumps and the second set of one or more pumps. For example, as shown in FIG. 6C, the controller 445 can be configured to transmit a first control signal 446A to the high-pressure fluidic pump 455 and a second control signal 446B to the vacuum source 465. In some examples, a flow regulator can be used in addition to or in place of the vacuum source 465, as described herein. In some cases, the system 400 can further include one or more sensors. For example, the system 400 can include a pressure sensor 452. The pressure sensor can be configured to monitor the pressure at the aspiration port openings 410 and/or to detect occlusions within the evacuation lumen 435. The controller 445 can be in electrical communication with the one or more sensors. In some cases, the one or more sensors can be configured to provide a feedback signal 453 to the controller 445. For example, the pressure sensor 452 can provide the controller 445 with a pressure signal in the evacuation lumen 435. The controller 445 can adjust one or more control signals based on the feedback. Accordingly, the system 400 can provide an open loop control of the high-pressure fluidic pump 455 with inline pressure monitoring for a closed loop control of the vacuum source 465 (and/or a flow regulator). As discussed herein, open loop control can operate without requiring feedback from sensors thereby reducing costs.

FIG. 6D shows an example implementation of system 400 including at least a closed loop control of one or more pumps. In some cases, the one or more pumps can include a displacement pump and an aspiration pump. For example, the one or more pumps can include a high-pressure fluidic pump 455 and a vacuum source 465. In some examples, a flow regulator can be used in addition to or in place of the vacuum source 465, as described herein. The controller 445 can be in electrical communication with the one or more pumps. For example, the controller 445 can be configured to transmit a first control signal 446A to the high-pressure fluidic pump 455 and a second control signal 446B to the vacuum source 465 (and/or a flow regulator). In some cases, the system 400 can further include one or more sensors. For example, as shown in FIG. 6D, the system 400 can include a first flow rate meter 454A and a second flow rate meter 454B. The first flow rate meter 454A can be configured to measure a flow rate within the liquid supply lumen 420. The second flow rate meter 454B can be configured to measure a flow rate within the evacuation lumen 435. The controller 445 can be in electrical communication with the one or more sensors. In some cases, the one or more sensors can be configured to provide a feedback signal to the controller 445. For example, the first flow rate meter 454A can be configured to provide a first feedback signal 453A to the controller 445 and the second flow rate meter 454B can be configured to provide a second feedback signal 453B to the controller 445. The controller 445 can adjust one or more control signals based on the feedback. Accordingly, the system 400 can provide a closed loop control of the high-pressure fluidic pump 455 and the vacuum source 465 (and/or a flow regulator) via flow rate feedback. Closed loop control can increase accuracy of fluid flow by actively reacting to sensed or measured values. Additionally, the system can react to unexpected events detected by sensors.

FIG. 6E shows an example implementation of system 400 including at least an open loop control of one or more pumps. In some cases, the one or more pumps can include a displacement pump and an aspiration pump. For example, the one or more pumps can include a high-pressure fluidic pump 455 and a vacuum source 465. In some examples, a flow regulator can be used in addition to or in place of the vacuum source 465, as described herein. The controller 445 can be in electrical communication with the one or more pumps. For example, the controller 445 can be configured to transmit a first control signal 446A to the high-pressure fluidic pump 455 and a second control signal 446B to the vacuum source 465 (and/or a flow regulator). In some cases, the system 400 can further include one or more valves. For example, the system 400 may include a throttle valve 457. The controller 445 can be in electrical communication with the one or more valves. For example, the controller 445 may be configured to transmit a third control signal 446C to the throttle valve 447. In some cases, the system 400 can further include one or more sensors. For example, as shown in FIG. 6E, the system 400 can include a flow rate meter 454. The flow rate meter 454 can be configured to measure a flow rate within the liquid supply lumen 420. The controller 445 can be in electrical communication with the one or more sensors. In some cases, the one or more sensors can be configured to provide a feedback signal to the controller 445. For example, the flow rate meter 454 can be configured to provide a feedback signal 453 to the controller 445. The controller 445 can adjust one or more control signals based on the feedback. Accordingly, the system 400 can provide an open loop control of the vacuum source 465 with inline fluid flow monitoring for a closed loop control of the high-pressure fluidic pump 455 and/or the throttle valve 457. Hybrid loop control implement both open loop and closed loop control. Select parts of the system may not include sensors or feedback from sensors to actively control various actuators while other select components of the system may receive feedback from sensors to actively control other various actuators. A hybrid loop control can include the advantages of both open loop and closed loop by providing accurate control to sensitive components while reducing costs.

FIG. 6F shows an example implementation of system 400 including at least closed loop feedback control of one or more pumps and a pressure vessel. In some cases, the one or more pumps can include a displacement pump and an aspiration pump. For example, the one or more pumps can include a high-pressure fluidic pump 455 and a vacuum source 465. In some examples, a flow regulator can be used in addition to or in place of the vacuum source 465, as described herein. The controller 445 can be in electrical communication with the one or more pumps. For example, the controller 445 can be configured to transmit a first control signal 446A to the high-pressure fluidic pump 455 and a second control signal 446B to the vacuum source 465 (and/or a flow regulator). In some cases, the system 400 can further include one or more valves. For example, the system 400 may include a throttle valve 457. The controller 445 can be in electrical communication with the one or more valves. For example, the controller 445 may be configured to transmit a third control signal 446C to the throttle valve 447. In some cases, the system 400 can further include one or more sensors. For example, as shown in FIG. 6F, the system 400 can include a first flow rate meter 454A and a second flow rate meter 454B. The first flow rate meter 454A can be configured to measure a flow rate within the liquid supply lumen 420. The second flow rate meter 454B can be configured to measure a flow rate within the evacuation lumen 435. The controller 445 can be in electrical communication with the one or more sensors. In some cases, the one or more sensors can be configured to provide a feedback signal to the controller 445. For example, the first flow rate meter 454A can be configured to provide a first feedback signal 453A to the controller 445 and the second flow rate meter 454B can be configured to provide a second feedback signal 453B to the controller 445. The controller 445 can adjust one or more control signals based on the feedback. Accordingly, the system 400 can provide a closed loop control of the high-pressure fluidic pump 455 and the vacuum source 465 (and/or a flow regulator) via flow rate feedback and can provide open loop control of the throttle valve 457. As discussed herein, hybrid loop control can include the advantages of both open loop and closed loop by providing accurate control to sensitive components while reducing costs.

Other combinations of these features and/or other configurations are also possible. Those of ordinary skill in the art would be capable of selecting suitable open loop control systems, closed loop control systems, flow rate feedback systems, pumps, pressure vessels, and the like based upon the teachings of this specification.

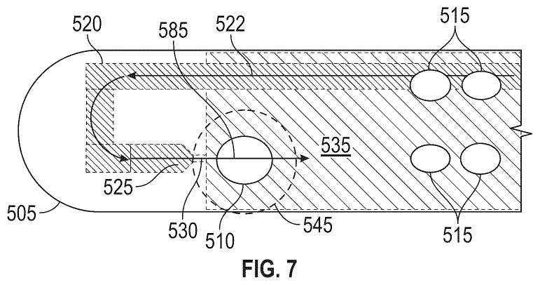

Aspects of the disclosure further relate to methods of using the devices disclosed herein. FIG. 7 illustrates a distal end of an evacuation tube 505, which can be similar to the evacuation tubes 105 or 205 or any of the evacuation tubes described herein except for the differences described herein. In some cases, the methods relate to capturing a biological object such as a solid deposit 545 (e.g., a kidney stone) at a location internal to a subject (e.g., a kidney), for example, using any of the devices disclosed herein. For example, as shown in FIG. 7 the methods can include forming a liquid jet 585 with a nozzle 525 and directing the liquid jet 585 from an outlet 530 into the evacuation lumen 535 of the evacuation tube 505 and proximate to the one or more aspiration port openings 510 in a side wall of the evacuation lumen 535 of the evacuation tube 505 at the one or more aspiration port openings 510.

Venturi-created or Venturi-assisted vacuum, as described herein, is created because passing a liquid (e.g., physiological liquids such as water or saline), through a constriction (e.g., a nozzle) increases the velocity of the physiological fluid in, accordance with the principle of mass continuity, which is balanced by a drop in the static pressure, in accordance with the conservation of mechanical energy (e.g., Bernoulli's principle). As shown illustratively in FIG. 3C, this process reduces, in some cases, the pressure inside the evacuation lumen, relative to pressure of an environment outside of the device (e.g., the pelvis or calyces of the kidney), resulting in a vacuum proximate the one or more aspiration port openings.

In some cases, the Venturi-created or Venturi-assisted vacuum produces a suction force of greater than or equal to 50 mmHg and less than or equal to 600 mmHg. In some cases, the vacuum generated produces a suction force is greater than or equal to 50 mmHg, greater than or equal to 100 mmHg, greater than or equal to 200 mmHg, greater than or equal to 300 mmHg, greater than or equal to 400 mmHg, greater than or equal to 500 mmHg, or greater than or equal to 600 mmHg. In some cases, the suction force is less than or equal to 600 mmHg, less than or equal to 500 mmHg, less than or equal to 400 mmHg, less than or equal to 300 mmHg, less than or equal to 200 mmHg, less than or equal to 100 mmHg, or less than or equal to 50 mmHg. The desired suction force can be determined by balancing various factors including the requisite force to aspirate a biological object and safety to the patient. In some cases, the suction force can be selected to balance adequately aspirating biological objects, such as urinary calculi, without injuring or causing damage to the surrounding tissue. For example, the suction force may be sufficiently strong to pull loose debris and fragments of a urinary calculus toward an aspiration port opening but not so strong as to aspirate the surrounding tissue and/or mucosal wall.

In some cases, the methods can include balancing the suction force with the internal pressure at the location internal to the subject, for example, to ensure that the location internal to the subject (such as, an organ) does not collapse on itself during operation of the device. As such, in some cases, the methods include operating the device such that the pressure within the location internal to the subject is maintained within 10% (e.g., within 5%, within 1%) of an initial pressure at the location internal to the subject, while the device is in operation.

In some cases, the methods can further include capturing and retaining the biological object such as a solid deposit 545 at the one or more aspiration port openings 510. The biological object may be any suitable biological object capable of being captured using the Venturi-created or Venturi-assisted vacuum disclosed herein. For example, in some cases, the biological object is a solid deposit 545 such as a stone known to form in the kidneys, bladder, prostate gland, gallbladder, salivary glands, and pancreas (e.g., kidney stone, bladder stone, prostatic calculi, gallstones, salivary stones, biliary stones, pancreatic stones). The Venturi-created or Venturi-assisted vacuum can be used to attract the biological object toward an aspiration port opening 510. The aspiration port opening 510 can be configured to receive the biological object through the aspiration port opening, if the biological object is smaller than the aspiration port opening. The aspiration port opening 510 can capture and retain the biological object by continually attracting the biological object toward the aspiration port opening 510 if the biological object is larger than the aspiration port opening 510. In some cases, the biological object may be fixedly secured to the aspiration port opening 510 via aspiration. Other biological objects may also be captured using the Venturi-assisted and Venturi-created vacuums disclosed herein, for example, blood clots, tissue samples (e.g., such as those obtained by biopsy), tissue debris, cellular debris (e.g., endometrial cells, prostate cells), necrotic tissue, mucous, pleghym, cysts, emboli, bone marrow, fetal cells, eggs, and portions and/or components thereof. In some cases, the biological object is the byproduct of a procedure (e.g., procedures that use ablation techniques/tools) such as cardiac procedures (e.g., ablation of heart tissue), pulmonary procedures (e.g., ablation of tumors), liver procedures (e.g., ablation of tumors), kidney procedures (e.g., ablation of tumors), endometrial ablation procedures, prostate ablation procedures, brain tumor ablation procedures, or gastrointestinal procedures (e.g., polyp removal). Other types of biological objects are also possible.

In some cases, the methods also include venting into the location internal to the subject, a selectable fraction of a total volumetric flow rate of liquid aspirated by the Venturi-created or Venturi-assisted vacuum. In some cases, the fraction of the total volumetric flow rate of liquid aspirated by the Venturi-created or venture-assisted vacuum that is vented is selected to maintain the pressure and/or volume within the location internal to the subject remains substantially constant, as described herein.

In some cases, a vented liquid exits the evacuation lumen 535 into the location surrounding the evacuation tube 505 through the one or more vent port openings 515 in a side wall of the evacuation lumen 535 of the evacuation tube 505 that is located downstream of the one or more aspiration port openings 510.

In some cases, the methods further include removing the biological object such as a solid deposit 545 at a location internal to a subject, for example, using the one or more devices disclosed herein. In some cases, the methods include forming a liquid jet 585 with the nozzle 525 and directing liquid jet 585 into the evacuation lumen 535 of the evacuation tube 505 and proximate the one or more aspiration port openings 510 in a side wall of the evacuation lumen 535 in the evacuation tube 505 to generate a Venturi-created or Venturi-assisted vacuum at the one or more aspiration port openings 510.

In some cases, the methods further include using the suction force generated by the Venturi-created or Venturi-assisted vacuum to capture and retain biological object such as a solid deposit 545 at the one or more aspiration openings 510.

In some cases, the methods further include ablating the biological object such a solid deposit 545 into a plurality of (smaller) particles using an ablating instrument. In some cases, the ablating instrument can be a laser, however, other ablating instruments are also contemplated herein. Non-limiting cases of suitable ablating instruments include ultrasonic instruments, electrohydraulic instruments, and pneumatic instruments. In some cases, the plurality of particles is configured to be removed from the location internal a subject via the Venturi-based vacuum at the one or more aspiration port openings.

The described examples are not intended to be limiting in any way, and the skilled artisan will understand, based upon the teachings of this specification, that any one of the devices (e.g., instruments) and/or systems, and/or methods disclosed herein may further include one or more additional cases, such as those described in detail herein. For example, in some cases, the devices (e.g., instruments) and/or systems described herein can include an evacuation tube (e.g., evacuation tube 105, 205, 305, 405, or 505) with an outer diameter of greater than or equal to 1 mm and less than or equal to 10 mm. In some cases, the outer diameter of the evacuation tube 505 can be greater than or equal to 0.5 mm, greater than or equal to 1 mm, greater than or equal to 2 mm, greater than or equal to 3 mm, greater than or equal to 4 mm, greater than or equal to 5 mm, greater than or equal to 6 mm, greater than or equal to 7 mm, greater than or equal to 8 mm, greater than or equal to 9 mm, or greater than or equal to 9.5 mm. In some cases the outer diameter of the evacuation tube 505 can be less than or equal to 10 mm, less than or equal to 9 mm, less than or equal to 8 mm, less than or equal to 7 mm, less than or equal to 6 mm, less than or equal to 5 mm, less than or equal to 4 mm, less than or equal to 3 mm, less than or equal to 2 mm, less than or equal to 1 mm, or less than or equal to 0.5 mm. Combinations of the above-referenced ranges are possible (e.g., greater than or equal to 1 mm and less than or equal to 10 mm). Other ranges are also possible. The diameter of the evacuation tube 505 can be determined based on a variety of factors including flow rates, desired pressures for aspiration, maneuverability, efficiency, and safety for the patient. Smaller diameters can provide higher flow velocities and correspondingly lower pressures for enhanced suction/aspiration for a given flow rate through the liquid supply lumen 520 and/or nozzle. Smaller diameters can also maintain tip deflectability for endoscopes such as ureteroscopes. Larger diameters can provide larger aspiration port openings 510 to aspirate larger biological objects, increase efficiency, and reduce the duration of a procedure. Accordingly, the outer diameter can be selected by balancing several considerations.

In some cases, the devices (e.g., instruments) and/or systems described herein can include greater than or equal to two and less than or equal to six aspiration port openings (e.g., aspiration port openings 510). In some cases, the number of aspiration port openings 510 can be greater than or equal to 2, greater than or equal to 3, greater than or equal to 4, greater than or equal to 5, or greater than or equal to 6. In some cases, the number of aspiration port openings is less than or equal to 6, less than or equal to 5, less than or equal to 4, less than or equal to 3, or less than or equal to 2. The quantity of the aspiration port openings 510 can be determined based on a variety of factors including fluid recirculation, and maneuverability of the device and/or biological object. The quantity of aspiration port openings 510 can contribute to recirculation of fluid. In some embodiments, the quantity of aspiration port openings 510 can be selected to provide adequate recirculation. The quantity of aspiration port openings 510 can also provide multiple locations for attracting biological objects including debris and fragments toward the evacuation tube 505.

In some cases, the one or more aspiration port openings (e.g., one or more aspiration port openings 510) can be placed in a particular configuration along and/or around the evacuation lumen. The one or more aspiration port openings 510 may be placed in any suitable configuration on evacuation lumen known to the skilled artisan. For example, in some cases, the one or more aspiration port openings 510 may be placed in the circular configuration around the circumference of the evacuation lumen. In such cases, the circular configuration can reduce the chance of clogging the evacuation lumen and can enable rotation of the evacuation tube 505 without affecting the suction around the evacuation tube 505. Additionally or alternatively, the aspiration port openings 510 can be in a line of sight (LOS) of an endoscopic camera and/or an ablation device as described herein. In such cases, debris and/or fragments of biological objects fragmented from the biological object can remain in full view as the ablation device fragments the biological object.

In some cases, each of the one or more aspiration port openings 510 can have an average cross-sectional dimension of greater than or equal to 0.01 mm, greater than or equal to 0.025 mm, greater than or equal to 0.05 mm, greater than or equal to 0.1 mm, greater than or equal to 0.5 mm, greater than or equal to 1 mm, greater than or equal to 1.5 mm, greater than or equal to 2 mm, greater than or equal to 4 mm, or greater than or equal to 5 mm. In some cases, each of the one or more aspiration port openings 510 can have an average cross-sectional of less than or equal to 6 mm, less than or equal to 5 mm, less than or equal to 4 mm, less than or equal to 2 mm, less than or equal to 1.5 mm, less than or equal to 1 mm, less than or equal to 0.5 mm, less than or equal to 0.1 mm, or less than or equal to 0.05 mm. Combinations of the above referenced ranges are also possible (e.g., greater than or equal to 0.01 mm and less than or equal to 6 mm, greater than or equal to 0.05 mm and less than or equal to 1 mm). Other ranges are also possible. Selecting a size for the one or more aspiration port openings 510 can be determined by balancing various factors including the size of the biological objects and available suction force. Larger aspiration port openings 510 can allow larger particles and biological materials to pass through the aspiration port openings 510. Large biological objects may become stuck and risk blocking aspiration flow. Accordingly, in some embodiments, the aspiration port openings 510 can be limited in size to prevent large biological objects from entering the evacuation lumen. Additionally or alternatively, smaller aspiration port openings 510 may help create more suction force at the distal tip due to an increased velocity of the liquid through the smaller aspiration port openings 510.

The one or more aspiration port openings 510 may be of any suitable cross-sectional shape. Non-limiting examples of suitable cross-sectional shapes include triangles, squares, rectangles (e.g., having any suitable aspect ratio) circles, ovals, polygons (e.g., pentagons, hexagons, heptagons, octagons, nonagons, dodecagons, or the like), rings, irregular shapes, or the like.

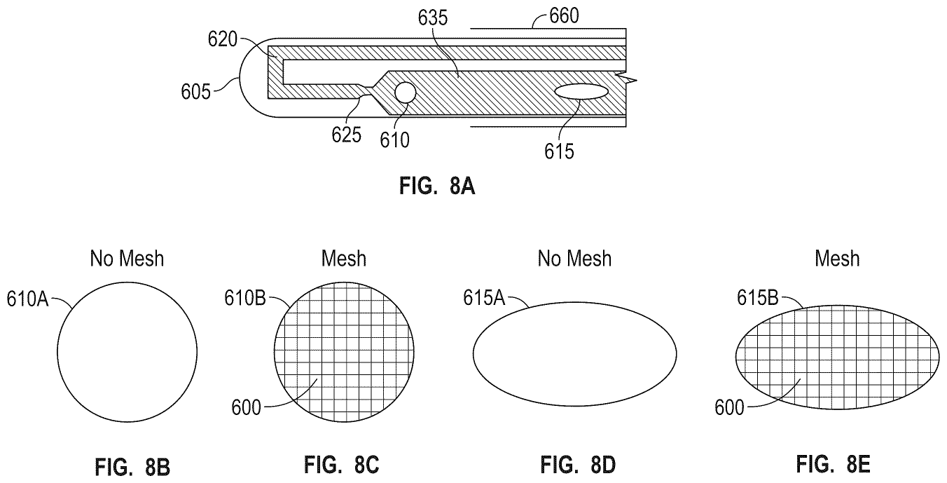

In some cases, one or more aspiration port openings 610 and/or one or more vent port openings 615 can include a mesh 600. FIG. 8A shows an evacuation tube 605 having a one or more aspiration port openings 610, one or more vent port openings 615, a liquid supply lumen 620, a nozzle 625, and an evacuation lumen 635. The evacuation tube 605 can be the same or similar to any of the evacuation tubes described herein, such as the evacuation tubes 105 or 205. In some cases, the evacuation tube 605 can be positioned within an outer sheath 660.

FIG. 8B shows one of the one or more aspiration port openings 610 without a mesh 600. Accordingly, the aspiration port opening 610A can be unobstructed. Without a mesh 600, particles can freely ingress or egress through the aspiration port opening 610A. In such cases, the only limitation on the particle sizes that can be received by the aspiration port opening 610A is the outer periphery of the aspiration port opening 610A.

FIG. 8C shows one of the one or more aspiration port opening 610 having a mesh 600. Accordingly, the aspiration port opening 610B can be at least partially obstructed. With the mesh 600, particles can be prevented from freely ingressing or egressing through the aspiration port opening 610B.

FIG. 8D shows one of the one or more vent port openings 615 without a mesh 600. Accordingly, the vent port opening 615A can be unobstructed. Without a mesh 600, particles can freely ingress or egress through the vent port opening 615A. In such cases, the only limitation on the particle sizes that can be received by the vent port opening 615A is the outer periphery of the vent port opening 615A.

FIG. 8E shows one of the one or more vent port openings 615 having a mesh 600. Accordingly, the vent port opening 615B can be at least partially obstructed. With the mesh 600, particles can be prevented from freely ingressing or egressing through the vent port opening 615B.

The mesh 600 shown in FIGS. 8C and 8E can have any suitable pore size and those of ordinary skill in the art would be capable of selecting suitable pore sizes, based upon the teachings of this specification. For example, in some cases, the mesh 600 can have a U.S. Mesh Number of greater than or equal to 35, greater than or equal to 50, greater than or equal to 75, greater than or equal to 100, greater than or equal 150, greater than or equal 200, greater than or equal to 250, greater than or equal 300, or greater than or equal 350. In some cases, the mesh 600 can have a U.S. Mesh Number of less than or equal to 400, less than or equal to 350, less than or equal to 300, less than or equal to 250, less than or equal 200, less than or equal 150, less than or equal to 100, less than or equal 75, or less than or equal to 50. Combinations of the above-referenced ranges are possible (e.g., greater than or equal to 35 and less than or equal to 400). Other ranges are also possible. As would be understood by one of ordinary skill in the art, the U.S. Mesh Number (sometimes referred to as a Tyler Mesh Size) is the number of holes per linear inch of a mesh. Each hole of the mesh may have any suitable size according to the U.S. Mesh Number standard (e.g., a diameter greater than or equal to 0.037 mm and less than or equal to 0.500 mm).

In some cases, the one or more aspiration port openings 610 can be configured to selectively retain and/or exclude a biological object such as a solid deposit (e.g., a plurality of kidney stones). Selective regulation of biological objects may be done using any suitable technique known to the skilled artisan.

In some cases, the devices (e.g., instruments) and/or systems described herein include greater than or equal to two and less than or equal to six vent port openings (e.g., the one or more vent port opening 215 as shown in FIG. 4A). In some cases, the number of the one or more vent port openings 615 can be greater than or equal to 2, greater than or equal to 3, greater than or equal to 4, greater than or equal to 5, or greater than or equal to 6. In some cases, the number of the one or more vent port openings 615 can be less than or equal to 6, less than or equal to 5, less than or equal to 4, less than or equal to 3, or less than or equal to 2.

In some cases, the one or more vent port openings 615 can be placed in a particular configuration along and/or around the evacuation lumen 635. The one or more vent port openings 615 may be placed in any suitable configuration on evacuation lumen 635 known to the skilled artisan. For example, in some cases, the one or more vent port openings 615 can be placed in a linear configuration along the length of the evacuation lumen 635. In some cases, the one or more vent port openings 615 can be placed in a circular configuration around the circumference of the evacuation lumen 635.

In some cases, each of the one or more vent port openings 615 can have an average cross-sectional dimension of greater than or equal to 0.01 mm, greater than or equal to 0.025 mm, greater than or equal to 0.05 mm, greater than or equal to 0.1 mm, greater than or equal to 0.5 mm, greater than or equal to 1 mm, greater than or equal to 1.5 mm, greater than or equal to 2 mm, greater than or equal to 4 mm, or greater than or equal to 5 mm. In some cases, each of the one or more vent port openings 615 can have an average cross-sectional of less than or equal to 6 mm, less than or equal to 5 mm, less than or equal to 4 mm, less than or equal to 2 mm, less than or equal to 1.5 mm, less than or equal to 1 mm, less than or equal to 0.5 mm, less than or equal to 0.1 mm, or less than or equal to 0.05 mm. Combinations of the above referenced ranges are also possible (e.g., greater than or equal to 0.01 mm and less than or equal to 6 mm, greater than or equal to 0.05 mm and less than or equal to 1 mm). Other ranges are also possible.

The one or more vent port openings 615 may be of any suitable cross-sectional shape. Non-limiting examples of suitable cross-sectional shapes include triangles, squares, rectangles (e.g., having any suitable aspect ratio) circles, ovals, polygons (e.g., pentagons, hexagons, heptagons, octagons, nonagons, dodecagons, or the like), rings, irregular shapes, or the like.

In some cases, one or more vent port openings 615 can include a mesh 600. The mesh 600 may have any suitable pore size and those of ordinary skill in the art would be capable of selecting suitable pore sizes, based upon the teachings of this specification.

In some cases, the devices (e.g., instruments) and/or systems described herein can include an outer sheath (e.g., outer sheath 660). In some cases, a liquid-jet forming aspiration device is disposed within the outer sheath 660. Any one of the liquid-jet forming aspiration devices (e.g., instrument 200) disclosed herein may be disposed within the outer sheath 660. In some cases, the liquid jet forming aspiration device can be a liquid jet forming aspiration catheter disposed within the outer sheath 660.

In some cases, the liquid jet forming aspiration device can be movable within the outer sheath 660. In some cases, the catheter can be capable of being moved axially and rotationally within the outer sheath 660, for example, to enable adjustment of an angular orientation of a distal end of the liquid jet forming aspiration catheter and exposure of the one or more aspiration port openings 610 to an environment external to the evacuation lumen 635 (e.g., the lumen of the pelvis or calyces of the kidney) when the instrument is in operation. For example, referring again to FIG. 4A, outer sheath 260 may be movable with respect to evacuation tube 205.

In some cases, the outer sheath 660 can be associated with an auxiliary device. For example, in some cases, the outer sheath 660 can be associated with a catheter, an endoscope, a trocar, or the like.

In some cases, the outer sheath 660 can be a dual lumen sheath. In some cases, the dual lumen sheath can be a dual lumen catheter or a dual lumen scope, such as a dual lumen endoscope.

In some cases, the outer sheath 660 can have an inner diameter of greater than or equal to 1 mm and less than or equal to 10 mm. In some cases, the inner diameter of the outer sheath 660 can be greater than or equal to 0.5 mm, greater than or equal to 1 mm, greater than or equal to 2 mm, greater than or equal to 3 mm, greater than or equal to 4 mm, greater than or equal to 5 mm, greater than or equal to 6 mm, greater than or equal to 7 mm, greater than or equal to 8 mm, greater than or equal to 9 mm, or greater than or equal to 9.5 mm. In some cases the inner diameter of the outer sheath 660 can be less than or equal to 10 mm, less than or equal to 9 mm, less than or equal to 8 mm, less than or equal to 7 mm, less than or equal to 6 mm, less than or equal to 5 mm, less than or equal to 4 mm, less than or equal to 3 mm, less than or equal to 2 mm, less than or equal to 1 mm, or less than or equal to 0.5 mm. Combinations of the above-referenced ranges are possible (e.g., greater than or equal to 1 mm and less than or equal to 10 mm). Other ranges are also possible. In some cases, the inner diameter of the outer sheath 660 can be substantially similar to the outer diameter of the evacuation tube 605. The outer sheath 660, as disclosed herein, may be of any suitable geometry. For example, in some cases, the outer sheath 660 may be shaped like a circle, triangle, square, diamond, or the like.

In some cases, a liquid-jet forming aspiration device can be configured to fit within a catheter. The catheter may be of any suitable size known to the skilled artisan. For example, in some cases the catheter size is between 3 Fr and 20 Fr. In some cases, the catheter size is between 2 Fr and 4 Fr. In some cases, the catheter size is 3.6 Fr.

In some cases, the controller can be adapted and configured to flow a liquid through the liquid supply lumen 620 at a flow rate of greater than or equal to between 10 mL/min and 200 mL/min. in some cases, the controller can be adapted and configured to flow a liquid through the liquid supply lumen 620 at a flow rate of greater than or equal to 10 mL/min, greater than or equal to 20 mL/min, greater than or equal to 30 mL/min, greater than or equal to 40 mL/min, greater than or equal to 50 mL/min, greater than or equal to 60 mL/min, greater than or equal to 70 mL/min, greater than or equal to 80 mL/min, greater than or equal to 90 mL/min, or greater than or equal to 100 mL/min. In some cases, the controller can be adapted and configured to flow of liquid through the liquid supply lumen 620 at a flow rate of less than or equal to 100 mL/min, less than or equal to 90 mL/min, less than or equal to 80 mL/min, less than or equal to 70 mL/min, less than or equal to 60 mL/min, less than or equal to 50 mL/min, less than or equal to 40 mL/min, less than or equal to 30 mL/min, less than or equal to 20 mL/min, or less than or equal to 10 mL/min. The flow rate through the liquid supply lumen 620 can be selected by balancing various factors including resulting suction force, heat transfer, fluid management, and risk of injury to the patient. Providing an insufficient flow rate can result in generating insufficient suction to aspirate one or more biological objects and/or providing insufficient heat transfer from the ablation device to the liquid for regulating and maintaining temperatures within the system. Accordingly, the system may be unable to remove biological objects and/or may result in injury to the patient. By comparison, providing an excessively high flow rate can overpower a vacuum source such that the outlet flow rate caused by the vacuum source cannot match the flow rate of the inlet flow rate through the liquid supply lumen 620. Thus, providing an excessively high flow rate risks overexpansion of the anatomical structure receiving the system which may result in injury to the patient. Furthermore, providing an excessively high flow rate can pose risks of injury to the patient because it may be difficult to detect a discrepancy between inflow and outflow and respond to the discrepancy in a timely manner.

In some cases, the devices (e.g., instruments) and/or systems described herein include a liquid supply lumen (e.g., liquid supply lumen 620 of FIG. 8A). In some cases, the liquid supply lumen 620 can include an inlet configured to receive a liquid at a pressure of greater than or equal to 500 psi and less than or equal to 15,000 psi. In some cases, the inlet can be configured to receive a liquid at a pressure of greater than or equal to 500 psi, greater than or equal to 1000 psi, greater than or equal to 2500 psi, greater than or equal to 5000 psi, greater than or equal to 7500 psi, greater than or equal to 10,000 psi, greater than or equal to 12,500 psi, or greater than or equal to 15,000 psi. In some cases, the inlet can be configured to receive a liquid at a pressure of less than or equal to 15,000 psi, less than or equal to 12,500 psi, less than or equal to 10,000 psi, less than or equal to 7500 psi, less than or equal to 5000 psi, less than or equal to 2500 psi, less than or equal to 1000 psi, or less than or equal to 500 psi. The pressure within the liquid supply lumen 620 can be determined by balancing various factors including material strength, liquid flow rates, maneuverability, visibility, requisite suction force, and safety to the patient. A liquid flow rate is measured as a volume of liquid passing a location for a given period of time. As described herein, velocity and pressure can be inversely related. Accordingly, the size of the liquid supply lumen 620 can be inversely proportional to the pressure that can be contained within the liquid supply lumen 620 to provide a given target flow rate. For example, the higher the pressure, the smaller the liquid supply lumen 620 can be to provide the same target flow rate as a larger liquid supply lumen 620. In some cases, smaller liquid supply lumens 620 may be more flexible than larger liquid supply lumens 620. Accordingly, maneuverability of the system can be increased by implementing a smaller liquid supply lumen 620. Excessively high pressures within the liquid supply lumen 620 can cause the liquid jet to cavitate inside the evacuation tube 605 resulting in introducing bubbles into the field of view and impairing visibility. Insufficient pressure can result in a low flow rate that is ineffective to produce adequate suction.

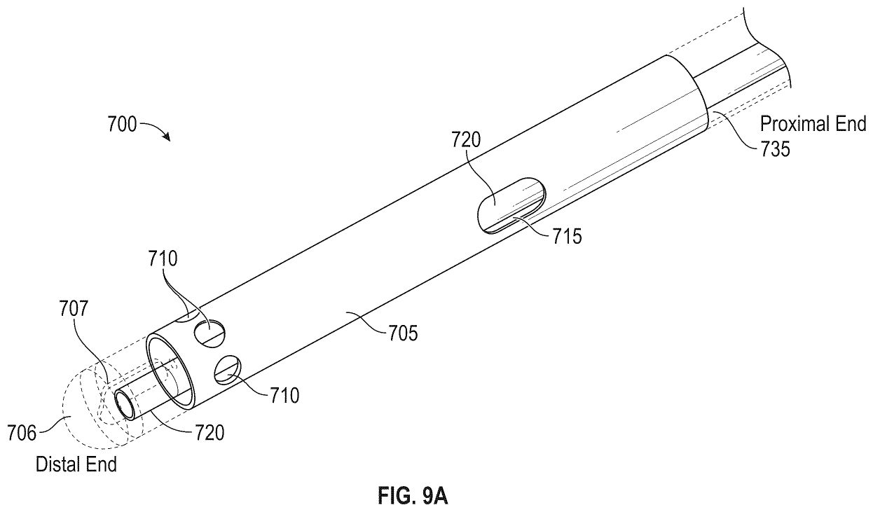

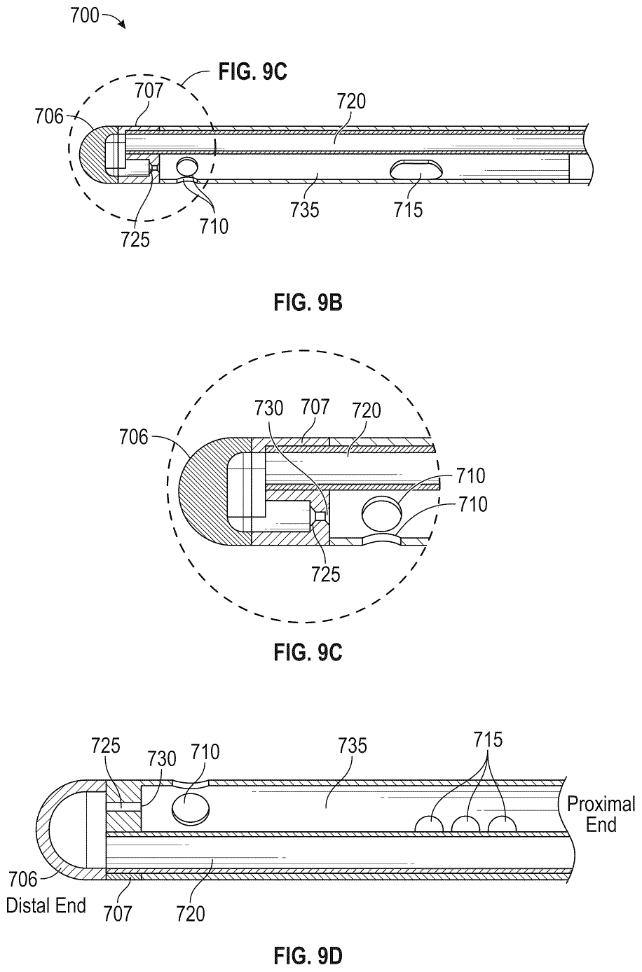

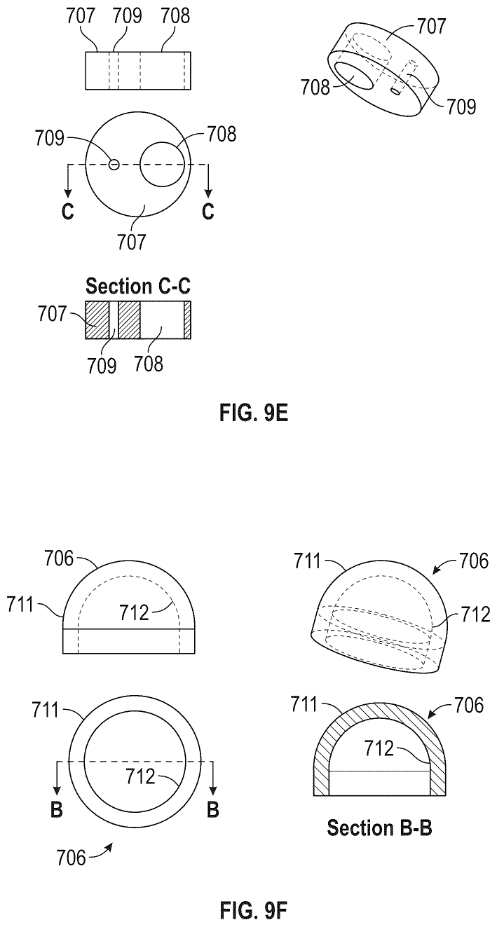

FIGS. 9A-9F, show an instrument 700 having an evacuation tube 705 coupled to an inverter cap 706. As described herein, an evacuation tube can refer to a catheter body. For example, the evacuation tube 705 can be an outer wall of a catheter. In some cases, the inverter cap 706 can be a high-pressure fluid invertor. In some cases, the inverter cap 706 can be disposed at the distal end of the instrument 700. In some cases, the inverter cap 706 can be dome-shaped. The dome shape of the inverter cap 706 may advantageously provide a rounded and smooth end that may contact the patient's internal tissue. The rounded and smooth geometry of the inverter cap 706 may prevent injury to the patient's internal tissue from axial contact. Additionally, the inverter cap 706 can have an internal geometry to assist with redirecting the liquid jet-flow from the liquid supply lumen 720 to the evacuation lumen 735. For example, the inverter cap 706 can have an internal concave structure configured to redirect a fluid flow 180 degrees.

FIG. 9A shows a perspective view of a distal end of the instrument 700. The instrument 700 can be the same or similar to other instruments described herein, such as the instrument 100 or 200. For example, the instrument 700 can include an evacuation tube 705 having a plurality of aspiration port openings 710, one or more vent port openings 715, a liquid supply lumen 720, and an evacuation lumen 735. The aspiration port openings 710, the one or more vent port openings 715, the liquid supply lumen 720, and the evacuation lumen 735 can be the same or similar to the aspiration port openings, the one or more vent port openings, the liquid supply lumens, and the evacuation lumens described herein with respect to other cases. The instrument 700 can include the inverter cap 706. In some cases, the instrument 700 can further include an inverter plate 707. In some cases, the inverter plate 707 can be disposed within the evacuation lumen proximally positioned to the inverter cap 706. For example, the inverter plate 707 can be positioned between the evacuation tube 705 and the inverter cap 706. In some cases, the inverter cap 706 can be fixedly attached to the inverter plate 707. For example, the inverter cap 706 may be laser welded to the inverter plate 707. In some cases, the inverter plate 707 can be an annular structure configured to surround the liquid supply lumen 720.

FIGS. 9B-9D show side cross-sectional views of the distal end of the instrument 700. As shown in FIG. 9B, the instrument 700 can include a nozzle 725. The nozzle 725 may include any suitable cross-sectional geometry known to the skilled artisan. Non-limiting cross-sectional geometries contemplated herein include cone geometry, bell or contoured geometry, and the annular or plug geometry.

In some cases, the nozzle 725 can include a cross-sectional dimension of greater than or equal to 50 μm or less than or equal to 200 μm. In some cases, the nozzle cross-sectional diameter can be greater than or equal to 50 μm, greater than or equal to 75 μm, greater than or equal to 100 μm, greater than or equal to 125 μm, greater than or equal to 150 μm, greater than or equal to 175 microns, or greater than or equal to 200 μm. In some cases, the nozzle cross-sectional diameter can be less than or equal to 200 μm, less than or equal to 150 μm, less than or equal to 125 μm, less than or equal to 100 μm, less than or equal to 75 μm, or less than or equal to 50 μm. In some cases, the nozzle 725 may have a cross-sectional dimension that tapers along at least a portion of the length of the nozzle 725. The size of the cross-sectional dimension of the nozzle 725 can be determined by balancing various factors including liquid flow rates, requisite suction force, and safety to the patient. As described herein, a liquid flow rate is measured as a volume of liquid passing a location for a given period of time. A larger cross-sectional dimension can provide a greater volume than a smaller cross-sectional dimension. A deficiently small cross-sectional dimension can result in inadequate liquid flow rates to create suction in a larger cross-sectional lumen such as the evacuation tube 705. By comparison, an excessively large cross-sectional dimension can require greater flow rates to generate a flow rate having adequate velocity to produce a liquid jet.

In some cases, the nozzle 725 can be configured to rotate about an axis. In some cases, the nozzle 725 is configured to rotate to any angle between +90° and −90°, relative to the axis of rotation. In some cases, rotating the nozzle 725 can change the direction of the liquid jet that is formed by passing a high-pressure fluid through the nozzle 725.

The nozzle 725 can further include an outlet 730 for directing a fluid flow from the liquid supply lumen 720 to the evacuation lumen 735. The outlet 730 may include any suitable cross-sectional shape. Non-limiting examples of suitable cross-sectional shapes include triangles, squares, rectangles (e.g., having any suitable aspect ratio) circles, ovals, polygons (e.g., pentagons, hexagons, heptagons, octagons, nonagons, dodecagons, or the like), rings, irregular shapes, or the like.

As shown in FIG. 9C, the outlet 730 can be positioned at the proximal end of a nozzle 725.

FIG. 9E shows various views of the inverter plate 707. As shown in FIG. 9E, the inverter plate 707 can have an annular structure with one or more openings extending therethrough. For example, the one or more openings can include a first opening 708 and a second opening 709. In some cases, the first opening 708 can be configured to receive and/or be part of the liquid supply lumen 720. In some cases, the second opening 709 can be configured to receive and/or be part of the nozzle 725.

FIG. 9F shows various views of the inverter cap 706. As shown in FIG. 9F, the inverter cap 706 can have a dome-shaped body. In some cases, the inverter cap 706 can have a first surface 711 and a second surface 712. The first surface 711 can be an outer surface and the second surface 712 can be an inner surface. In some cases, the second surface 712 can be configured to redirect a fluid flow. For example, fluid from the liquid supply lumen 720 may enter the volume defined by the second surface 712 and be redirected by the dome shaped second surface 712 toward the nozzle 725.

In some cases, the instrument can include one or more feedback mechanisms. In some cases, the feedback mechanism can be configured to control one or more instrument parameters in response to a change in temperature or pressure. Other parameters may also trigger the feedback mechanism, such as for example, a change in the suction force generated by the device. In some cases, the feedback mechanism can include one or more sensors to detect the proximity of the biological object, thereby aiding in accurate targeting. In some cases, the feedback mechanism adjusts the vacuum force in real-time based on the size and nature of the biological object being captured. In some cases, the feedback mechanism regulates the operating temperature of the device to maintain the optimal temperature within the location internal to the subject.

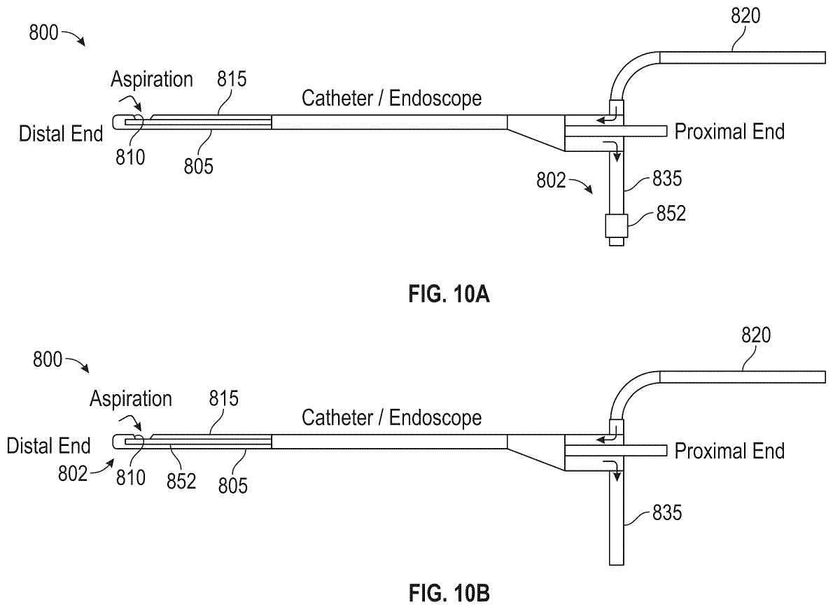

FIGS. 10A-10B, shows an instrument 800. The instrument 800 can be the same or similar to the instruments described herein, such as the instrument 100 or 200. For example, the instrument 800 can include an evacuation tube 805, one or more aspiration port openings 810, and one or more vent port openings 815. The evacuation tube 805 can be in fluid communication with a fluid supply via a first flow channel 820 and can be in fluid communication with a fluid waste channel 835. In some cases, the instrument 800 can further include an integrated sensing system 802. In some cases, the integrated sensing system 802 can include one or more sensors 852 (e.g., temperature, pressure, etc.).

FIG. 10A illustrates an example wherein the one or more sensors 852 can be positioned at a proximal side of the evacuation tube 805. For example, the one or more sensors 852 can be disposed along the fluid waste channel 835. Accordingly, the one or more sensors 852 can be configured to monitor the pressure and/or temperature of the waste fluid. Positioning the one or more sensors 852 at the proximal side of the evacuation tube 805 can result in accurate readings of the pressure within the anatomical structure (urinary tract and/or kidney (i.e., intrarenal pressure)) by intermittently starting and stopping the vacuum source. For example, the pressure at the proximal side of the evacuation tube 805 and distal to the vacuum source can have a substantially similar pressure as the pressure at the distal end of the evacuation tube 805 when the flow is paused. In some cases, positioning the one or more sensors 852 can be at a common elevation or height as the distal end of the evacuation tube 805. In some cases, positioning the one or more sensors 852 at the proximal side of the evacuation tube 805 can enable a controller to detect a formation of an obstruction or clog at the distal end of the evacuation tube 805. In some cases, positioning the one or more sensors 852 at the proximal side of the evacuation tube 805 can enable a controller to detect an imbalance in inflow and outflow from the anatomical structure.

FIG. 10B illustrates another case wherein the one or more sensors 852 can be positioned at distal side of the evacuation tube 805. In some cases, the instrument 800 can include a separate, smaller lumen e.g., to introduce and mount a pressure sensor, temperature sensor, proximity sensor, or any other sensor. In some cases, the additional lumen/channel houses the wiring, optical fibers, etc. In some cases, a signal travels through the inside or along the outside of the catheter and connects to a computer.

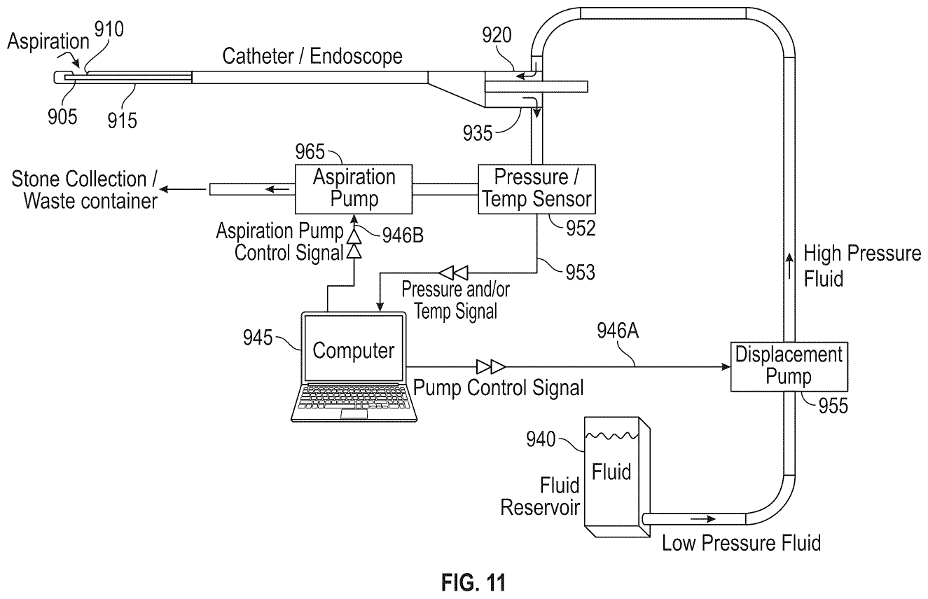

FIG. 11 shows a system 900 including inline pressure monitoring for monitoring pressure at an aspiration site and/or to detect occlusions in an aspiration line. The system 900 can include a controller 945 can be configured to adjust one or more pumps (e.g., inflow/outflow) to advantageously allow for optimal regulation of parameters and targeting of stones. The system 900 can be similar to the system 400 described herein. For example, the system 900 can include an evacuation tube 905, one or more aspiration port openings 910, one or more vent port openings 915, a liquid supply lumen 920, an evacuation lumen 935, a fluid source 940, a first control signal 946A, a second control signal 946B, one or more sensors 952, a feedback signal 953, a high-pressure fluidic pump 955, and a vacuum source 965. The one or more sensors 952 can be configured to monitor inline pressure at an aspiration site and/or detect occlusions in the evacuation lumen 935.

In some cases, the instrument can include an automated aspiration system and/or energy sources. In some cases, the automated aspiration system and/or energy sources can include a biological object engagement system that, upon detecting and/or capturing a biological object, subsequent procedures for manipulation and aspiration are triggered without manual intervention. In some cases, the automated aspiration system and/or energy sources are integrated with a sensing mechanism capable of detecting when the biological object is securely held by the catheter, prompting the automated advancement and manipulation of the energy source for object manipulation. In some cases, the sensing mechanism employs optical, ultrasonic, pressure, or tactile sensors to confirm the biological object's position and ensure its secure retention within the aspiration port.

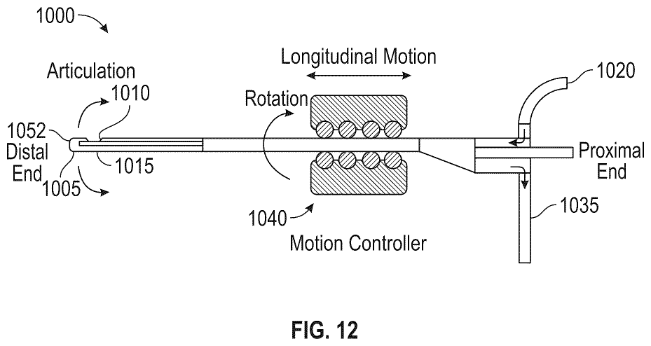

For example, as illustrated in FIG. 12, a system 1000 can include an instrument similar to those described herein, such as the instrument 100 or 200. For example, the system 1000 can include an evacuation tube 1005, one or more aspiration port openings 1010, one or more vent port openings 1015, a liquid supply lumen 1020, and an evacuation lumen 1035. In some cases, the system 1000 can further include an external motorized apparatus 1040. The external motorized apparatus 1040 can be configured to control one or both of the longitudinal and rotational movements of the catheter, as well as its articulation, whether it's of the catheter itself or the entire ureteroscope. In some cases, the system 1000 can be integrated with one or more sensors 1052 (e.g., built into the distal tip of the sheath, scope, catheter) or by computer vision (e.g., based on images received by a camera positioned proximal a distal tip), advantageously enabling it to determine the most effective way to manipulate stones. In some cases, the automated process involves advancing the evacuation tube 1005 intelligently towards a solid deposit such as a stone, securing the solid deposit to the evacuation tube 1005 via aspiration, retracting the solid deposit towards the ablation source, performing ablation on the solid deposit to break the solid deposit into fractured segments of debris, then suctioning the debris via aspiration into the evacuation lumen 1035, and, optionally, repeating the process as necessary. In some cases, computer vision (e.g., based on analyzing the endoscope video in real-time) can be used to determine the location of the debris relative to both the evacuation tube 1005 and an ablation source. In some cases, the motorized system senses, rotates, translates, and/or articulates as needed to find/attract a biological object such as a solid deposit, ablate, aspirate, and, optionally, repeat (e.g., thus enabling a complete closed-loop automated system).

In some cases, the automated aspiration system 1000 and/or energy sources can include one or more feedback loops that modulates the energy source's intensity and duration based on the size of the solid deposit, the composition of the solid deposit, and positioning data obtained from the sensing mechanism. In some cases, the automated aspiration system 1000 and/or energy sources can include a controller programmed to automate the sequence of capturing, manipulating, and aspirating the solid deposit, based on pre-defined criteria or real-time feedback.

In some cases, the automated aspiration system 1000 and/or energy sources can include one or more sensors 1052 that continuously monitor the manipulation process, adjusting the energy source parameters or pausing the process if the manipulation of the solid deposit is deemed complete or if potential complications are detected.

In some cases, the automated aspiration system 1000 and/or energy sources can be overridden or adjusted at any stage of the procedure, allowing for manual control when deemed necessary.

In some cases, the automated aspiration system 1000 can be configured to retrieve and process imaging and/or sensor feedback data to guide the energy source, ensuring optimal positioning and alignment for effective manipulation of the solid deposit.

In some cases, the automated aspiration system 1000 and/or energy sources can include one or more clearance mechanisms that, upon sensing successful manipulation and breaking down of the solid deposit, activates the aspiration process to remove fragments from the internal location.

In some cases, the automated aspiration system 1000 and/or energy sources can be configured with machine learning capabilities, allowing the automated aspiration system 1000 to adapt and enhance its engagement with solid deposits and manipulation procedures based on accumulated data from multiple procedures.

In some cases, the automated aspiration system 1000 can include a high-pressure fluid source connected to a nozzle that can operate in either a constant or pulsatile flow mode, as required for optimal engagement and manipulation of the solid deposit.

In some cases, the automated aspiration system 1000 can include a pump or a vacuum coupled to a proximal end of the evacuation lumen 1035 can be capable of operating with a constant or pulsatile flow, allowing for adaptability in aspiration based on the characteristics and size of the fragments or debris of the solid deposit.

In some cases, the automated aspiration system 1000 can be configured to be integrated with a controller that can switch between constant and pulsatile flow modes for both the fluid supply via the liquid supply lumen 1020 and the pump or vacuum connected at a proximal end of the evacuation lumen 1035, based on real-time feedback or pre-set procedural requirements.

In some cases, the automated aspiration system 1000 and/or energy sources can include a user interface to manually select between constant and pulsatile flow modes for fluid supply via the liquid supply lumen 1020 and evacuation via the evacuation lumen 1035, based on the specific needs of the procedure.

In some cases, the devices and/or systems described herein can include one or more clog detection and removal mechanisms. In some cases, the one or more clog detection and removal mechanisms include one or more sensors configured to detect obstructions and/or clogs at the one or more aspiration port openings 1015. In some cases, the sensors can be selected from the group consisting of pressure sensors, flow sensors, acoustic sensors, optical sensors, and combinations thereof.

In some cases, upon detection of a clog, the instrument can activate a clog removal mechanism utilizing the energy source to directly target and break down the clog at the aspiration port opening(s) 1015. In some cases, the clog removal mechanism can modulate the liquid jet dynamics, including altering jet pulse frequency, pressure, or flow rate, to dislodge the obstruction.

In some cases, the one or more clog detection and removal mechanisms can include a flexible wire or probe configured to be manually or automatically advanced through the evacuation lumen 1035 to mechanically dislodge or break apart the obstruction upon clog detection.

In some cases, the one or more clog detection and removal mechanisms can be configured to be integrated with a controller programmed to reverse or modulate the vacuum or peristaltic flow direction at the proximal end of the evacuation lumen 1035 in response to detected clogs.

In some cases, upon detection of a clog, the clog removal mechanism combines multiple strategies including energy application, jet modulation, and mechanical interventions to ensure comprehensive clog management.

In some cases, at least a portion of the devices and/or systems described herein can have a particular articulation and/or curvature (e.g., the evacuation tube 1005, the liquid supply lumen 1020, the evacuation lumen 1035, the sheath). In some cases, the devices can include an evacuation tube 1005 including a curved geometry, for example, to facilitate enhanced navigation and positioning within the location internal to the subject. In some cases, the devices and/or systems can include an articulation mechanism integrated with the evacuation tube 1005, enabling manual or automated bending or flexing of the evacuation tube 1005 to enhance retrieval or manipulation of the solid deposit. In some cases, the articulation mechanism consists of one or more joints, pivot points, or flex regions, allowing for multi-directional movement of the distal end of the instrument. In some cases, the articulation mechanism can enable precise orientation of the solid deposit relative to the energy source, optimizing the effectiveness of object manipulation or breakdown.

In some cases, the devices and/or systems can further include a control interface at the proximal end, permitting a user to adjust the articulation of the instrument in real-time during a procedure.

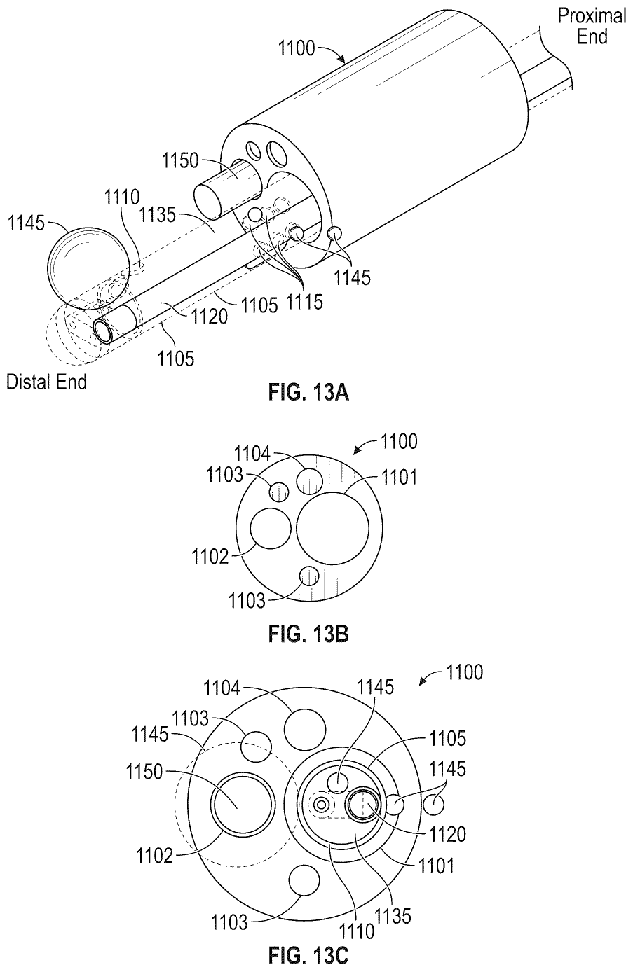

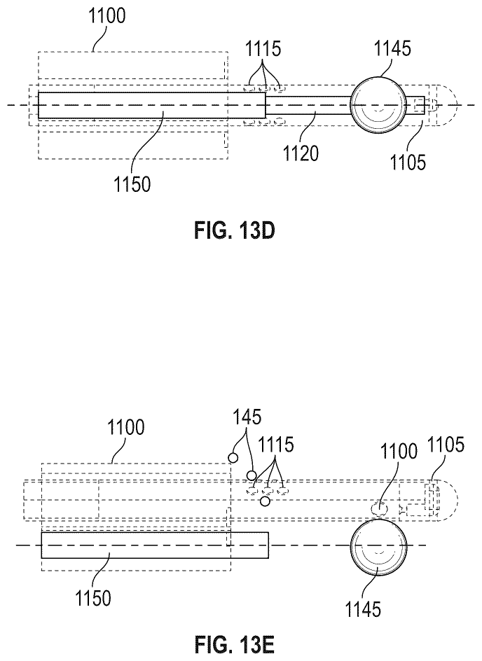

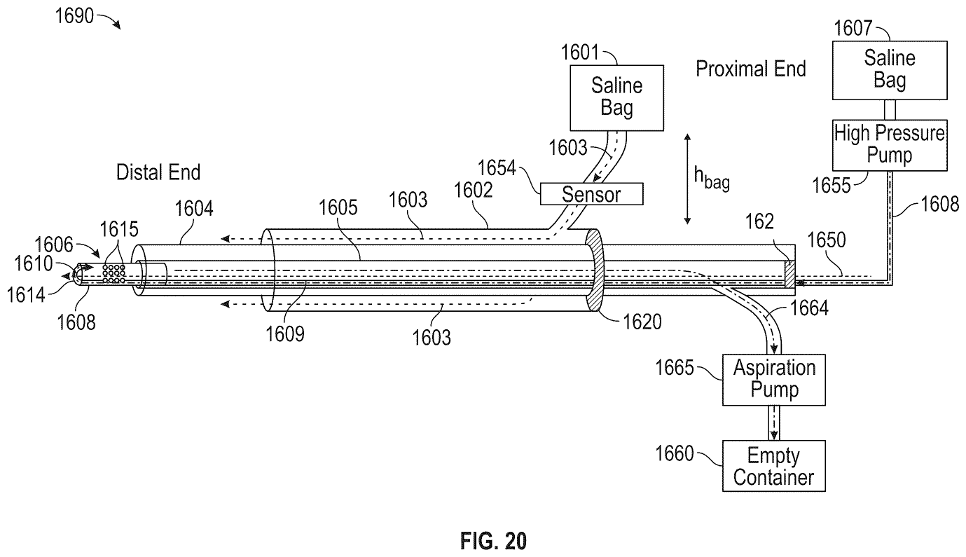

FIGS. 13A-13E illustrate a scope 1100. The scope 1100 can be a custom endoscope. However, any suitable scope known to the skilled artisan may be used as contemplated herein. The scope 1100 can include similar components as described herein. For example, the scope 1100 can include an evacuation tube 1105 (which can be similar to any of the evacuation tubes described herein) with one or more aspiration port openings 1110, one or more vent port openings 1115, a liquid supply lumen 1120, and an evacuation lumen 1135. The evacuation tube 1105 can be integrated into a custom ureteroscope. In some cases, the scope 1100 can further include an ablation instrument 1150. The ablation instrument 1150 can be an ablation tool configured to break apart and fragment a solid deposit 1145 into one or more smaller pieces (e.g., debris). As described herein, the ablation instrument 1150 can be a laser or laser fiber, an ultrasound ablation tool (such as, HIFU) or the like. For example, as shown in FIGS. 13A-13E, the ablation instrument 1150 may be a laser fiber. In some cases, an outer sheath can be provided with the custom scope 1100. A device disposed within an outer sheath may allow for simultaneous imaging, irrigation, manipulation of the solid deposit 1145, and aspiration during procedures, according to some cases. In some cases, integrating the devices and/or systems disclosed herein with the scope 1100 can provide synchronized control or articulating mechanisms, allowing simultaneous operation of imaging, aspiration, irrigation, and object manipulation. In some examples, a space between an outer surface of the scope and an inner surface of the outer sheath may provide an irrigation flow, for example as described in greater detail herein with reference to FIG. 20. In some examples, the outer sheath may be integrated with the scope 1100. For example, an irrigation channel may be disposed within the scope 1100 for providing an irrigation fluid into the anatomical structure.

FIG. 13A shows a perspective view of the scope 1100. As shown in FIG. 13A, the evacuation tube 1105 can extend distally from the scope 1100. The evacuation tube 1105 can be configured to aspirate one or more solid deposits 1145. The one or more solid deposits 1145 can include large solid deposits that are too large to pass through the one or more aspiration port openings 1110 and/or through the evacuation lumen 1135 and small solid deposits that can pass through the one or more aspiration port openings 1110 and/or through the evacuation lumen 1135. In some cases, the one or more aspiration port openings 1110 may secure the large solid deposits 1145 in place for the ablation instrument 1150 to ablate and fracture the large solid deposit 1145 into fragments of debris. The ablation instrument 1150 can be positioned laterally offset from the longitudinal axis of the evacuation tube 1105 and aligned with the solid deposit 1145. In some cases, the ablation instrument 1150 can have diameter of about 0.7 mm.

FIG. 13B shows a front view of the scope 1100. The scope 1100 can include a plurality of openings extending through the scope 1100. In some cases, the plurality of openings can include a first opening 1101 and a second opening 1102. The first opening 1101 can be sized to receive the evacuation tube 1105. For example, the dimensions of the first opening 1101 can correlate to the outer dimensions of the evacuation tube 1105. In some cases, the first opening 1101 can be sized larger than the outer dimensions of the evacuation tube 1105. For example, the diameter of the first opening 1101 can be about 1.4 mm (4.2 French). The second opening 1102 can be sized to receive the ablation instrument 1150. For example, the dimensions of the second opening 1102 can correlate to the outer dimensions of the ablation instrument 1150. In some cases, the second opening 1102 can be sized larger than the outer dimensions of the ablation instrument 1150. For example, the diameter of the second opening 1102 can be about 0.8 mm (2.4 French). The scope 1100 can further include one or more lighting elements 1103. The one or more lighting elements 1103 can be configured to provide light into the urinary system of a subject. For example, the one or more lighting elements 1103 can be light-emitting diodes (LED). In some cases, the one or more lighting elements 1103 can be distal facing and/or positioned on a distal face of the scope 1100. The scope 1100 can further include an optical element 1104. In some cases, the optical element 1104 can be configured to provide an optical view of the distal end of the scope 1100 to a physician. For example, the optical element 1104 can be a camera.

FIG. 13C shows another front view of the scope 1100 with the evacuation tube 1105, a plurality of solid deposits 1145, and the ablation instrument 1150. As shown in FIG. 13C, a solid deposit 1145 can be secured to the evacuation tube 1105 via aspiration at the one or more aspiration port openings 1110. The one or more aspiration port openings 1110 can be positioned on a side of the evacuation tube 1105 facing the ablation instrument 1150. In some cases, the longitudinal axis of the ablation instrument can be parallel to the longitudinal axis of the evacuation tube 1105. Accordingly, the ablation instrument 1150 can be configured to ablate and/or fragment the solid deposit 1145 secured to the side of the evacuation tube 1105. As shown in FIG. 13C, the first opening 1101 can be larger than the evacuation tube 1105. In some cases, the space between the first opening 1101 and the outer dimension of the evacuation tube 1105 can provide an irrigation fluid. For example, an irrigation fluid such as a saline solution can be provided into the urinary system of the subject via the space between the first opening 1101 and the evacuation tube 1105. The irrigation fluid can be used to inflate and/or expand organs of the urinary system.

FIG. 13D shows a side elevation view of the scope 1100 with the evacuation tube 1105, a solid deposit 1145, and the ablation instrument 1150. As shown in FIG. 13D, the longitudinal axes of the evacuation tube 1105 and the ablation instrument 1150 can be co planar. The longitudinal axis of the ablation instrument 1150 can intersect the solid deposit 1145. Accordingly, the ablation instrument 1150 can emit energy along its longitudinal axis to break up and fragment the solid deposit 1145 into debris.

FIG. 13E shows a top view of the scope 1100 with the evacuation tube 1105, a plurality of solid deposits 1145, and the ablation instrument 1150. As shown in FIG. 13E, the longitudinal axis of the ablation instrument 1150 can intersect a solid deposit 1145 secured to the evacuation tube 1105 via the one or more aspiration port openings 1110.

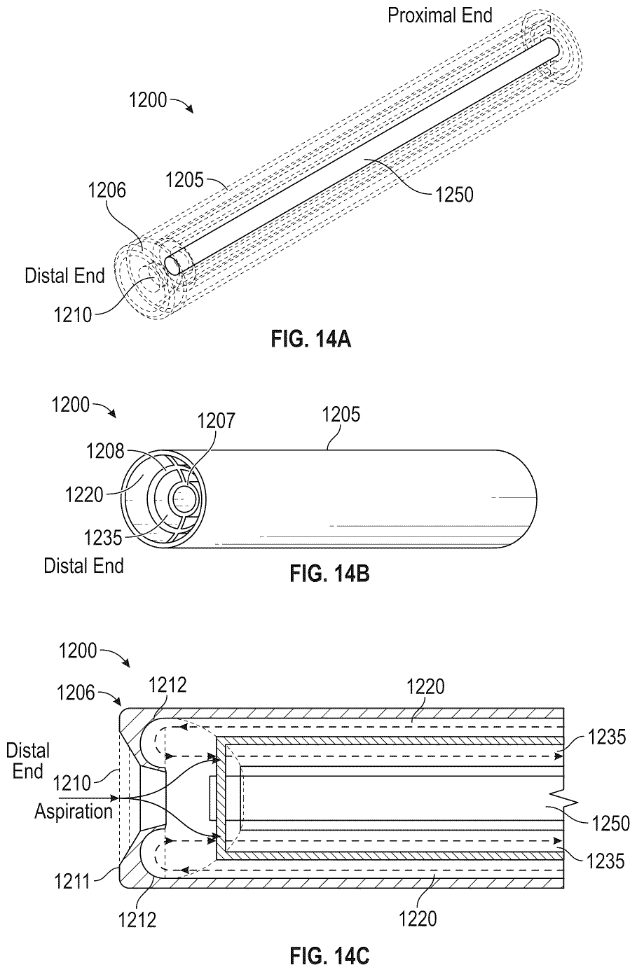

FIGS. 14A-14C illustrate an instrument 1200 having a concentric design. The instrument 1200 can be similar to any of the instruments described herein, such as the instruments 100 or 200, except for the differences described herein. In some cases, the instrument 1200 can include a concentric device design for energy delivery and aspiration at the distal end.

In some cases, the instrument 1200 can include an evacuation tube 1205 having a plurality of radially nested inner tubes. For example, the evacuation tube 1205 can include at least one inner tube. As shown in FIGS. 14A-14C, the evacuation tube 1205 can have a first inner tube 1207 and a second inner tube 1208. The first inner tube 1207 can be radially nested and disposed within the second inner tube 1208. The second inner tube 1208 can be radially nested and disposed within an outer body portion of the evacuation tube 1205. Accordingly, the evacuation tube 1205 can include a plurality of longitudinal volumes extending from a proximal end to a distal end.

As shown in FIGS. 14A-14C, the plurality of volumes can include a first interior volume, a second interior volume and a third interior volume. The first volume can be defined by the walls of the first inner tube 1207. In some cases, the first volume can be generally defined by the cross-sectional area and the length of the first inner tube 1207. For example, the first volume can be defined as: πr₁₂₀₇² L₁₂₀₇, wherein r₁₂₀₇ is the radius of the first inner tube 1207 and L₁₂₀₇ is the length of the first inner tube 1207. The second volume can be defined by the space between the walls of the second inner tube 1208 and the first inner tube 1207. In some cases, the second volume can be generally defined by the cross-sectional area of the second inner tube 1208, the length of the second inner tube 1208, the cross-sectional area of the first inner tube 1207, and the length of the first inner tube 1207 less any support structures. For example, the second volume can be defined as: (πr₁₂₀₈² L₁₂₀₈)−(πr₁₂₀₇² L₁₂₀₇)−n t Δr L₁₂₀₈, wherein r₁₂₀₈ is the radius of the second inner tube 1208, L₁₂₀₈ is the length of the second inner tube 1208, r₁₂₀₇ is the radius of the first inner tube 1207, L₁₂₀₇ is the length of the first inner tube 1207, n is the number of support structures, t is the thickness of the support structures, and Ar is the radial distance between the first inner tube 1207 and the second inner tube 1208. The third volume can be defined by the space between the walls of the outer body portion of the evacuation tube 1205 and the walls of the second inner tube 1208. In some cases, the third volume can be generally defined by the cross-sectional area of the evacuation tube 1205, the length of the evacuation tube 1204, the cross-sectional area of the second inner tube 1208, and the length of the second inner tube 1208 less any support structures. For example, the third volume can be defined as: (πr₁₂₀₅² L₁₂₀₅)−(πr₁₂₀₈² L₁₂₀₈)−n t Δr L₁₂₀₅, wherein r₁₂₀₅ is the radius of the evacuation tube 1205, L₁₂₀₅ is the length of the evacuation tube 1205, r₁₂₀₈ is the radius of the second inner tube 1208, L₁₂₀₈ is the length of the second inner tube 1208, n is the number of support structures, t is the thickness of the support structures, and Ar is the radial distance between the second inner tube 1208 and the evacuation tube 1205.

The plurality of longitudinal volumes can be configured to function as a liquid supply lumen, an evacuation lumen or aspiration lumen, and/or an access lumen for providing an ablation instrument 1250. For example, one of the plurality of volumes can be in fluid communication with an aspiration source such as a vacuum or aspiration pump. In some cases, the volume in fluid communication with an aspiration source can be an evacuation lumen 1235. For example, the second volume may be an evacuation lumen 1235. A different one of the plurality of volumes can be in fluid communication with a fluid source and may be configured to receive a pressurized fluid. In some cases, the volume in fluid communication with the fluid source can be a liquid supply lumen 1220. For example, the third volume may be a liquid supply lumen 1220. A different one of the plurality of volumes can be configured to receive an ablation instrument 1250. For example, the first volume may be a lumen configured to receive an ablation instrument 1250.

The instrument 1200 can further include an optional inverter cap 1206. The inverter cap 1206 can be configured to redirect a fluid flow toward the evacuation tube 1205. As described herein, the inverter cap 1206 can be configured to redirect a fluid flow 180 degrees. In some cases, the inverter cap 1206 can have an annular structure with a central lumen extending therethrough. In some implementations, a liquid supply lumen 1220 can be shaped so as to redirect the fluid flow toward the proximal end (see, for instance, FIG. 3A) without the use of the inverter cap 1206.

The central lumen can include an aspiration port opening 1210. As shown in FIGS. 14A-14C, the aspiration port opening 1210 can be positioned at a distal face of the instrument 1200. In some cases, the aspiration port opening 1210 can be a funnel shape wherein the aspiration port opening 1210 is larger on the distal side than the proximal side. The inverter cap 1206 can include a first surface 1211 and a second surface 1212 opposite the first surface 1211. In some cases, the first surface 1211 be positioned on a distal end of the inverter cap 1206 and the second surface 1212 can be on a proximal end of the inverter cap 1206. The second surface 1212 can include an annular groove extending annularly along the second surface 1212. The annular groove can be rounded. For example, the annular groove can be a semi-circle. Accordingly, a fluid entering the second surface 1212 can be redirected by the annular groove. The inverter cap 1206 can be coupled to the distal end of the evacuation tube 1205. In some cases, the annular groove can be axially displaced from the second volume and the third volume of the evacuation tube 1205. For example, the annular groove of the second surface 1212 can be axially displaced by a distance X. Accordingly, a fluid flow from one of the second or third volumes can be redirected into the other of the second or third volumes. For example, a fluid flow from the liquid supply lumen 1220 can be redirected by the annular groove of the second surface 1212 into the evacuation lumen 1235. The axial displacement can also allow external fluid and solid deposits to be aspirated through the evacuation lumen 1235.

The instrument 1200 can further include an ablation instrument 1250. In some cases, the ablation instrument 1250 can be housed within the first inner tube 1207. The ablation instrument 1250 can be an ablation tool configured to break apart and fragment a solid deposit into one or more smaller pieces (e.g., debris). As described herein, the ablation instrument 1250 can be a laser or laser fiber, an ultrasound ablation tool (such as, HIFU) or the like. For example, as shown in FIGS. 14A-14C, the ablation instrument 1250 may be a laser fiber. Accordingly, the first volume defined by the wall of the first inner tube 1207 can be filled by the ablation instrument 1250 configured to fragment a solid deposit. In some cases, the ablation instrument 1250 can emit energy through the first inner tube 1207 while the second inner tube 1208 provides aspiration thereby facilitating simultaneous energy delivery and aspiration.

In some cases, the radially nested tubes can include an outer tube and an inner tube. The outer tube can be similar to the outer body portion of the evacuation tube 1205 and the inner tube can be the same as the second inner tube 1208 described above. Accordingly, the evacuation tube 1205 can be similar to the evacuation tube 1205 described above without the first inner tube 1207. In such embodiments, the plurality of volumes can include an inner volume and an outer volume. The inner volume can be generally defined by the cross-sectional area of the inner tube and the length of the inner tube. For example, the outer volume can be defined as: π rᵢₙₙₑᵣ² Lᵢₙₙₑᵣ, wherein rᵢₙₙₑᵣ is the radius of the inner tube and Lᵢₙₙₑᵣ is the length of the inner tube. The outer volume can be generally defined by the cross-sectional area of the evacuation tube 1205, the length of the evacuation tube 1204, the cross-sectional area of the inner tube, and the length of the inner tube less any support structures. For example, the outer volume can be defined as: (π rₒᵤₜₑᵣ² Lₒᵤₜₑᵣ)−(π rᵢₙₙₑᵣ² Lᵢₙₙₑᵣ)−n t Δr, wherein rₒᵤₜₑᵣ is the radius of the evacuation tube 1205, Lₒᵤₜₑᵣ is the length of the evacuation tube 1205, Fᵢₙₙₑᵣ is the radius of the inner tube, Lᵢₙₙₑᵣ is the length of the inner tube, n is the number of support structures, t is the thickness of the support structures, and Ar is the radial distance between the inner tube and the evacuation tube 1205.

The effective cross-sectional areas of the volumes within the evacuation tube 1205 can be different. In some cases, the effective cross-sectional area of the volume between the evacuation tube 1205 and an inner tube can be less than the effective cross-sectional area of the inner tube. For example, the third volume described above can have an effective cross-sectional area of: (πr₁₂₀₅²)−(π r₁₂₀₈²)−n t Δr and the second volume described above can have an effective cross-sectional area of (π r₁₂₀₈²)−(π r₁₂₀₇²)−n t Δr, wherein r₁₂₀₅² r₁₂₀₈² is less than r₁₂₀₈²−r₁₂₀₇². Additionally or alternatively, the outer volume described above can have an effective cross-sectional area of: (π rₒᵤₜₑᵣ²)−(π rᵢₙₙₑᵣ²)−n t Δr and the inner volume described above can have an effective cross-sectional area of: (π rₒᵤₜₑᵣ²)−(π rᵢₙₙₑᵣ²), wherein rₒᵤₜₑᵣ²−Fᵢₙₙₑᵣ² is less than rᵢₙₙₑᵣ². Accordingly, a flow rate passing from the third volume and/or outer volume into the second volume and/or inner volume passes from a constricted lumen to an expanded lumen. In such cases, a supply lumen can include a first effective cross-sectional area and an evacuation lumen and/or aspiration lumen (the term evacuation lumen may be interchangeable with the term aspiration lumen) can include a second effective cross-sectional area greater than the first effective cross-sectional area, wherein a flow of liquid can be ejected from a volume of the supply lumen with the first effective cross-sectional area into a volume of the catheter body with the second effective cross-sectional area.

In some cases, the concentric design of the instrument 1200 illustrated in FIGS. 14A-14C can be modified such that the ablation instrument 1250 can emit energy through the second inner tube 1208 and the aspiration can be facilitated through the first inner tube 1207, thereby enabling the solid deposit to be suctioned, retained, and cleared from the distal end of the evacuation tube 1205 while the ablation instrument 1250 is active. Additionally or alternatively, the concentric design of the instrument 1200 illustrated in FIGS. 14A-14C can be modified to include only two radially nested tubes including an inner tube and an outer tube as described above. In such cases, the ablation instrument may be positioned in the inner volume or the outer volume.

The concentric design of the instrument 1200 can offer multiple advantages. The concentric design of the instrument 1200 can allow for an uninterrupted energy source operation while the aspiration process captures, retains, and clears solid deposits. In some cases, clearance of fragments, debris, and dust can be enhanced by applying vacuum at the site of energy application near the aspiration port opening. For example, a high velocity flow from the liquid supply lumen into the evacuation lumen and/or aspiration lumen can be provided at the site of energy application as shown in FIGS. 14A-14C. Additionally, temperatures due to the ablation instrument 1250 may be reduced by passing the fluid flow from the liquid supply lumen into the evacuation lumen proximally to (such as, in front of) the ablation instrument 1250. The concentric design of the instrument 1200 can provide a liquid supply lumen, an evacuation lumen and/or aspiration lumen, and an ablation instrument 1250 within a single working channel of a ureteroscope. Accordingly, the concentric design of the instrument 1200 may be compatible with existing ureteroscopes having a single working channel. Coupling the discrete lumens in the concentric configuration can ease manipulation of the instrument 1200 by controlling a single assembly rather than managing separate components. Furthermore, positioning the ablation instrument 1250 at the aspiration port opening can minimize clogging and the risk of obstructing the evacuation lumen since fragments and debris cannot pass around the ablation instrument 1250 until they are small enough to fit through the gap at the distal end of the instrument 1200.

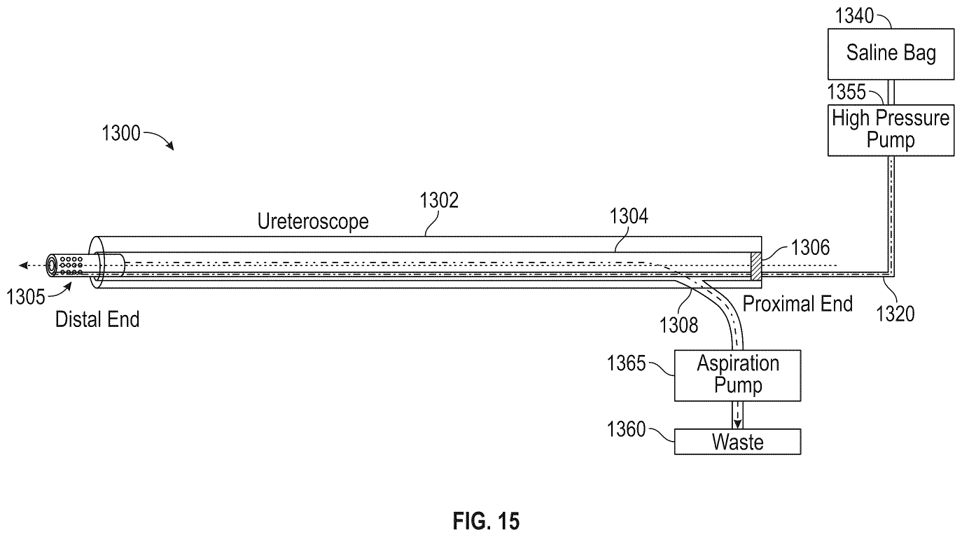

FIG. 15 shows a schematic of a ureteroscope system 1300, which can be similar to any of the systems described herein. While the system 1300 is described in the context of ureteroscopy, the system 1300 or any of the systems described herein can be used for any medical procedure that utilizes a catheter.

The ureteroscope system 1300 can include a ureteroscope 1302. The ureteroscope 1302 can include on or more lumens 1304. The ureteroscope 1302 can include one or more inlets 1306 and one or more outlets 1308 in fluid communication with the one or more lumens 1304. In some examples, the one or more inlets 1306 can be configured to deliver energy emitted from an ablation instrument and/or a liquid supply lumen 1320 in fluid communication with a fluid source 1340 via a high-pressure fluidic pump 1355. In some examples, the one or more inlets 1306 may be sealed to prevent egress from the one or more lumens 1304. The one or more outlets 1308 can be in fluid communication with the one or more lumens 1304. In some examples, the one or more outlets 1308 can be configured to provide an opening to evacuate from the ureteroscope 1302 waste material to a waste container 1360. The waste material can include biological objects. In some examples, the ureteroscope system 1300 can include an aspiration pump 1365 to assist with aspirating the waste material to the waste container 1360.

The ureteroscope system 1300 can further include an evacuation tube 1305. The evacuation tube 1305 can be any of the evacuation tubes described herein, such as the evacuation tube 105 or 205. For example, the evacuation tube 1305 can have a concentric design as described with regards to FIGS. 14A-14C.

The ureteroscope 1302 can be introduced to a portion of a subject's urinary system. For example, the ureteroscope 1302 can be introduced to one of the subject's kidneys. The high-pressure fluidic pump 1355 can be activated to provide a high flow rate of fluid flow through the liquid supply lumen 1320 to the distal end of the evacuation tube 1305. The evacuation tube 1305 can redirect the fluid from the liquid supply lumen 1320 toward an evacuation lumen within the one or more lumens 1304.

As described herein, the redirection of fluid can induce a Venturi effect to provide a low pressure at the distal end of the ureteroscope 1302. While aspiration pump 1365 can provide suction to the proximal end of the ureteroscope 1302, the additional suction provided by the liquid jet at the proximal end of the ureteroscope 1302 can facilitate consistent maintenance of a suction level necessary to capture a kidney stone (or more generally a biological object) at or near the distal tip of the evacuation tube 1305 and allow for efficient and safe removal the kidney stone. Energy from an ablation source can be used to fracture the kidney stone into debris that are aspirated through the one or more lumens 1304 into the waste container 1310.

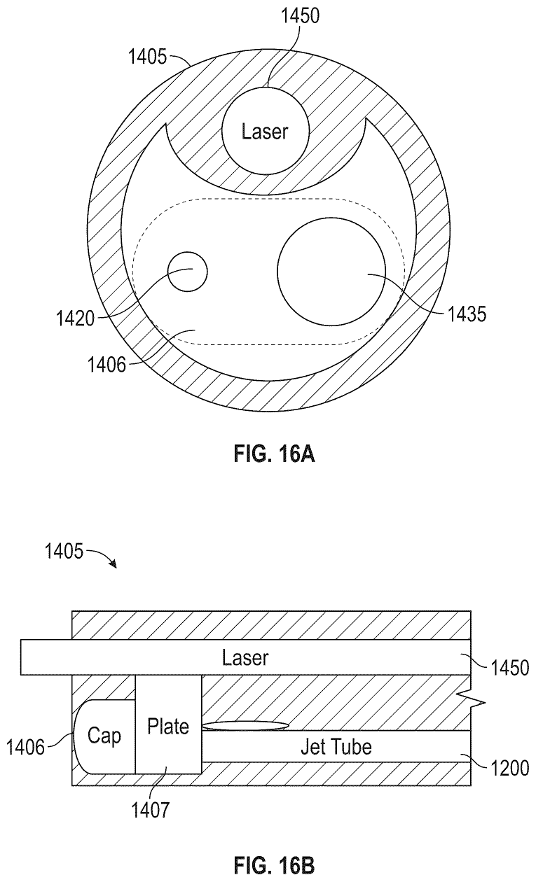

FIGS. 16A-16B illustrate an evacuation tube 1405 with an ablation instrument (e.g., a laser fiber) disposed within the evacuation tube 1405 itself. The evacuation tube 1405 can be part of a custom endoscope. The evacuation tube 1405 can be similar to any of the evacuation tubes described herein. For example, the evacuation tube 1405 can include one or more aspiration port openings, one or more vent port openings, a liquid supply lumen 1420, and an evacuation lumen 1435. In some examples, the evacuation tube 1405 can further include an ablation instrument 1450. The ablation instrument 1450 can be an ablation tool configured to break apart and fragment a solid deposit into one or more smaller pieces (e.g., debris). As described herein, the ablation instrument 1450 can be a laser or laser fiber, an ultrasound ablation tool (such as, HIFU) or the like. Integrating an ureteroscope as described herein with the evacuation tube 1405 can provide synchronized control or articulating mechanisms, allowing simultaneous operation of imaging, aspiration, irrigation, and object manipulation.

FIG. 16A shows a front view of the evacuation tube 1405. As shown in FIG. 16A, the evacuation tube 1405 can include a liquid supply lumen 1420 and an evacuation lumen 1435. The liquid supply lumen 1420 can be the same or similar to the liquid supply lumen s described herein. The evacuation lumen 1435 can be the same or similar to the evacuation lumens described herein. The evacuation tube 1405 can further include an inverter cap 1406. The inverter cap 1406 can be configured to redirect fluid flow from the liquid supply lumen 1420 toward the evacuation lumen 1435.

FIG. 16B shows a side cross-sectional view of the evacuation tube 1405. As shown in FIG. 16B, the evacuation tube 1405 can further include an inverter plate 1407. The inverter plate 1407 can have an annular structure with one or more openings extending therethrough. The one or more openings can be configured to receive and/or be part of the liquid supply lumen and/or the evacuation lumen 1435.

Accordingly, in some cases, the ablation instrument 1450 described herein (e.g., a laser) can be disposed within a portion of the evacuation tube 1405 and/or evacuation lumen 1435. In some cases, the ablation instrument 1450 may be manipulated (e.g., via a motorized apparatus) to move and/or rotate within the evacuation tube 1405. In some cases, the ablation instrument 1450 may be retracted (e.g., behind the inverter cap 1406 and/or inverter plate 1407 relative to the distal end of the evacuation tube 1405) such that a liquid may flow through an opening in which the ablation instrument 1450 was inserted. In some cases, the opening configured to receive the ablation instrument 1450 can be converted to an aspiration port opening upon retraction of the ablation instrument 1450 within the evacuation tube 1405.

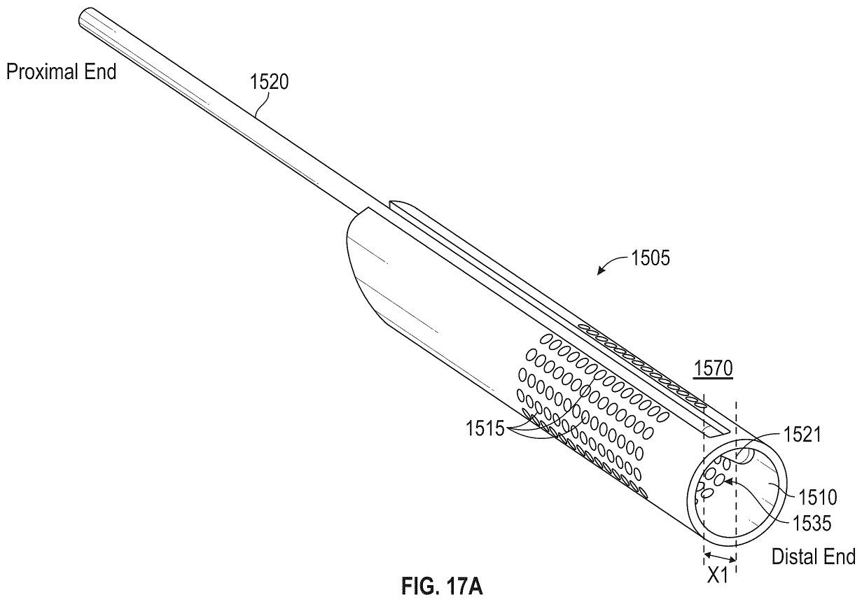

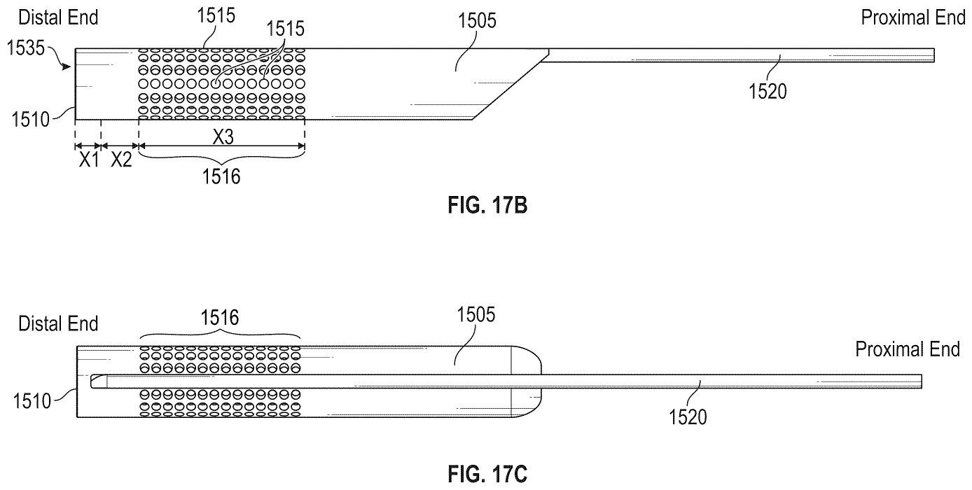

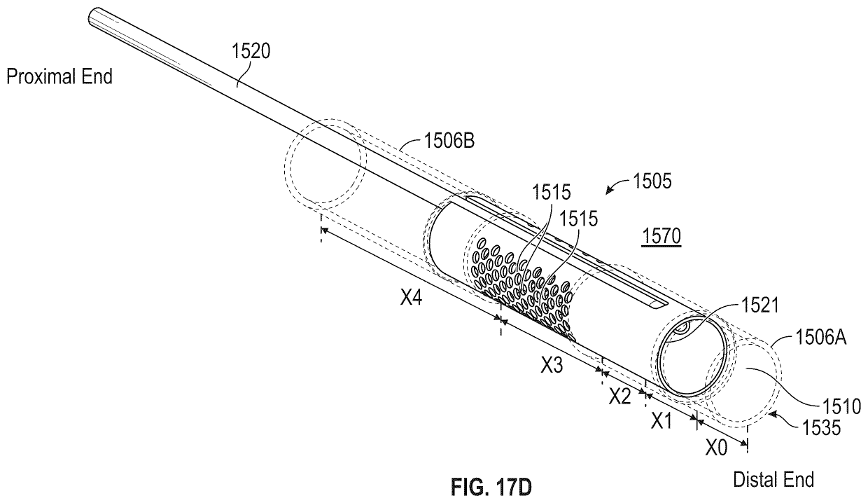

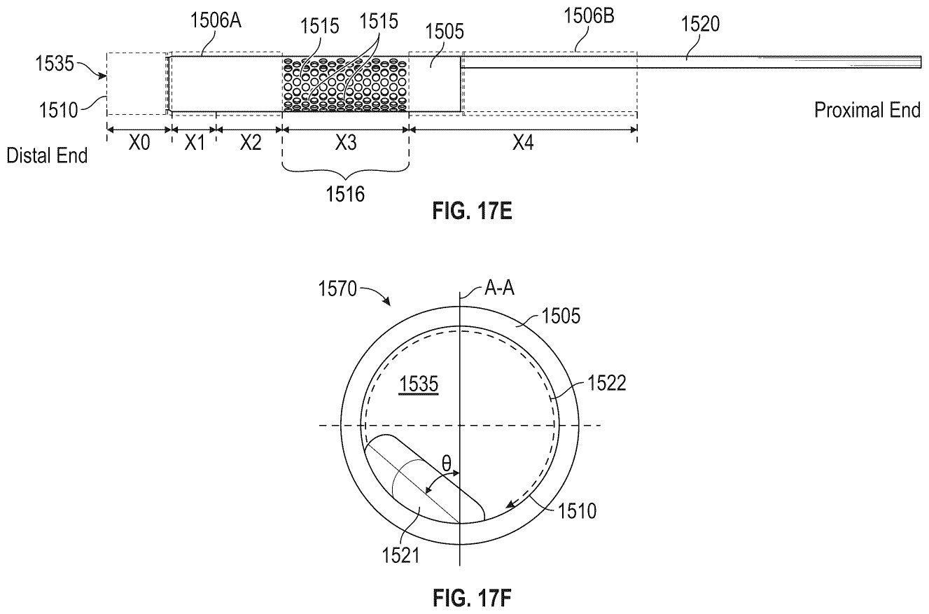

FIGS. 17A-17C illustrate an evacuation tube 1505 similar to any of the evacuation tubes described herein with the exception of the evacuation tube 1505 having an angled J-tube bend. The evacuation tube 1505 can include an aspiration port opening 1510, a plurality of vent port openings 1515 (only a couple of the plurality of vent port openings 1515 are labeled as examples), a liquid supply lumen 1520, and an evacuation lumen 1535. The evacuation tube 1505 can be disposed at a distal end of the liquid supply lumen 1520. Accordingly, the evacuation tube 1505 can be inserted through a ureteroscope and extend at least distally past the distal end of the ureteroscope. As shown in FIGS. 17A-17C, the aspiration port opening 1510 can be positioned at a distal face of the evacuation tube 1505.

FIG. 17A illustrates a perspective view of the evacuation tube 1505. As shown in FIG. 17A, the evacuation tube 1505 can include one aspiration port opening 1510. The liquid supply lumen 1520 can provide a fluid from a fluid source. For example, the liquid supply lumen 1520 can be the same or similar to the liquid supply lumens described herein. In some examples, the liquid supply lumen 1520 can include a J-tube bend 1521 at the distal end of the evacuation tube 1505. The J-tube bend 1521 redirects the fluid flow through the liquid supply lumen 1520. For example, the J-tube bend 1521 can redirect the fluid flow from a distal flow direction to a proximal flow direction. In some examples, the J-tube bend 1521 can include a constriction (such as, a nozzle with a constriction) for forming a liquid jet. In some cases, the constriction can extend along the length of the liquid supply lumen 1520. In such cases, the liquid supply lumen 1520 can have a constant inner diameter defining the constriction as a step down from a proximal supply lumen extending between a fluid source and the liquid supply lumen 1520. For example, the proximal supply lumen can be a tube (for example, made from plastic) extending from the fluid source to the proximal end of the liquid supply lumen 1520. The inner diameter of the proximal supply lumen can be greater than an inner diameter of the liquid supply lumen 1520. Accordingly, the liquid supply lumen 1520 can define a step-down or constriction for the liquid flowing through the liquid supply lumen 1520. In some cases, the liquid supply lumen 1520 can have a constant inner diameter such that the entire liquid supply lumen 1520 defines the constriction. In some cases, the liquid supply lumen 1520 can include a nozzle positioned at a distal end of the J-tube bend 1521. For example, the constriction can be variable within the J-tube bend 1521, wherein the nozzle is configured to taper the constriction toward an outlet. Accordingly, pressure at the distal end of the evacuation tube can be reduced as a result of the Venturi effect, which can facilitate with maintaining consistent suction at the distal end of the evacuation tube 1505 for removal of biological objects, as described herein.

Without wishing to be bound by any particular theory, a jet pump (or eductor pump) may be used to draw fluid into the instrument, device, and/or system. A high-speed jet of a fluid issuing into a second fluid can be configured to entrain a second fluid into the jet. A fluid jet may be configured to entrain fluid from the internal body part into the catheter, providing suction at the distal tip of the catheter. A jet pump can operate as a fluid flow amplifier that moves fluids using a high-velocity fluid stream as the driving force. This operation can be based on the Venturi effect, by which the driving fluid creates a low-pressure area that sucks in and entrains a secondary fluid to be pumped. The high-velocity fluid stream may have a smaller flow rate than that of the secondary fluid.

In some examples, the J-tube bend 1521 can include a constant inner dimension with the liquid supply lumen 1520. In such embodiments, the liquid flowing through the liquid supply lumen 1520 can have a constant high flow rate. For example, a high-pressure pump as described herein with respect to FIGS. 6A-6F, 11, and 15 can provide a constant high flow rate of liquid through the liquid supply lumen 1520.

An entrainment effect (also referred to as entrainment) and/or a jet-pump effect can explain the movement of low velocity or still fluid, along with any particles conveyed by the fluid, towards a high velocity fluid jet. Outputting a high velocity flow from a small orifice or nozzle exit into a low velocity body of fluid tends to draw the low velocity into the high-speed jet. Liquids can exist within the patient. For example, the patient's urinary system can have one or more preexisting liquids including urine, blood, and/or other bodily fluids. The systems described herein can introduce additional liquids to the patient's urinary system. For example, the systems described herein can introduce a saline solution to irrigate and/or flush biological objects including blood clots, tumors, tissue samples, and urinary or fragmented urinary calculi such as bladder stones, ureter stones, and kidney stones from the patient. These liquids can experience no-slip conditions. Under no-slip conditions, liquids immediately adjacent to a solid surface cannot move relative to the solid surface. Accordingly, liquids immediately adjacent to a solid surface have zero velocity. Similarly, adjacent liquid molecules can experience no-slip conditions such that the velocity of one liquid molecule can affect adjacent molecules. For example, the shear stress at the interface of the high-speed jet and the lower velocity surrounding entrains, or draws, the fluid into the jet. As fluid flows toward the high-speed jet by entrainment, the drag force acting on the particles or biological objects in the surrounding fluid attracts the particles toward the high-speed jet.

The jet-pump effect can correspond to the creation of a vacuum by introducing a first liquid body having high-flow rate as a liquid jet into a second liquid body having a low flow rate. The no-slip conditions, friction, and entrainment between adjacent liquid molecules of the jet from the first liquid body and the second liquid body can pull the molecules of the second liquid body into the flow path of the jet of the first liquid body. Accordingly, energy and/or velocity vectors of the jet from the first liquid body can be dissipated through friction as it travels farther within the second liquid body by introducing the jet from the first liquid body having a high flow rate into the second liquid body having lower flow rate. This dissipation of the energy and/or velocity vectors of the jet from the first liquid body is accompanied by entrainment of fluid from the second liquid body. In some cases, the difference between the relative flow rates can affect the quantity or volume of the second liquid body that is induced to follow the flow path of the first flow path. The greater the difference—or the higher the flow rate of the first liquid body relative to the second liquid body—can result in more energy and/or velocity being dissipated throughout the second liquid body.

Furthermore, introducing a small volume of a first liquid body having a high flow rate into a large volume of a second liquid body having a low flow rate can result in moving a greater volume of liquid. Accordingly, higher flow rates of the first liquid body can result in inducing a greater quantity of the second liquid body to follow the flow direction of the first liquid body. In some cases, a liquid supply lumen can include a first diameter and an evacuation lumen can include a second diameter greater than the first diameter, wherein a flow of a liquid can be ejected from a volume of the liquid supply lumen with the first diameter into a volume of the evacuation lumen with the second diameter. In some cases, an evacuation tube can be configured to create vacuum using both Venturi-effect and entrainment/jet-pump effect.

This entrainment or jet pump effect creates pressure gradients within the second liquid body with lower pressures near the high velocity jet issuing from the first liquid body. The high velocity of the jet issuing from the first liquid velocity and the increased velocity of the second liquid body at the interface with the jet from the first liquid body can decrease the corresponding pressure thereby creating a vacuum. The entrainment effect is also shown in FIGS. 14A-14C.

As shown in FIG. 17A, the aspiration port opening 1510 can face the distal end of the evacuation tube 1505. Accordingly, the evacuation tube 1505 can capture a solid deposit at or near its distal end, facilitate breaking up the solid deposit into fragments by an ablation device, and aspirate the fragments.

The plurality of vent port openings 1515 can be positioned annularly around the evacuation tube 1505. The plurality of vent port openings 1515 can be configured to regulate pressure, regulate maximum suction performance, regulate the proportion of egress flow relative to the percent of ingress flow, regulate the size of debris and fragments aspirated within the evacuation lumen 1535 (prevent clogging), and to generate a swirling or helical motion that can agitate adjacent biological objects thereby increasing the changes of inducing the adjacent biological objects to enter a suction field. In some examples, fluids can flow between an interior volume of the evacuation tube 1505 and an exterior volume 1570 through the plurality of vent port openings 1515. In some examples, fluids can simultaneously egress and ingress through the plurality of vent port openings 1515 at different locations based on the local pressure differences between the interior volume of the evacuation tube 1505 and the exterior volume 1570.

FIGS. 17B-17C illustrate side and top views of the evacuation tube 1505, respectively. As shown in FIGS. 17B-17C, the plurality of vent port openings 1515 can be disposed completely around the annular structure of the evacuation tube 1505. In some examples, the outlet of the J-tube bend 1521 can be axially displaced from the distal end of the evacuation tube 1505 by an axial distance X1. The axial distance X1 can be less than 3 mm. In some cases, the axial distance X1 can be about 1.9 mm. In some examples, the plurality of vent port openings 1515 can be axially displaced from the outlet of the J-tube bend 1521 by an axial distance X2. The axial distance X2 can be less than 3 mm. In some examples, the axial distance X2 can be 1.7 mm. In some cases, the combined axial distance of X1 and X2 can be less than 5 mm. For example, the combined axial distance of X1 and X2 can be between about 0.1 mm to 5 mm. The axial distance X2 can reduce the flow rate of the fluid from a first velocity at the exit of the J-bend tube and the distal end of the vent port openings 1515. Additionally or alternatively, the axial distance X2 can provide a solid wall in the exhaust path of the liquid jet. This can advantageously prevent the liquid jet from egressing directly into the external environment. Accordingly, the liquid jet can substantially remain within the evacuation tube 1505. Increasing the axial distance X2 can enhance the effective suction within the evacuation tube 1505 because the fluid is further dissipated before the liquid jet reaches the plurality of vent port openings 1515. In some cases, a secondary Venturi effect can be created at the plurality of vent port openings 1515 if the velocity flow from liquid supply lumen 1520 is too fast. In such cases, the secondary Venturi effect can cause a reverse flow through the aspiration port opening 1510 at the distal end of the evacuation tube 1505. Accordingly, increasing the axial X2 can dissipate and slow the liquid jet to prevent a secondary Venturi effect at the plurality of vent port openings 1515. The plurality of vent port openings 1515 can be disposed in a vent port section 1516 of the evacuation tube 1505. The vent port section 1516 can extend along an axial length X3 of the evacuation tube 1505. The axial length X3 can provide for fluid balancing between the external environment and the evacuation tube 1505. In some cases, the axial distance X3 can be long enough to allow fluid to exit the evacuation tube 1505 into the external environment when the pressure within the evacuation tube 1505 exceeds the external environment and vice versa. For example, the axial distance X3 can be about 2.1 mm. In some cases, performance can be enhanced by increasing the vent port section 1516. For example, increasing the axial distance X3 can increase the quantity of vent port openings 1515 which can increase the amount of liquid that can circulate through the distal opening of the evacuation tube 1505 and out of the vent port openings 1515. In such cases, the increased quantity of vent port openings 1515 can result in enhanced fluid velocity and suction.

In some cases, the axial distances X1, X2, and X3 can be determined by balancing various factors including fluid flow, suction force, and overall length. In some cases, the total axial length of the evacuation tube 1505 extending from a distal end of a ureteroscope can be less than 4 mm. In some cases, the total length can exceed about 4 mm. In such embodiments, an operator can translate the evacuation tube 1505 relative to the ureteroscope to control the distance the evacuation tube 1505 extends from the ureteroscope.

As further shown in FIG. 17B, the liquid supply lumen 1520 can be positioned at a radial end of the evacuation tube 1505. Disposing the liquid supply lumen 1520 at a radial end of the evacuation tube 1505 can provide a large volume in the center of the evacuation tube 1505 to be used as an evacuation lumen 1535 and/or to provide an ablation instrument. Accordingly, the evacuation lumen 1535 can aspirate larger biological objects.

FIGS. 17D-17E illustrate an example of the evacuation tube 1505 further including a transparent sheath 1506. The transparent sheath 1506 can be transparent at visible and laser wavelengths. Accordingly, visible light and laser light can pass through the transparent sheath 1506 undeflected. In such embodiments, the transparent sheath 1506 can protect adjacent structures from heat dissipating from an ablation instrument because energy interacting with the transparent sheath 1506 is not reflected and/or deflected. Additionally, in such embodiments, the transparent sheath 1506 can enhance visibility by providing an optically clear device that is configured to not obstruct visibility for the endoscope. In some cases, the transparent sheath 1506 can enhance engagement with biological objects.

The transparent sheath 1506 can include a distal transparent sheath 1506A and a proximal transparent sheath 1506B. The distal transparent sheath 1506A can axially overlap with the evacuation tube 1505. In some cases, the inner diameter of the distal transparent sheath 1506A overlapping with the evacuation tube 1505 can be greater than an outer diameter of the evacuation tube 1505. In some cases, the distal transparent sheath 1506A can extend distally by an axial distance X0 from the distal end of the evacuation tube 1505. The axial distance X0 can extend between a distal end of the distal transparent sheath 1506A and the distal end of the evacuation tube 1505. In some cases, the distal transparent sheath 1506A can have a step-down in diameter. In some cases, the step-down can be at the distal end of the evacuation tube 1505. The step-down can provide a constant inner diameter for the distal end of the distal transparent sheath 1506A and the distal end of the evacuation tube 1505.

The proximal transparent sheath 1506B can axially overlap with the evacuation tube 1505. In some cases, the inner diameter of the proximal transparent sheath 1506B overlapping with the evacuation tube 1505 can be greater than an outer diameter of the evacuation tube 1505. In some cases, the proximal transparent sheath 1506B can extend proximally by an axial distance X4 from the proximal end of the evacuation tube 1505. The axial distance X4 can extend between a proximal end of the proximal transparent sheath 1506B and the distal end of the evacuation tube 1505.

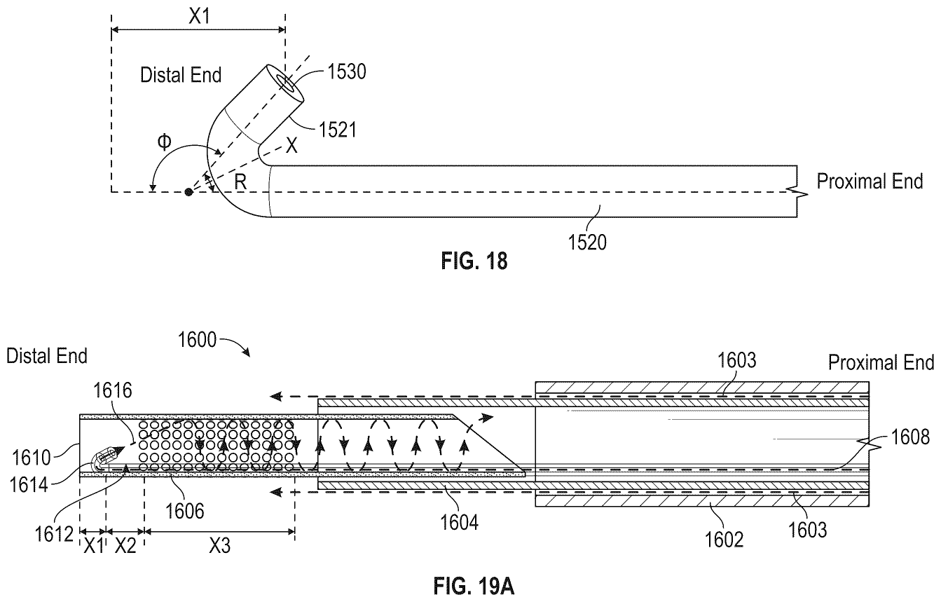

FIG. 17F illustrates a front view of the evacuation tube 1505. As shown in FIG. 17F, the J-tube bend 1521 of the liquid supply lumen 1520 is pivoted about its longitudinal axis relative to a vertical centerline A-A of the evacuation tube 1505 by an angle θ. In some cases, the range of permissible angles θ can depend on the radius of the J-tube bend 1521 as shown in FIG. 18. In some examples, the angle θ can be about 50 degrees when the J-tube bend 1521 is rotated by an angle φ between about 130 and 135 degrees. Pivoting the J-tube bend 1521 at an angle of about 50 degrees can provide a spiraling vortex 1522 of liquid jet through the evacuation tube 1505. In another example, the angle θ can be selected in combination with the angle φ described herein with respect to FIG. 18, the bend radius, and a length of the liquid supply lumen 1520 extending distally from the J-tube bend 1521 so that the outlet of the liquid supply lumen 1520 lies in tangential contact with the inner surface of the evacuation tube 1505. The spiraling vortex 1522 induces a lower pressure at its core (angular momentum is conserved and flow rotates faster near the axis of rotation, or core, which according to Bernoulli's principle reduces the dynamic pressure) and can enhance the Venturi-effect at the distal end. Additionally, the spiraling vortex 1522 can decrease the velocity of the liquid jet before reaching the plurality of vent port openings 1515. In such cases, the axial distance X1 can be decreased since the velocity is decreased by the spiraling vortex 1522. Accordingly, the pressure at the distal end of the evacuation tube 1505 can be reduced further thereby increasing the suction, which can facilitate “bringing” biological objects toward the evacuation tube 1505 and their removal. Additionally or alternatively, the spiraling vortex 1522 can aspirate a biological object about a helical path along the periphery of the evacuation tube 1505. Furthermore, the spiraling vortex 1522 can aspirate the biological objects along a helical path such that an ablation source can contact multiple sides of the biological objects. Accordingly, the biological objects may not interfere with an aspiration device positioned coaxially within the evacuation tube 1505.

FIG. 18 illustrates the J-tube bend 1521 of the liquid supply lumen 1520 described herein. As shown in FIG. 18, the J-tube bend 1521 can be formed by bending the distal end of the liquid supply lumen 1520. In some examples, the J-tube bend 1521 can have an opening 1530 for ejecting the liquid jet from the liquid supply lumen 1520. The J-tube bend 5121 can be rotated relative to the longitudinal axis of the liquid supply lumen 1520 by an angle φ. In some examples, the angle φ can be between about 130 and 135 degrees. For example, the angle φ can be about 135 degrees. In such cases, a longitudinal axis of the second portion can be oriented at an angle relative to a longitudinal axis of first portion such that the liquid is ejected from the liquid supply lumen at an angle relative to the longitudinal axis of the first portion greater than 90 degrees and less than 180 degrees. For example, the liquid can be ejected from the outlet at an angle corresponding to the angle φ.

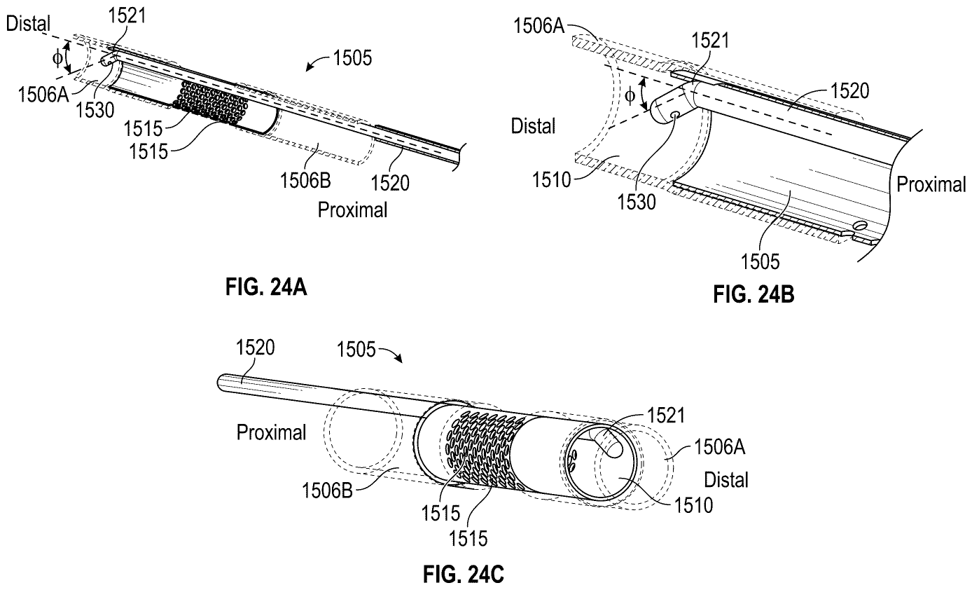

In some cases, the opening 1530 can be positioned along a side wall of the J-tube bend 1521. For example, the opening 1530 can be positioned along a proximally facing side wall of the J-tube bend 1521. In such cases, the J-tube bend 1521 may be configured as a side-firing nozzle, as described herein with respect to FIGS. 24A-24C. The size-firing nozzle can reduce cross-sectional area requirements. In such cases, the angle φ can be less than 90 degrees. For example, the angle φ can be about 45 degrees. In such embodiments, the exit velocity of liquid jet from the J-tube bend can be substantially the same as the example shown in FIG. 18 having an angle φ of 135 degrees and an angle θ of 50 degrees.

In some examples, the J-tube bend 1521 can include a nozzle for outputting a liquid-jet. The nozzle can have an opening size configured to increase the fluid flow from the J-tube bend 1521. In some instances, the opening size of the nozzle (or constriction through the nozzle) can be between 100 microns and 150 microns. The opening of the nozzle can have any shape. For example, the opening of the nozzle may be circular. In some examples, the opening of the nozzle may be an oval or an ellipsis.

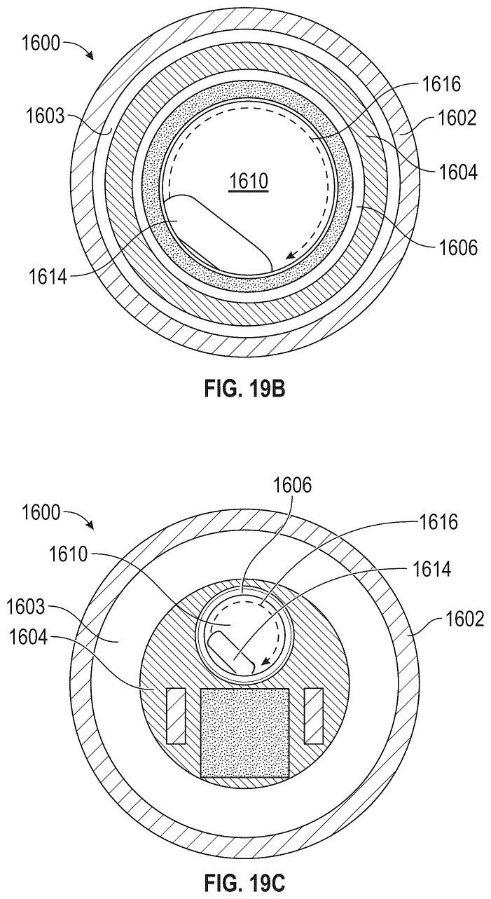

FIGS. 19A-19B respectively illustrate a side view and a front view of a distal end of a system 1600 for removing solid deposits from a subject's urinary system. The system 1600 can be similar to any of the systems described herein except for the differences described herein. The system 1600 can include a plurality of radially nested lumens. In some examples, the system 1600 can include an access sheath 1602, a ureteroscope 1604, and an evacuation tube 1606 (which can be similar to the evacuation tube 1505 or any other evacuation tube described herein). In some examples, the system 1600 can further include an ablation instrument.

The access sheath 1602 can be an access catheter configured to guide the ureteroscope 1604 and/or the evacuation tube 1606 through a urinary system of a subject. For example, the access sheath 1602 can be a ureteral access catheter. In some examples, the access sheath 1602 can be in fluid communication with a first fluid source. The first fluid source may be configured to irrigate the urinary system of the patient. For example, the first fluid source may introduce a first fluid flow 1603 to the urinary system of the patient to expand the organs.

The ureteroscope 1604 can be any ureteroscope (or catheter) described herein. In some examples, the ureteroscope can be inserted through the urethra to diagnose and treat urinary tract problems (such as, kidney stones). In some examples, fluid can be aspirated through the ureteroscope 1604.

The evacuation tube 1606 can be any evacuation tube described herein. The evacuation tube 1606 can include a liquid supply lumen 1608 and an aspiration port opening 1610. In some examples, the evacuation tube 1606 can be configured to provide space for an ablation instrument. For example, an ablation instrument can be configured to operate along the longitudinal axis of the evacuation tube 1606 for an axial approach toward a solid deposit. In such examples, the liquid supply lumen 1608 can be disposed along a side wall of the evacuation tube 1606 and the aspiration port opening 1610 can be positioned at the distal end of the evacuation tube 1606. Providing the liquid supply lumen 1608 along the wall of the evacuation tube 1606 can leave the center of the evacuation tube 1606 unobstructed for the ablation instrument.

The liquid supply lumen 1608 can be a conduit in fluid communication with a second fluid source. For example, the liquid supply lumen 1608 can be in fluid communication with the second fluid source at its proximal end. Accordingly, the liquid supply lumen 1608 can be configured to receive a second fluid flow 1612. The second fluid flow 1612 can have a generally distal flow wherein the fluid flows from the second fluid source at the proximal end to an opening at the distal end.

The liquid supply lumen 1608 can be configured to redirect the fluid flow 1612. In some examples, the liquid supply lumen 1608 can redirect the second fluid flow 1612 at least partially in the proximal direction at the distal end. For example, the liquid supply lumen 1608 can include a J-tube bend 1614 at its distal end. As described herein, the J-tube bend 1614 can have a bend at the distal end relative to the longitudinal axis of the liquid supply lumen 1608.

The liquid supply lumen 1608 can be configured to output a liquid-jet 1616 of the fluid flow 1612. In some examples, the distal end of the liquid supply lumen 1608 can include a nozzle (or a constriction). For example, the nozzle may constrict the flow of liquid thereby increasing the velocity of the fluid flow through the opening resulting in the liquid-jet 1616. The liquid-jet 1616 can form a low-pressure zone at the distal end of the evacuation tube 1606 due to the Venturi-effect. The liquid-jet 1616 can be further redirected by the side walls of the evacuation tube 1606. In some examples, the liquid-jet 1616 can follow a helical flow path along the evacuation tube 1606, which can create a vortex as described herein. The helical flow path can displace a greater volume of liquid than a linear flow path. Accordingly, the helical flow path can further reduce the pressure at the distal end of the evacuation tube 1606 thereby increasing the suction at the distal end, which can facilitate “bringing” or attracting biological objects toward the evacuation tube 1606 and their removal. Additionally, the helical flow path can transport biological objects around the perimeter of the evacuation tube 1606. The helical flow path can thus avoid interfering with the ablation instrument.

As described herein, the evacuation tube 1606 can be open to the surrounding environment at the distal end of the evacuation tube 1606. In such examples, the aspiration port opening 1610 can be positioned at the distal end of the evacuation tube 1606. Accordingly, the evacuation tube 1606 can be used to axially approach and aspirate a solid deposit from the urinary system. For example, the evacuation tube 1606 may axially aspirate solid deposits due to a Venturi-effect caused by the low-pressure zone resulting from the proximally flowing liquid-jet from the J-tube bend 1614 of the liquid supply lumen 1608.

FIG. 19C illustrates a front view of the distal end of the system 1600 for removing solid deposits from a subject's urinary system. FIG. 19C is substantially similar to FIG. 19B. The differences shown in FIG. 19C include a non-concentric alignment and that the ureteroscope 1604 includes additional working elements. The additional working elements can include an optical element, such as, an eyepiece or a camera. As further shown in FIG. 19C, the irrigation lumen can be increased.

FIG. 20 shows a schematic of a ureteroscope system 1690, which can be similar to any of the systems described herein, such as the system 1300, except for the differences described herein. As described herein, the system 1690 can include the access sheath 1602, the ureteroscope 1604, and the evacuation tube 1606. As shown in FIG. 20, the access sheath 1602, the ureteroscope 1604, and the evacuation tube 1606 can be radially nested. For example, the evacuation tube 1606 can be at least partially disposed within a lumen of the ureteroscope 1604 and the ureteroscope can be at least partially disposed within a lumen of the access sheath 1602.

The access sheath 1602 can be in fluid communication with a first fluid source 1601. The first fluid source 1601 can contain a medical fluid. In some examples, the first fluid source 1601 can be a saline bag or container or a container with another IV fluid. Accordingly, the access sheath 1602 can be configured to introduce saline to the urinary system via a first fluid flow 1603 between the access sheath 1602 and the ureteroscope 1604. In some examples, the access sheath 1602 can be integrated with the ureteroscope 1604. For example, the ureteroscope 1604 may include an irrigation channel for accommodating the first fluid flow 1603. The first fluid source 1601 may be positioned at a vertical distance from the ureteroscope 1604.

The ureteroscope 1604 can include on or more lumens. Each of the lumens of the ureteroscope 1604 can include an inlet and an outlet. The one or more lumens can be configured to introduce energy 1650 emitted from an ablation instrument. For example, one of the one or more lumens can receive a laser fiber for emitting a laser at the distal end of the ureteroscope for breaking up and/or fracturing a solid deposit. In some examples, one of the one or more lumens can be configured to receive a liquid supply lumen 1608.

The liquid supply lumen 1608 can be in fluid communication with a second fluid source 1607. In some examples, the second fluid source 1607 can be a saline bag or container or a container with another IV fluid. Accordingly, the liquid supply lumen 1608 can be configured to introduce saline to the system 1690 via a second fluid flow 1609 via a working channel within the ureteroscope 1604. The second fluid source 1607 can be in fluid communication with a high-pressure fluidic pump 1655. Accordingly, the pressure of the second fluid flow 1609 can depend on the operation of the high-pressure fluidic pump 1655. In some examples, the high-pressure fluidic pump 1655 can provide a liquid-jet of saline through the liquid supply lumen 1608.

The ureteroscope 1604 can further include a return lumen 1605. In some examples, the return lumen 1605 can be within a working channel of the ureteroscope 1604 occupied by the supply lumen and/or an ablation instrument. The return lumen 1605 can be configured to receive fluid and/or solid deposits from the urinary system. For example, the return lumen 1605 can receive an aspiration flow 1664. In some examples, the return lumen 1605 can be in fluid communication with a container 1660. The container 1660 can be configured to receive waste. For example, the container 1660 can be configured to receive saline, solid deposits, and/or other biological fluids. In some examples, the return lumen 1605 can be in fluid communication with an aspiration pump 1665. The aspiration pump 1665 may assist in aspirating fluid along the aspiration flow 1664.

The ureteroscope 1604 can further include a seal 1622. The seal 1622 can be configured to fluidly seal at least part of the ureteroscope 1604. In some examples, the seal 1622 can be disposed at the proximal end of the ureteroscope 1604. For example, the seal 1622 can extend around the one or more lumens to prevent the first fluid flow 1603 from flowing proximally out of the access sheath 1602. Accordingly, the pressure within the urinary system can be controlled by preventing pressure loss through leaks at the proximal end of the access sheath 1602. Thus, the first fluid flow 1603 can be limited to outputting fluid into the urinary system.

The evacuation tube 1606 can be the same or similar to the evacuation tubes described herein. For example, the evacuation tube 1606 can be positioned at the distal end of the liquid supply lumen 1608. The evacuation tube 1606 can further include one or more aspiration port openings 1610 and one or more vent port openings 1615. In some examples, the one or more aspiration port openings 1610 can be a single aspiration port opening at the distal end of the evacuation tube 1606. The one or more vent port openings 1615 can be disposed around the annular surface of the evacuation tube 1606. In some examples, the liquid supply lumen 1608 can include the J-tube bend 1614 at the distal end of the evacuation tube 1606 for redirecting the second fluid flow 1609. As further shown in FIG. 20, the energy 1650 from the ablation instrument can pass through the center of the evacuation tube 1606.

The system 1690 can be introduced to a portion of a subject's urinary system. The first fluid flow 1603 can maintain or expand the volume of the organs of the urinary system to enhance visibility and navigation of the system 1690 through the urinary system. The high-pressure fluidic pump 1655 can be activated to provide a high flow rate of fluid flow through the liquid supply lumen 1608 to the distal end of the evacuation tube 1606. The second fluid flow 1609 can provide a low-pressure zone at the distal end of the evacuation tube 1606 via the J-tube bend redirecting the liquid-jet into the evacuation tube 1606. The redirection of fluid can induce a Venturi effect, create a jet pump, and/or create a spiraling vortex flow to maintain consistent level of low pressure at the distal end of the evacuation tube 1606 for aspirating solid deposits located in the subject's kidney. Accordingly, fluids and solid deposits within the urinary system positioned distally of the evacuation tube 1606 can be aspirated into the return lumen 1605 of the ureteroscope. The energy 1650 of the ablation instrument can be used to break up and fracture solid deposits that are too large to pass through the return lumen 1605. The aspiration pump 1665 can be activated to provide aspiration through the return lumen 1605.

The system 1690 can maintain safe levels of temperature during the surgical procedure. As described herein, prolonged use of an ablation device that increases the temperature of the fluid environment above 43 degrees Celsius may cause tissue damage and protein denaturation. The system 1690 can maintain a safe operating temperature (such as, below 43 degrees Celsius) due to one or more of material selection, insulation, and fluid balancing. In some examples, the evacuation tube 1606 can be formed, at least partially, from a metal, such as stainless steel. The liquid supply lumen 1608 can be insulated from the warmer fluid being aspirated out through an evacuation lumen and/or the return lumen 1605. The insulation can have a low thermal conductivity (such as, 40-80 times lower than stainless steel) and may be produced in thin dimensions (such as, 100 microns or less) so as to maximize the available volume for fluid flow. Additionally, the insulation may be biocompatible. For example, the insulation may be polyimide, polyetheretherketone (PEEK), fluoropolymers, and/or silicone. This design can prevent heat transfer between the warmer fluid being aspirated from the urinary system and the fluid being introduced into the urinary system.

In some cases, the fluid can be introduced into the urinary system at a high flow rate (such as, 40-100 mL/min). Such high flow rate can cause a faster exchange of the fluid warmed in the urinary system as a result of being heated by the ablation instrument with cooler (such as, room-temperature) fluid delivered from one or more of the first fluid source 1601 or the second fluid source 1607, which can assist with temperature management. Accordingly, the system 1690 can effectively control the temperature within the urinary system and prevent temperatures from rising above a threshold associated with safety (such as, 43 degrees Celsius). In some cases, the ablation instrument can be configured to operate at or below a threshold power level (such as, 20 Watts) when emitting energy 1650 to minimize the heat added to the urinary system.

Fluid balance can be maintained by introducing fluid into the urinary system at about the same rate as removing aspirating fluid via the evacuation tube. For example, the combined rate of fluid flow from the first fluid source 1601 and the second fluid source 1607 can be about the same as the rate of fluid flow provided by the aspiration pump 1665.

In some cases, at least a portion of the evacuation tube 1606 (and/or catheter) can be formed from fluoropolymers and other plastics (such as, PTFE, fluoroelastomers, or the like) or any combination of fluoropolymers and other plastics and such materials. For example, the distal tip of the evacuation tube 1606 (and/or catheter) can be formed from fluoropolymers and other plastics. Fluoropolymers and other plastics provide optical clarity at visible wavelengths, allowing visualization of the laser fiber position through the evacuation tube and high transmission of light at the wavelengths emitted by the ablation instrument (such as, a laser). As a result, the material would not absorb energy emitted by the ablation instrument, thereby avoiding or limiting the possible damage to the evacuation tube 1606. The evacuation tube 1606 may be formed from material that absorbs the energy (such as, stainless steel) and forming a portion of the evacuation tube 1606 from fluoropolymers and other plastics can promote safety and reliability.

In some cases, the evacuation tube 1606 can be formed at least partially from a plastic material (such as, a thermoplastic material). For example, a proximal end of the evacuation tube 1606 can be formed of a plastic material. In some cases, the plastic material can be flexible for feeding the evacuation tube through a patient's anatomical structure and sufficiently strong to withstand the internal pressures from the liquid.

The system 1690 can also provide pressure controls via the access sheath 1602. The access sheath 1602 can include a seal 1620. The seal 1620 can be configured to fluidly seal at least part of the access sheath 1602. In some examples, the seal 1620 can be an annular structure with an opening extending therethrough. The seal 1620 can be configured to engage the exterior surface of the ureteroscope 1604. For example, the seal 1620 may be configured to engage a ureteroscope 1604 having an outer dimension between 6 French and 9 French. In some examples, the seal 1620 can be disposed at the proximal end of the access sheath 1602. For example, the seal 1620 can extend around the ureteroscope 1604 to prevent the first fluid flow 1603 from flowing proximally out of the access sheath 1602. Accordingly, the pressure within the urinary system can be controlled by preventing pressure loss through leaks at the proximal end of the access sheath 1602. Thus, the first fluid flow 1603 can be limited to introducing fluid into the urinary system. Existing access sheaths do not form a seal around the ureteroscope, but instead provide a natural leak path thereby failing to provide precise and consistent pressure control.

The first fluid source 1601 can be positioned at one or more heights to provide a requisite pressure head for a desired flow rate through the access sheath 1602 (such as, to maintain fluid balancing as described herein). The pressure of the first fluid flow 1603 can depend on the height of the first fluid source 1601, the density of the fluid within the first fluid source 1601, and the acceleration due to gravity. For example, the pressure of the first fluid flow 1603 can follow the relationship P=hρg, wherein h is the height of the first fluid source 1601 relative to the access sheath 1602, ρ is the density of the fluid (such as, 2160 kg/m³ for saline), and g is the acceleration due to gravity (such as, 9.81 m/s²). Accordingly, the pressure can be proportional to the height of the first fluid source 1601. For example, the pressure can increase as the height of the first fluid source 1601 increases. In some implementations, a sensor 1654 can be in fluid communication with the first fluid flow 1603 to monitor the flow rate of the first fluid flow. Provision of liquid from the first fluid source 1601 can facilitate fluid balancing and assist with clearing any obstructions or blockages in the system 1690. Additionally, the provision of liquid from the first fluid source 1601 can reduce temperatures and enhance visibility.

In some cases, instead of adjusting the vertical height of the first fluid source 1601 to regulate the irrigation flow into the anatomical structures, one or more valves may be implemented for regulating the irrigation flow into the anatomical structure. The valves may be configured to provide pressure relief to the anatomical structure. For example, any of the one or more valves may be a check valve. In some examples, the irrigation lumen of the sheath 1602 may be a bi-directional lumen configured to accommodate fluid distally from the first fluid source 1601 to the anatomical structure and proximally from the anatomical structure to the first fluid source 1601. Accordingly, irrigation fluid may flow distally from the first fluid source 1601 to the anatomical structure when the pressure head between the first fluid source 1601 and the anatomical structure is positive. Similarly, the irrigation fluid may flow proximally from the anatomical structure to the first fluid source 1601 when the pressure head between the first fluid source 1601 and the anatomical structure is negative. Thus, the sheath 1602 and the first fluid source 1601 can mitigate overpressure within the anatomical structure by providing an outflow from the anatomical structure to the first fluid source 1601. In some examples, overpressure in the anatomical structure can result in infection and sepsis. For example, pressurizing anatomical structures may force the irrigation fluid along with bacteria or other foreign objects into or through the walls of the anatomical structure. The corresponding pressure for overpressurization may be about 30 mmHg. Accordingly, when the pressure within the anatomical structure reaches 30 mmHg, one or more check valves may allow backflow into the first fluid source 1601. For example, reaching or exceeding the cracking pressure within the anatomical structure may instantly activate the one or more check valves which may remain open until the pressure within the anatomical structure drops below the cracking pressure threshold. In some examples, the cracking pressure for the one or more check valves may exceed 30 mmHg over a duration of time. For example, the duration of time may be about 5 seconds. Accordingly, the internal pressure within the anatomical structure may exceed 30 mmHg for a short duration until the cracking pressure is reached to initiate backflow into the first fluid source 1601. Pressure regulation may be passive or controlled. For example, the one or more check valves may have innate cracking pressures to passively regulate the pressure. Alternatively, one or more sensors and control valves may be used with a control system to actively regulate irrigation flow.

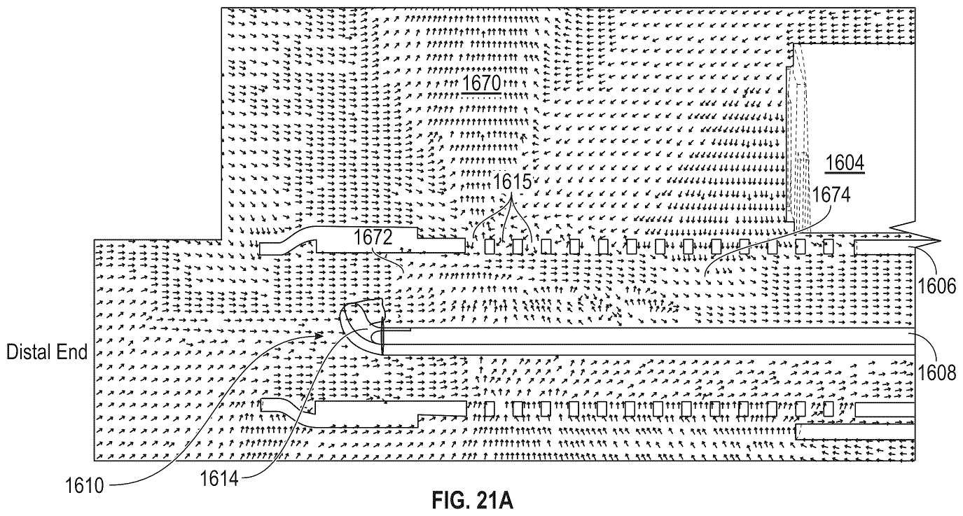

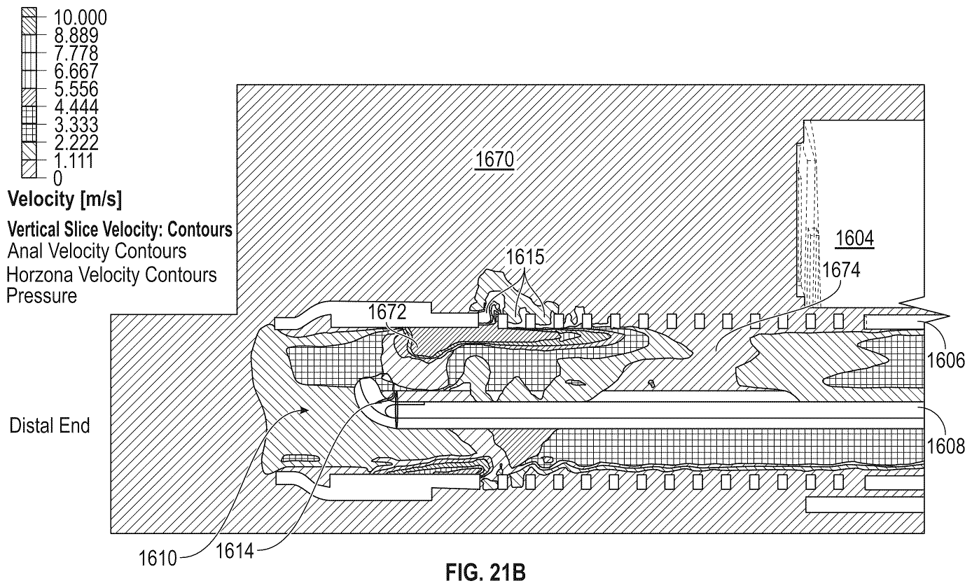

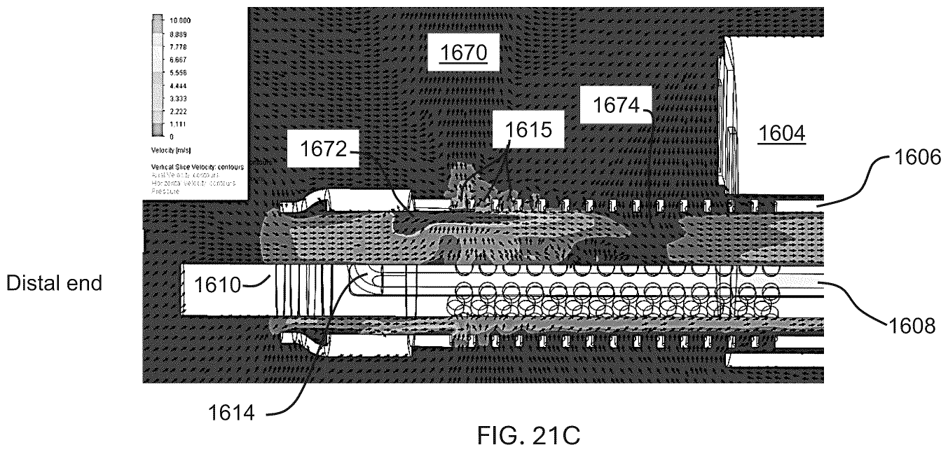

FIGS. 21A-21C illustrate a pressure field and fluid graph within the system 1690. As shown in FIGS. 21A-21C, the system 1690 can include the evacuation tube 1606 described herein. The evacuation tube 1606 can include an aspiration port opening 1610, a liquid supply lumen 1608 having a J-tube bend 1614 at the distal end of the evacuation tube 1606, and a plurality of vent port openings 1615.

As shown in FIG. 21A, the fluid flow is shown to enter the aspiration port opening 1610 at the distal end of the evacuation tube 1606 and recirculate through the one or more vent port openings 1615. As further shown in FIG. 21A, fluid can also be aspirated through the one or more vent port openings 1615. Aspirating fluid through the one or more vent port openings 1615, as shown in FIG. 21A can enhance visibility by aspirating debris and fragments through additional openings.

As shown in FIG. 21B, the liquid-jet from the liquid supply lumen 1608 can increase the velocity of the fluid within the evacuation tube 1606. For example, a peak velocity can be positioned along the boundary of the evacuation tube 1606 in the direct flow path of the liquid-jet from the liquid supply lumen 1608. The high velocity of the liquid-jet can be exhaust at least some of the liquid-jet fluid through one or more of the vent port openings 1615. The peak velocity can correspond to a minimum pressure due to the Venturi effect wherein fluid pressure reduces when fluid velocity increases. To balance pressures, the surrounding liquid 1670 can enter the distal end of the evacuation tube 1606 via the aspiration port opening 1610. The surrounding liquid 1670 may be prevented from balancing the pressures via one or more of the vent port openings 1615 because the high-velocity liquid-jet fluid may be exiting through the vent port openings 1615. However, the surrounding liquid 1670 may enter the evacuation tube 1606 via one or more vent port openings 1615 located downstream of the J-tube bend 1614. Accordingly, the surrounding liquid 1670 and corresponding solid deposits and/or debris may be configured to enter the evacuation tube 1606 via the aspiration port opening 1610 and one or more of the downstream vent port openings 1615. This may enhance visibility for an operator by aspirating additional debris through the vent port openings 1615.

The devices and methods described herein can be compatible with existing laser systems and can facilitate optimal laser fiber positioning, fluid management, and suction control. For example, the system's 1690 suction and fluid balancing features can improve kidney stone positioning during ablation and rapid clearing of the dust field for improved visibility. As another example, the system 1690 can provide fluid balancing, which advantageously reduces safety risks by maintaining lower intrarenal pressures, enabling continuous fluid circulation for low temperatures, and continuously removing dust. In some examples, the approaches described herein can be compatible with existing ureteroscopes or catheters. Approaches described herein can enable faster, more efficient surgeries thereby reducing the time in which complications can occur, reduce operator burden (such as, fatigue), and reduce overall healthcare costs.

FIG. 21C illustrates a color illustration of FIGS. 21A-21B.

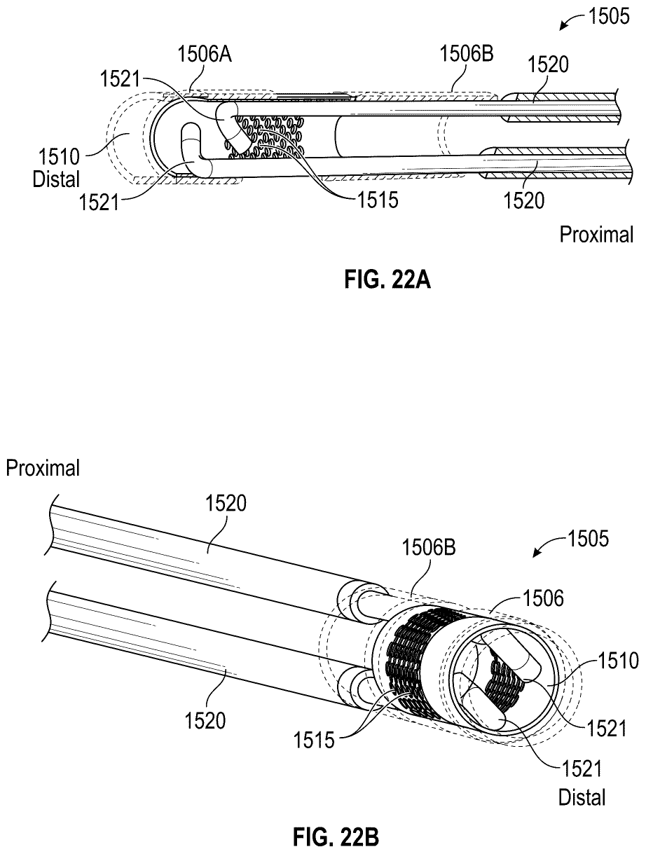

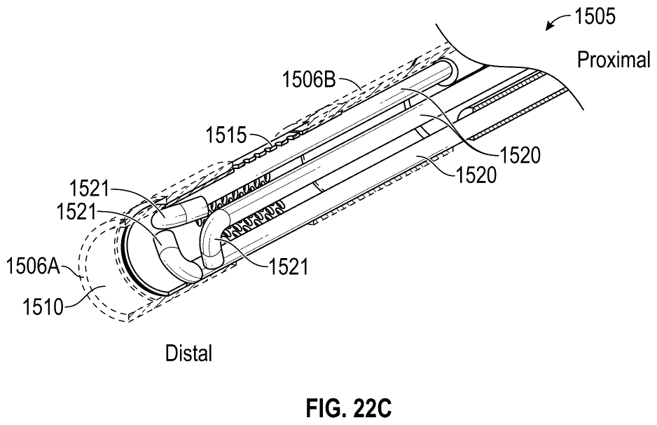

FIGS. 22A-22C illustrate examples of an evacuation tube 1505 including a plurality of liquid supply lumens 1520. As shown in FIGS. 22A-22C, the evacuation tube 1505 can be the same or similar to the evacuation tube 1505 described herein with respect to FIGS. 17A-17F. Each of the plurality of liquid supply lumens 1520 can be the same or similar to the liquid supply lumen 1520 described above with respect to FIGS. 17A-17F. For example, each of the plurality of liquid supply lumens 1520 can include a J-tube bend 1521 configured to redirect a fluid flow from a distal flow direction to a proximal flow direction. In some examples, each of the plurality of liquid supply lumens 1520 can be configured to output a liquid jet configured to form a helical flow. Accordingly, the plurality of liquid supply lumens 1520 can be configured to eject the liquid outside the respective liquid supply lumen from an orifice. In such cases, the liquid can be ejected from a plurality of outlets. The plurality of outlets can be orifices and/or nozzle openings. Ejecting the liquid from a plurality of outlets can increase the velocity of the fluid flow within the evacuation lumen.

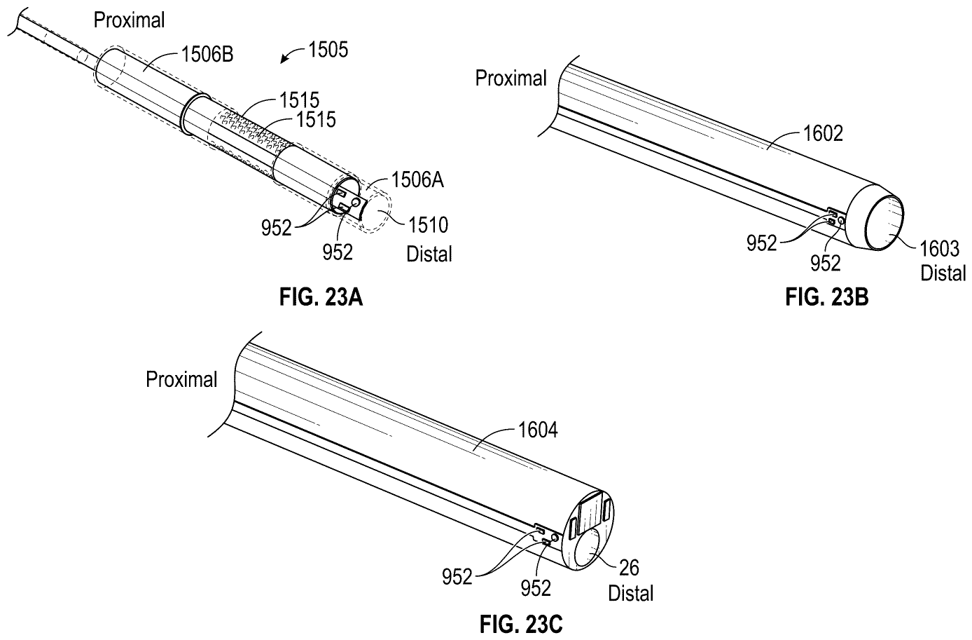

FIGS. 23A-23C illustrate examples of one or more sensors 952 positioned at a distal end of an evacuation tube 1505, a sheath 1602, and/or a ureteroscope 1604. As described herein, the one or more sensors 952 can be configured at a distal end of the system and configured to measure a temperature and/or pressure at the distal end of the system. The one or more sensors 952 can be the same or similar to the one or more sensors 952 described herein with respect to FIG. 11.

FIGS. 24A-24C illustrate examples of an evacuation tube 1505 including a liquid supply lumen 1520 with an opening on a sidewall of the J-tube bend 1521 for side-firing. As described herein with respect to FIG. 18, the opening 1530 can be positioned along a side wall of the J-tube bend 1521. For example, the opening 1530 can be positioned along a proximally facing side wall of the J-tube bend 1521. In such cases, the J-tube bend 1521 may be configured as a side-firing nozzle. In some cases, the side-firing nozzle can be configured to eject the liquid at an angle between 90 and 180 degrees relative to the longitudinal axis of the liquid supply lumen 1520. In such cases, a longitudinal axis of the second portion can be oriented at an angle relative to a longitudinal axis of first portion such that the liquid is ejected from the liquid supply lumen at an angle relative to the longitudinal axis of the first portion greater than 90 degrees and less than 180 degrees. In some cases, the liquid ejection angle relative to the longitudinal axis of the liquid supply lumen 1520 can depend on the angle φ. For example, the liquid ejection angle can be perpendicular to the angle φ.

The side-firing nozzle can reduce cross-sectional area requirements. In such cases, the angle φ can be less than 90 degrees. For example, the angle φ can be about 45 degrees. In such examples, the exit velocity of liquid jet from the J-tube bend can be substantially the same as the example shown in FIG. 18 having an angle φ of 135 degrees and an angle θ of 50 degrees. Additionally, the liquid supply lumen 1520 can be rotated about the longitudinal axis by an angle θ as described above with respect to FIG. 17F. The closer the angle φ is to 90 degrees, the shallower the angle θ. By comparison, the close the angle φ is to 90 and/or 180 degrees, the greater the angle θ.

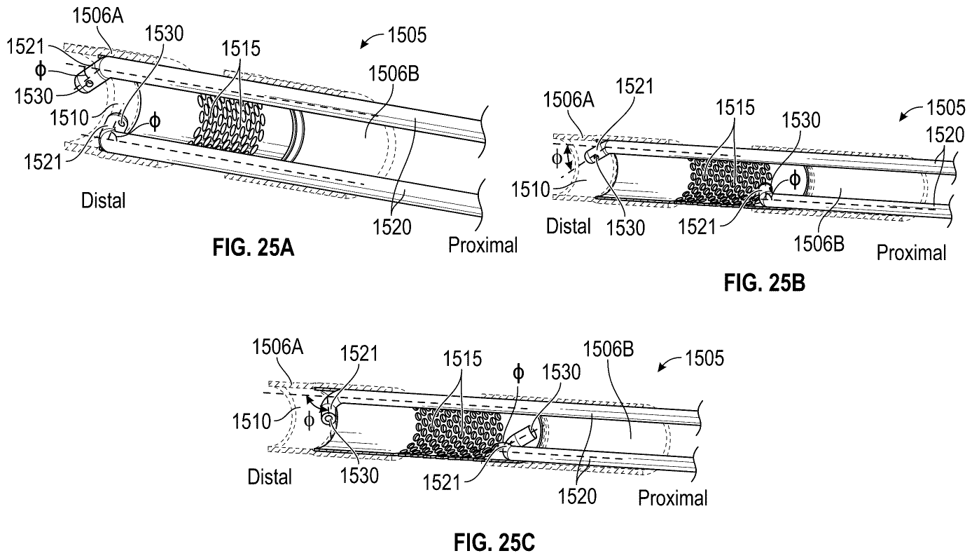

FIGS. 25A-25C illustrate examples of an evacuation tube 1505 including a plurality of axially displaced liquid supply lumens 1502. As shown in FIGS. 25A-25C, the evacuation tube 1505 can include one or more liquid supply lumens 1520 with an output at the distal end of the evacuation tube 1505 and one or more liquid supply lumens 1520 with an output on the other side of the vent port openings 1515. Accordingly, multiple jet-pump effects can be created intermittently through the evacuation tube 1505. In some examples, the liquid jets emitted from the multiple supply lumens and/or fluid flows may intersect.

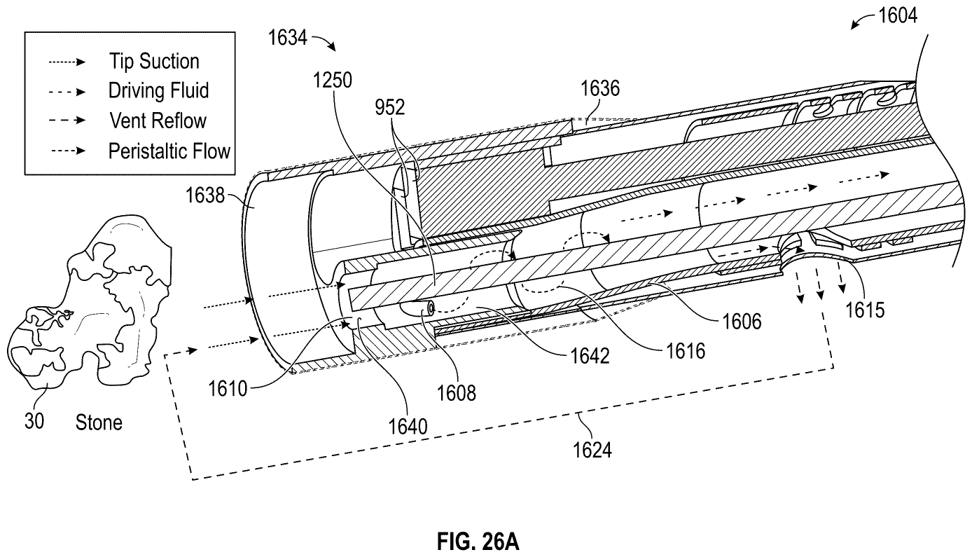

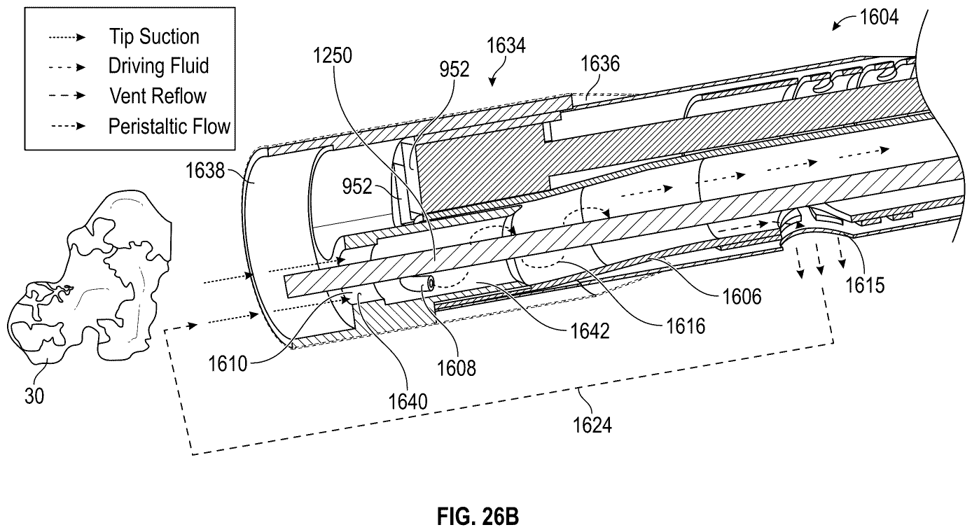

FIGS. 26A-26B illustrates an example of a distal end of a ureteroscope 1604. The ureteroscope 1604 may be the same or similar to the ureteroscopes described herein except for the differences described below.

The ureteroscope 1604 can include a distal tip 1634. The distal tip 1634 can be provided at the distal end of the ureteroscope 1604. The distal tip 1634 can be a working end of the ureteroscope 1604. The distal tip 1634 can be integrated and/or an add-on device. For example, the distal tip 1634 can be built into the ureteroscope 1604. Alternatively, the distal tip 1634 can be added as an after-market modification to an existing ureteroscope. In such examples, the distal tip 1634 can be a two-piece variation that can be secured to the distal end of the ureteroscope 1604 via a securing device 1636. In some examples, the securing device 1636 can be a heat-shrink tubing. In some examples, the distal tip 1634 can be formed of a high-performance composite material. In some examples, the distal tip 1634 can be formed of a reinforced fluoropolymer (PFA) with ceramic nanoparticles. The PFA with ceramic nanoparticles can provide exceptional laser energy resistance and mechanical durability. In some examples, the distal tip 1634 can be formed of jewels. For example, the distal tip 1634 can include sapphire or synthetic diamond inserts. The jewels can enhance laser resistance, provide optical clarity, and increase the longevity of the distal tip 1634. In some examples, the distal tip 1634 can be formed of an optically clear material. For example, the distal tip 1634 can be formed of a polymer (e.g., polycarbonate).

The distal tip 1634 can include the aspiration port opening 1610. The aspiration port opening 1610 can be configured to receive the biological objects 30. In some examples, a low pressure may be present at the distal tip 1634. The low pressure can attract biological objects 30 through the aspiration port opening 1610 into an inner lumen of the ureteroscope 1604 (e.g., the evacuation tube 1606). For example, a liquid jet can be proximally discharged from the supply lumen 1608 to create the low-pressure within the ureteroscope 1604 via a Venturi and/or Entrainment effect

The distal tip can further include the cup 1638, a choke 1640 and a working channel extension 1642.

The cup 1638 can include an annular wall extending distally away from the distal end of the ureteroscope 1604. In some examples, the cup 1638 can be formed of an optically clear material. For example, the cup 1638 can be formed of a polymer (e.g., polycarbonate). The length of the cup 1638 can be optimized to grasp biological objects 30 while not obstructing the field of view of the optical sensor. Grasping biological objects 30 can be improved by increasing the length of the cup 1638. For example, the cup 1638 may accumulate and retain fragments of the biological objects 30. However, increasing the length of the cup 1638 can impede or object the field of view of one or more sensors 952.

The choke 1640 can form a channel into the evacuation tube 1606. The channel of the choke 1640 can be a constricted lumen relative to the evacuation tube 1606. In some examples, the geometry of the choke 1640 can correspond to the geometry of the aspiration port opening 1610. The choke 1640 can be configured to prevent large biological objects 30 from entering the evacuation tube 1606. In some examples, the choke 1640 can be configured to be thermal resistant and laser resistant (such as, have fluoropolymer thermal and laser resistant properties). For example, the choke 1640 can be formed from a jewel (e.g., synthetic sapphire). Accordingly, aspirated biological objects will be sized to fit through the evacuation tube 1606 without blocking and/or plugging the evacuation tube 1606. The length of the choke 1640 can be minimized to reduce clogging while remaining long enough to be robust. A reduced diameter of the choke 1640 can ensure that any stone fragment that passes through are small enough to continue up the working channel and out of the device.

The working channel extension 1642 can extend distally from the distal end of the evacuation tube 1606 of the ureteroscope 1604 to the proximal end of the choke 1640. In some examples, the supply lumen 1608 may output a liquid jet into the working channel extension 1642.

The distal tip 1634 can further include and/or configured to receive one or more sensors 952. In some examples, the one or more sensors 952 can include an optical sensor and/or one or more light sources. The one or more sensors 952 can include a camera. For example, the one or more sensors 952 can include a CMOS camera. In some examples, the one or more sensors 952 can include an LED light source.

As shown in FIGS. 26A-26B, an ablation instrument 1250 may be positioned within the ureteroscope 1604. The ablation instrument 1250 may be configured to move axially within the ureteroscope 1604. In some examples, the ablation instrument 1250 may be moved proximally or at least partially retracted into the ureteroscope 1604. Retracting the ablation instrument 1250 can cause a low-pressure zone thereby inducing biological objects toward the evacuation tube 1606. Accordingly, retracting the ablation instrument 1250 may be used for basketing a biological object 30, multiple biological objects 30, one or more fragments of biological object(s) 30, and/or any combination biological object(s) 30 or fragment(s). In some examples, the ablation instrument 1250 may be oscillated axially to unblock and/or prevent fragmented biological objects 30 from occluding the aspiration port opening 1610 and/or the evacuation tube 1606.

The ureteroscope 1604 described herein at least with reference to FIG. 26A-26B (or any other improved ureteroscopes describe herein) may provide more efficient basketing as compared to existing ureteroscopes. Because existing ureteroscopes do not provide sufficient suction, when basketing, a physician would need to move the ureteroscope relative to the outer sheath and/or axially oscillate the energy source (e.g., ablation instrument) to increase the suction, which is burdensome and time consuming. Further, because existing ureteroscopes do not maintain fluid balance, suction would need to be lowered or paused to avoid collapse of the kidney. In contrast, the more efficient basketing provided by ureteroscope 1604 is attributable to one or more of: high levels of suction provided at the distal end of the ureteroscope 1604, maintenance of fluid balance, and/or omitting axial oscillations of the ablation instrument 1250. In some instances, the ureteroscope 1604 provides pressure of 150-700 mmHg at the distal face of the evacuation tube 1606, while maintaining overall pressure inside the kidney at 0-75 mmHg.

As described herein with reference to FIGS. 6A-6F, 11, 15 and 20, the supply lumen 1608 may be in fluid communication with a liquid source 440, a fluid source 940, 1340, and/or a second fluid source 1607. The supply lumen 1608 may output a high flow rate of liquid (e.g., a liquid jet) into the evacuation tube 1606. The liquid jet may induce a Venturi and/or entrainment effect at the aspiration port opening 1610. For example, the liquid jet may provide a low-pressure zone at the aspiration port opening 1610 thereby attracting biological objects 30 toward the aspiration port opening 1610. In some examples, the liquid jet may output a fluid flow rate of about 10-100 ml/min. The corresponding suction force may be a function of the pressure difference between the distal end of the evacuation tube 1606 and the cross sectional area of the aspiration port opening 1610. In some examples, the corresponding suction force may correspond to a change in pressure across the choke 1640 multiplied by the cross-sectional area of the aspiration port opening 1610. For example, the pressure within the cavity may be between 0 and 75 mmHg (0 and 9.9 kPa), the pressure at the low-pressure zone within the evacuation tube 1606 may be between −150 and −700 mmHg (−19.9 and −93.3 kPa), and the cross-sectional area of the aspiration port opening 1610 may be between 0.25 and 80 mm². Accordingly, the resulting suction force may be between about 4.9 mN and 8.3 (0.0011 and 1.86 lbf). Accordingly, the biological objects 30 may be secured to the cup 1638 while an ablation instrument 1250 breaks up the biological objects 30 into fragments sized to pass through the choke 1640. The suction force generated by the liquid jet from the supply lumen 1608 into the evacuation tube 1606 may contribute to a more efficient basketing by effectively grasping the biological object 30 without mechanical means and/or retracting the ablation instrument 1250.

TABLE 1
Example Resulting Suction Force Values

| Exterior Pressure | | Internal Pressure | Cross-Sectional Area | | Force | |
|---|---|---|---|---|---|---|
| 0 mmHg | (0 Pa) | −150 mmHg (−19.9 kPa) | 0.25 mm² | (.00000025 m²) | 4.9 | mN |
| 75 mmHg | (9.9 kPa) | −150 mmHg (−19.9 kPa) | 0.25 mm² | (.00000025 m²) | 7.5 | mN |
| 0 mmHg | (0 kPa) | −700 mmHg (−93.3 kPa) | 0.25 mm² | (.0000025 m²) | 23.3 | mN |
| 75 mmHg | (9.9 Pa) | −700 mmHg (−93.3 kPa) | 0.25 mm² | (.00000025 m²) | 25.8 | mN |

| | | | | | |
|---|---|---|---|---|---|
| 0 mmHg | (0 Pa) | −150 mmHg (−19.9 kPa) | 80 mm² | (.00008 m²) | 1.5N |
| 0 mmHg | (0 Pa) | −700 mmHg (−93.3 kPa) | 80 mm² | (.00008 m²) | 7.5N |
| 75 mmHg | (9.9 Pa) | −150 mmHg (−19.9 kPa) | 80 mm² | (.00008 m²) | 2.4N |
| 75 mmHg | (13.3 kPa) | −700 mmHg (−93.3 kPa) | 80 mm² | (.00008 m²) | 8.3N |

High flow rates may introduce risks. For example, a high flow rate from the liquid jet (10-100 ml/min) can generate a low-pressure zone creating a suction force into the evacuation tube 1606 as described above. In some examples, the resulting entrainment flow rate can range between 15 and 300 ml/min. Accordingly, the fluid located exterior to the ureteroscope 1604 may be drained as the ureteroscope 1604 operates. As described herein, the ureteroscope 1604 may be used within a cavity of a subject such as a kidney. A kidney may have a volume ranging between 5 and 50 ml. A fluid may be present in the kidney to assist with expanding the kidney to assist with visualization for an operator and/or to assist with maneuvering the ureteroscope 1604 within the kidney. In such examples, a volume of fluid within the kidney may be drained between 19.8 seconds and 3.3 minutes. Draining the fluid from the kidney may deflate the kidney thereby reducing visibility and/or maneuverability. Accordingly, there remains a need to regulate fluid volume within the cavity for continuous use of the ablation instrument.

TABLE 2
Example Drain Times

| | | |
|---|---|---|
| Entrainment Flow Rate | Volume | Drain Time |

| | | | | | |
|---|---|---|---|---|---|
| 15 | ml/min | 5 | ml | 0.33 min. | (19.8 sec.) |
| 15 | ml/min | 50 | ml | 3.33 min. | (198 sec.) |
| 300 | ml/min | 5 | ml | 0.016 min. | (1 sec.) |
| 300 | ml/min | 50 | ml | 0.16 min. | (10 sec.) |

As described herein with reference to at least FIG. 20, fluid may be provided to pressurize the cavity of the subject. Pressurizing the cavity may inflate or expand the cavity to increase the visibility for the operator and maneuverability of the ureteroscope 1604. In some examples, a first fluid source 1601, a sheath 1602, and a seal 1620 as described herein may be provided in fluid communication with the cavity to provide an inlet fluid flow to the cavity to replace fluid being aspirated through the ureteroscope 1604. The seal 1620 may prevent fluid from exiting one end of the sheath 1602 such that the fluid flow may be configured to exit only one end of the sheath 1602. However, this option may decrease the operable size of the ureteroscope 1604 since the ureteroscope 1604 and the sheath 1602 must fit within the ureter of the subject. Accordingly, there remains a need to regulate the fluid volume within the cavity for continuous use of the ablation instrument without an outer sheath.

Additionally, as described herein with reference to FIGS. 2D-2E, and 2G, biological objects 30 may be broken up or fractured by an ablation instrument 1250. In some examples, the ablation instrument 1250 may be a laser fiber. Activating the ablation instrument 1250 may introduce heat into the cavity. For example, activating a laser fiber may introduce heat energy into the cavity. In some examples, the heat energy may result in cavitation of fluid within the cavity. Additionally, the cumulative addition of heat energy into the cavity may increase the temperature of the fluid within the cavity. In some examples, the temperature of the fluid may exceed a safe temperature threshold. Accordingly, the fluid may damage the subject's tissue when the fluid exceeds the safe temperature threshold. In some examples, 43 degrees Celsius may be the safe temperature threshold. One option may be to provide cool-down periods during the procedure to allow the fluid temperature to decrease to a safe operating temperature before activating the ablation instrument 1250. This option may increase the duration of the procedure thereby reducing the efficiency of such procedures. Accordingly, there remains a need to regulate temperatures of the fluid for continuous use of the ablation instrument.

The ureteroscope 1604 can regulate temperature and pressure within the cavity by recirculating fluid flow between the evacuation tube 1606 and the cavity. In some examples, the ureteroscope 1604 does not include a second fluid source or an outer sheath. Instead, the temperature and the pressure may be regulated solely by recirculating the fluid between the ureteroscope and the external environment. For example, the liquid jet may act to entrain fluid from an exterior volume of the ureteroscope 1604 into the evacuation tube 1606. The cumulative fluid flow (including both the liquid jet and entrained fluid) may flow proximally through the evacuation tube 1606. As described herein, the ureteroscope 1604 may include one or more vent port openings 1615 in fluid communication with the evacuation tube 1606 and the external environment. Accordingly, fluid flowing through the evacuation tube 1606 may exit the evacuation tube 1606 into the external environment via the vent port openings 1615. In some examples, heat may be exchanged between the entrained fluid heated by the use of the ablation instrument 1250 and the fluid provided by the liquid jet. Accordingly, the recirculated fluid 1624 may be cooled to a safe temperature such that the cumulative heat within the cavity does not exceed the safe temperature threshold. In some examples, the flow rate of the fluid exiting through the vent port openings 1615 can be the same or similar to the flow rate of the fluid entrained through the aspiration port opening 1610. Thus, the reflow fluid circuit between the external environment, the aspiration port opening 1610, the evacuation tube 1606, and the vent port openings 1615 may decouple the overall cavity fluid exchange rate from the flow rate through the distal tip 1634. Accordingly, the volume of fluid within the cavity may remain constant thereby providing a constant pressure to the cavity without requiring a second fluid source or outer sheath. Accordingly, the ureteroscope 1604 may allow for an extremely high flow rate through the distal tip 1634 without the risks associated with a high fluid exchange rate in the small intrarenal space.

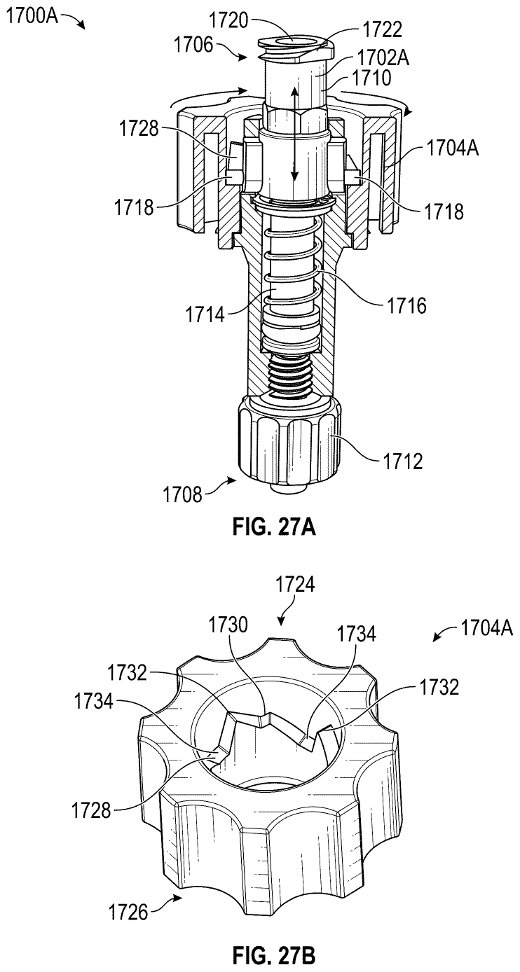

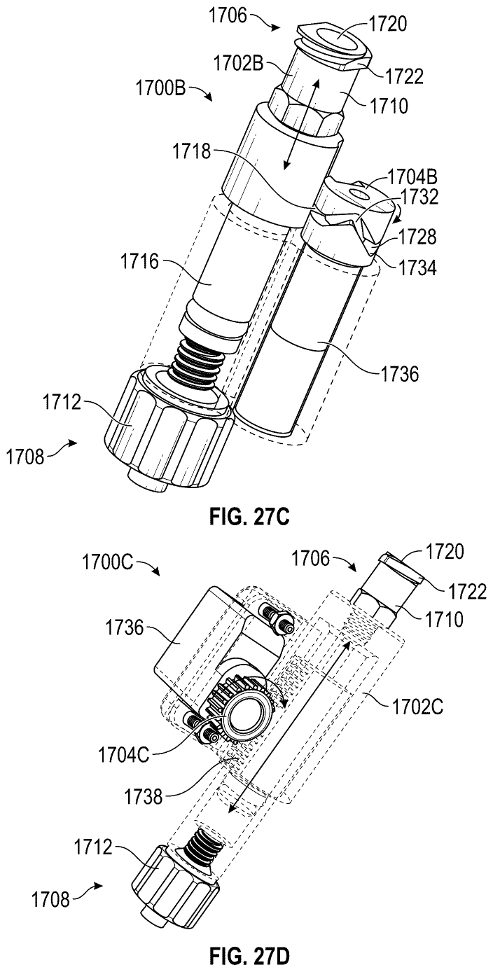

FIGS. 27A-27D illustrate examples of a reciprocator configured to axially move an ablation instrument 1250 disposed within a ureteroscope. In some examples, the ureteroscope may be a manually controlled device. For example, the ureteroscope may be configured to be handled and manipulated by an operator for controlling flexion of the catheter, axial movement of the ablation instrument 1250, and/or oscillating axial movements of the ablation instrument 1250 relative to the ureteroscope. In some examples, the ureteroscope may be positioned within a kit configured to robotically control the ureteroscope. For example, the ureteroscope may be coupled with a reciprocator. The reciprocator may be configured to couple with the ablation instrument 1250 for controlling axial movements of the ablation instrument 1250 relative to the ureteroscope.

FIG. 27A illustrates a first example of a reciprocator 1700A. The reciprocator 1700A can include a body portion 1702A and a rotating cam 1704A. The reciprocator 1700A may be positioned between the ureteroscope and a valve (e.g., a Tuohy-Borst valve) gripping the ablation instrument 1250. Rotation of the rotating cam 1704 may cause the valve and corresponding ablation instrument 1250 to move as described in greater detail herein. In some examples, the rotating cam 1704 may be rotated manually (e.g., by an operator). In some examples, the rotating cam 1704 may be rotated automatically (e.g., operatively coupled to a motor).

The body portion 1702A can be configured to couple to the ablation instrument 1250 and to translate axially along a longitudinal axis of the body portion 1702A. The body portion 1702A can include a first end 1706 and a second end 1708 opposite the first end 1706, wherein the longitudinal axis extends between the first end 1706 and the second end 1708. The body portion 1702A can include a first body portion 1710 and a second body portion 1712. The first body portion 1710 can be positioned at the first end 1706 and the second body portion 1712 can be positioned at the second end 1708. In some examples, the second body portion 1712 may be a stationary portion. Accordingly, the first body portion 1710 may be configured to move relative to the second body portion 1712.

In some examples, the body portion 1702A may further include a shaft 1714 extending from the second body portion 1712 toward the first end 1706. The first body portion 1710 may be configured to translate about the shaft 1714.

In some examples, the body portion 1702A can further include a spring 1716. The spring 1716 can be a device configured to bias the first body portion 1710 to a neutral position. For example, the spring 1716 may be a helical wire configured to expand and compress in response to translation of the first body portion 1710. The spring 1716 may be positioned between the first body portion 1710 and the second body portion 1712 and disposed around the shaft 1714.

The first body portion 1710 can include one or more pins 1718. The one or more pins 1718 may extend radially outward from the first body portion 1710. In some examples, the pins 1718 may be configured to move along drive surface of the rotating cam 1704A. Movement of the pins 1718 along the drive surface of the rotating cam 1704A can act to translate the first body portion 1710 along the longitudinal axis.

The first body portion 1710 may further include a lumen 1720. The lumen 1720 may extend through the first body portion 1710. In some examples, the lumen 1720 may be configured to fit over the shaft 1714.

The first body portion 1710 may further include an engagement surface 1722. The engagement surface 1722 can be configured to secure the first end 1706 of the first body portion 1710 to another device. In some examples, the engagement surface 1722 may be threaded.

The rotating cam 1704A can include a first end 1724 and a second end 1726 opposite the first end 1724. The rotating cam 1704A may further include a drive surface 1728. The drive surface 1728 may be disposed along an annular surface of the rotating cam 1704A. In some examples, the drive surface 1728 may be disposed along an interior annular surface of the rotating cam 1704A. In some examples, the drive surface 1728 may be disposed along an exterior annular surface of the rotating cam 1704A. In some examples, the drive surface 1728 may be a shelf, a groove, and/or a slot. The drive surface 1728 may extend radially inward form an interior annular surface and/or radially outward from an exterior annular surface. A shelf may be a single surface. A groove and/or slot may be dual surfaces spaced from one another by the diameter of the pins 1718.

Additionally, the drive surface 1728 may be non-constant along an annular path. In some examples, the drive surface 1728 may extend at least partially in a longitudinal direction. For example, the drive surface 1728 may extend both annularly around the rotating cam 1704A and at least partially longitudinally. The longitudinal extension may be non constant. In some examples, the longitudinal extension may be directed toward the first end 1724 of the rotating cam 1704A at one or more locations of the annular path and may be directed toward the second end 1726 of the rotating cam 1704A at another one or more locations of the annular path. In some examples, the drive surface 1728 may provide a repeating pattern. For example, the drive surface 1728 may have multiple defined positions. The multiple defined positions can include a first position 1730, a second position 1732, and a third position 1734. The first position 1730 may be a position located axially between the second position 1732 and the third position 1734. The second position 1732 may be a position located axially proximal to the first end 1724. The third position 1734 may be a position located axially proximal to the second end 1726. The repeating pattern may include passing from the first position 1730 to the second position 1732, from the second position 1732 to the third position 1734, and from the third position 1734 back to the first position 1730. This pattern may be repeated one or more times.

As shown in FIGS. 27A-27B, in an assembled state, the rotating cam 1704A may be positioned radially outward of the body portion 1702A. The pins 1718 may extend radially outward from the body portion 1702A rest on the drive surface 1728 disposed on the interior annular surface of the rotating cam 1704A. As the rotating cam 1704A rotates, the pins 1718 may traverse the drive surface 1728 between the first position 1730, the second position 1732, and the third position 1734.

The first position 1730 can correspond to a neutral position of the body portion 1702A. In some examples, the neutral position of the body portion 1702A may correspond to a neutral position of a corresponding ablation instrument 1250. For example, the first position 1730 may correspond to a position where the distal tip of the ablation instrument 1250 is positioned within the choke 1640 of the ureteroscope.

The second position 1732 can correspond to a compressed position of the body portion 1702A wherein the first body portion 1710 is displaced closer to the second body portion 1712. In some examples, the compressed position of the body portion 1702A may correspond to a position where the ablation instrument 1250 is retracted within the ureteroscope. For example, the second position 1732 may correspond to a position where the distal tip of the ablation instrument 1250 is positioned proximal of the choke 1640. In some examples, the distal tip may be positioned 1-2 mm proximal of the choke 1640 in the second position 1732. Accordingly, the second position 1732 may allow biological objects 30 to pass through the aspiration port opening which may prevent biological objects 30 from occluding the aspiration port opening.

The third position 1734 can correspond to an extended position of the body portion 1702A wherein the first body portion 1710 is displaced away to the second body portion 1712. In some examples, the extended position of the body portion 1702A may correspond to a position where the ablation instrument 1250 is extended from the ureteroscope. For example, the third position 1734 may correspond to a position where the distal tip of the ablation instrument 1250 is positioned distal of the choke 1640. In some examples, the distal tip may be positioned 1-2 mm distal of the choke 1640 in the second position 1732. Accordingly, the third position 1734 may ensure that any hysteresis in the position of the ablation instrument 1250 is overcome prior to repositioning the ablation instrument 1250 in the neutral position.

Accordingly, a rotating cam 1704A may move the ablation instrument 1250 proximally back to the neutral position within the choke 1640. Proximally moving the ablation instrument 1250 may provide better repeatability in positioning the ablation instrument 1250 compared to distally moving the ablation instrument 1250 due to possible buckling with pushing the ablation instrument 1250.

FIG. 27C illustrates an example of a reciprocator 1700B. The reciprocator 1700B may be a variation of the reciprocator 1700A described above. The reciprocator 1700B may be similar to the reciprocator 1700A with the differences described herein. As shown in FIG. 27C, the rotating cam 1704B may not arranged along a common longitudinal axis as shown in FIG. 27A. Instead, the rotating cam 1704B may be radially offset and arranged along a longitudinal axis parallel to the longitudinal axis of the body portion 1702B. For example, the body portion 1702B may have a single pin 1718 extending radially outward from the first body portion 1710. The pin 1718 may be configured to engage a drive surface 1728 disposed along an exterior annular surface of the rotating cam 1704B. The rotating cam 1704B may include the same positions described herein with reference to FIGS. 27A and 27B. Additionally, the rotating cam 1704B may be operatively coupled with a motor 1736. The motor 1736 may run continuously during aspiration to assist with clearing obstructions. Accordingly, the obstructions may be removed instantaneously without requiring the operator to manually move the ablation instrument 1250. The frequency of each cycle can be controlled by the speed of the motor 1736.

FIG. 27D illustrates an example of a reciprocator 1700C. The reciprocator 1700C may be a variation of the reciprocator 1700A described above. The reciprocator 1700C may be similar to the reciprocator 1700A with the differences described herein. As shown in FIG. 27D, the rotating cam 1704C may be a pinion gear having a plurality of radially extending teeth instead of a drive surface 1728. The teeth may be identical and arranged parallel to a longitudinal axis of the rotating cam 1704C. The first body portion 1710 may include a rack 1738. The rack 1738 may be a linear gear having teeth instead of a pin 1718. The teeth may be identical and arranged orthogonally to the longitudinal axis of the body portion 1702C. The rotating cam 1704C may engage the rack 1738 to drive the first body portion 1710. As further shown in FIG. 27D, the rotating cam 1704C may be operatively coupled to a motor 1736. In some examples, the motor 1736 may be a servo motor. Accordingly, a plurality of positions may be accurately provided via the encoder of the servo motor to position the ablation instrument 1250 as desired. The reciprocator 1700C may provide the ablation instrument 1250 in a plurality of variable positions and speeds and may not be limited to the positioned described herein with reference to FIGS. 27A-27C. Additionally, The motor 1736 may run continuously during aspiration to assist with clearing obstructions. Accordingly, the obstructions may be removed instantaneously without requiring the operator to manually move the ablation instrument 1250. The frequency of each cycle can be controlled by the speed of the motor 1736. In some examples, the motor 1736 may be activated on-demand by the operator. In some examples, the motor 1736 may be activated on-demand of a software program. For example, a software program may include a video input to visually inspect the ureteroscope and monitor for occlusions.

### Example Implementations

Examples of the implementations of the present disclosure can be described in view of the following example clauses. The features recited in the below example implementations can be combined with additional features disclosed herein. Furthermore, additional inventive combinations of features are disclosed herein, which are not specifically recited in the below example implementations, and which do not include the same features as the specific implementations below. For sake of brevity, the below example implementations do not identify every inventive aspect of this disclosure. The below example implementations are not intended to identify key features or essential features of any subject matter described herein. Any one or more features of one of the example clauses listed below can be combined by any of the one or more features of any one or more other example clauses listed below or any of the features described herein.

Clause 1. A system for use in a removal procedure of a biological object from an anatomical structure, the system comprising: a catheter body comprising a proximal end and a distal end configured to be inserted into the anatomical structure, the catheter body comprising one or more openings configured to receive the biological object; an aspiration lumen positioned at least partially in the catheter body, the aspiration lumen being in fluid communication with the one or more openings, and the aspiration lumen being configured to aspirate and transport at least a portion of the biological object from the one or more openings toward a proximal end of the aspiration lumen; and a supply lumen configured to transport a liquid from a liquid source to the distal end of the catheter body, the supply lumen comprising a first portion extending at least between the proximal end and the distal end of the catheter body and a second portion at the distal end of the catheter body, wherein a longitudinal axis of the second portion oriented at an angle relative to a longitudinal axis of first portion such that the liquid is ejected from the supply lumen at an angle relative to the longitudinal axis of the first portion greater than 90 degrees and less than 180 degrees, and wherein the second portion configured to eject the liquid outside the supply lumen and create vacuum via a jet pump effect to attract the biological object toward the one or more openings.

Clause 2. The system of clause 1, wherein the second portion is configured to eject the liquid along a helical path within the aspiration lumen.

Clause 3. The system of any one of clauses 1 and 2, further comprising a plurality of vent port openings formed in the catheter body and fluidly connected to the aspiration lumen, wherein the plurality of vent port openings is configured to aspirate a portion of the biological object.

Clause 4. The system of any one of clauses 1-3, further comprising a plurality of vent port openings formed in the catheter body and fluidly connected to the aspiration lumen, wherein the plurality of vent port openings is configured to resupply into the anatomical structure at least some of the liquid ejected from the supply lumen.

Clause 5. The system of any one of clauses 1-4, wherein at least one opening of the one or more openings is positioned at a tip of the distal end of the catheter body.

Clause 6. The system of any one of clauses 1-5, wherein at least one opening of the one or more openings is positioned on a side of the catheter body.

Clause 7. The system of any one of clauses 1-6, wherein the supply lumen is positioned at least partially in the aspiration lumen.

Clause 8. The system of any one of clause 1-7, wherein the supply lumen is positioned along an inner wall within the catheter body without obstructing a central passage within the catheter body.

Clause 9. The system of any one of clauses 1-8, wherein the supply lumen comprises a constriction.

Clause 10. The system of any one of clauses 1-9, further comprising insulating material configured to insulate the supply lumen from liquid being aspirated through the aspiration lumen, the insulating material facilitating maintenance of a temperature in the anatomical structure at or below a temperature threshold indicative of safe operating temperature.

Clause 11. The system of any one of clauses 1-10, further comprising an endoscope configured to support the catheter body.

Clause 12. The system of any one of clauses 1-11, further comprising an ablation device configured to be delivered through the aspiration lumen.

Clause 13. The system of any one of clauses 1-12, wherein a portion of the distal end of catheter body is transparent at wavelengths corresponding to visible light and wavelengths corresponding to laser energy emitted by an ablation device.

Clause 14. The system of any one of clauses 1-13, further comprising a flow regulator configured to be in fluid communication with the proximal end of the aspiration lumen.

Clause 15. The system of clause 14, wherein the flow regulator is configured to regulate a fluid flow through the aspiration lumen.

Clause 16. The system of any one of clauses 14-15, wherein the flow regulator comprises a vacuum source.

Clause 17. The system of any one of clauses 14-16, wherein the flow regulator comprises a valve.

Clause 18. The system of any one of clauses 1-17, further comprising a flow controller configured to regulate a fluid flow through the aspiration lumen.

Clause 19. The system of clause 18, wherein the flow controller is further configured to regulate pressure within the system.

Clause 20. The system of any one of clauses 18-19, further comprising at least one pressure sensor configured to monitor pressure in the aspiration lumen and wherein the flow controller is configured to regulate pressure using the monitored pressure.

Clause 21. The system of any one of clauses 18-20, wherein the flow controller is configured to regulate pressure in at least one of the aspiration lumen or the supply lumen.

Clause 22. The system of any one of clauses 1-21, further comprising the liquid source configured to be in fluid communication with the supply lumen and configured to supply the liquid to the supply lumen.

Clause 23. The system of any one of clauses 1-22, wherein the liquid source is a liquid reservoir.

Clause 24. The system of any one of clauses 1-23, further comprising a pump configured to supply the liquid from the liquid source to the supply lumen.

Clause 25. The system of any one of clauses 1-24, wherein ejecting the liquid outside the supply lumen creates the vacuum at the one or more openings of the catheter body.

Clause 26. The system of any one of clauses 1-25, wherein a distal tip of the catheter body is formed from fluoropolymers.

Clause 27. The system of any one of clauses 1-26, wherein the biological object is positioned in a urinary tract.

Clause 28. The system of any one of clauses 1-27, wherein the biological object is a urinary calculus.

Clause 29. The system of any one of clauses 1-27, wherein the anatomical structure is a urinary tract.

Clause 30. The system of any one of clauses 1-28, wherein the anatomical structure is a kidney.

Clause 31. A system for use a removal procedure of a biological object from an anatomical structure, the system comprising: a catheter body comprising a proximal end and a distal end configured to be inserted into the anatomical structure, the catheter body comprising one or more openings configured to the biological object; an aspiration lumen positioned at least partially in the catheter body, the aspiration lumen being in fluid communication with the one or more openings, and the aspiration lumen being configured to aspirate and transport at least the biological object from the one or more openings toward a proximal end of the aspiration lumen; and a supply lumen configured to transport a liquid from a liquid source to the distal end of the catheter body, the supply lumen comprising a first portion extending at least between the proximal end and the distal end of the catheter body and a second portion at the distal end of the catheter body that redirects a flow of the liquid at least partially toward the proximal end of the catheter body, and the second portion configured to eject the liquid outside the supply lumen and create vacuum via a to attract the biological object toward the one or more openings.

Clause 32. The system of clause 31, wherein the second portion is configured to eject the liquid along a helical path within the aspiration lumen.

Clause 33. The system of any one of clauses 31-32, further comprising a plurality of vent port openings formed in the catheter body and fluidly connected to the aspiration lumen, wherein the plurality of vent port openings is configured to aspirate a portion of the biological object.

Clause 34. The system of any one of clauses 31-33, further comprising a plurality of vent port openings formed in the catheter body and fluidly connected to the aspiration lumen, wherein the plurality of vent port openings is configured to resupply into the anatomical structure at least some of the liquid ejected from the supply lumen.

Clause 35. The system of any one of clauses 31-34, wherein the one or more openings is positioned at a tip of the distal end of the catheter body.

Clause 36. The system of any one of clauses 31-35, wherein at least one opening of the one or more openings is positioned on a side of the catheter body.

Clause 37. The system of any one of clauses 31-36, wherein the supply lumen is positioned at least partially in the aspiration lumen.

Clause 38. The system of any one of clauses 31-37, wherein the supply lumen is positioned along an inner wall within the catheter body without obstructing a central passage within the catheter body.

Clause 39. The system of any one of clauses 31-38, wherein the supply lumen comprises a constriction.

Clause 40. The system of any one of clauses 31-39, further comprising insulating material configured to insulate the supply lumen from liquid being aspirated through the aspiration lumen, the insulating material facilitating maintenance of a temperature in the anatomical structure at or below a temperature threshold indicative of safe operating temperature.

Clause 41. The system of any one of clauses 31-40, further comprising an endoscope configured to support the catheter body.

Clause 42. The system of any one of clauses 31-41, further comprising an ablation device configured to be delivered through the aspiration lumen.

Clause 43. The system of any one of clauses 31-42, wherein a portion of the distal end of the catheter body is transparent at wavelengths corresponding to visible light and wavelengths corresponding to laser energy emitted by an ablation device.

Clause 44. The system of any one of clauses 31-43, further comprising a flow regulator configured to be in fluid communication with the proximal end of the aspiration lumen.

Clause 45. The system of clause 44, wherein the flow regulator is configured to regulate a fluid flow through the aspiration lumen.

Clause 46. The system of any one of clauses 44-45, wherein the flow regulator comprises a vacuum source.

Clause 47. The system of any one of clauses 44-46, wherein the flow regulator comprises a valve.

Clause 48. The system of any one of clauses 31-47, further comprising a flow controller configured to regulate a fluid flow through the aspiration lumen.

Clause 49. The system of clause 48, wherein the flow controller is further configured to regulate pressure within the system.

Clause 50. The system of any one of clauses 48-49, further comprising at least one pressure sensor configured to monitor pressure in the aspiration lumen and wherein the flow controller is configured to regulate pressure using the monitored pressure.

Clause 51. The system of any one of clauses 48-50, wherein the flow controller is configured to regulate pressure in at least one of the aspiration lumen or the supply lumen.

Clause 52. The system of any one of clauses 31-51, wherein the liquid source is configured to be in fluid communication with the supply lumen and configured to supply the liquid to the supply lumen.

Clause 53. The system of any one of clauses 31-52, wherein the liquid source is a liquid reservoir.

Clause 54. The system of any one of clauses 31-53, further comprising a pump configured to supply the liquid from the liquid source to the supply lumen.

Clause 55. The system of any one of clauses 31-54, wherein ejecting the liquid outside the supply lumen creates the vacuum at the one or more openings of the catheter body.

Clause 56. The system of any one of clauses 31-55, wherein a distal tip of the catheter body is formed from fluoropolymers.

Clause 57. The system of any one of clauses 31-56, wherein the biological object is positioned in a urinary tract.

Clause 58. The system of any one of clauses 31-57, wherein the biological object is a urinary calculus.

Clause 59. The system of any one of clauses 31-58, wherein the anatomical structure is a urinary tract.

Clause 60. The system of any one of clauses 31-59, wherein the anatomical structure is a kidney.

Clause 61. A system for use in a removal procedure of a biological object from an anatomical structure, the system comprising: a catheter body comprising a proximal end and a distal end configured to be inserted into the anatomical structure, the catheter body comprising one or more openings configured to the biological object; an aspiration lumen positioned at least partially in the catheter body in fluid communication with the one or more openings, the aspiration lumen configured to be in fluid communication with a vacuum source, the vacuum source being configured to supply vacuum at a first vacuum level to the proximal end of the aspiration lumen and to facilitate aspiration of the biological object from the one or more openings toward the proximal end of the aspiration lumen; and a supply lumen configured to transport a liquid from a liquid source to the distal end of the catheter body, the supply lumen comprising a first portion extending at least partially between the proximal end and the distal end of the catheter body and a second portion at the distal end of the catheter body configured to redirect and eject a flow of the liquid at least partially toward the proximal end of the catheter body to supply vacuum at a second vacuum level at the one or more openings so as to attract the biological object towards the one or more openings.

Clause 62. The system of clause 61, wherein ejecting the liquid from the supply lumen into the catheter body creates a vacuum via entrainment.

Clause 63. The system of any one of clauses 61-62, wherein the second vacuum level attracts the biological object towards the one or more openings via entrainment.

Clause 64. The system of any one of clauses 61-63, wherein the supply lumen comprises a first diameter and the catheter body comprises a second diameter greater than the first diameter, wherein the flow of the liquid is ejected from a volume of the supply lumen with the first diameter into a volume of the catheter body with the second diameter.

Clause 65. The system of any one of clauses 61-64, wherein the vacuum source is configured to regulate fluid flow through the catheter body.

Clause 66. The system of any one of clauses 61-65, wherein the biological object is positioned in a urinary tract.

Clause 67. The system of any one of clauses 61-66, wherein the biological object is a urinary calculus.

Clause 68. The system of any one of clauses 61-67, wherein the anatomical structure is a urinary tract.

Clause 69. The system of any one of clauses 61-68, wherein the anatomical structure is a kidney.

Clause 70. A method of removing a biological object from an anatomical structure, the method comprising: creating a vacuum with a catheter in the anatomical structure, the catheter including a proximal end and a distal end, an aspiration lumen positioned in a catheter body of the catheter, and a supply lumen configured to transport a liquid from a liquid source to the distal end of the catheter, the supply lumen comprising a bend at the distal end of the catheter, the bend being greater than 90 degrees and less than 180 degrees, wherein the vacuum is created at one or more openings in the catheter body to attract a biological object toward the one or more openings, the vacuum being created, via a jet pump effect, by transporting the liquid through the supply lumen and ejecting the liquid into the aspiration lumen; and aspirating at least a portion of the biological object through the aspiration lumen towards the proximal end of the aspiration lumen.

Clause 71. The method of clause 70, wherein the biological object comprises a urinary calculus, and wherein the method further comprises fragmenting the urinary calculus by ablation and aspirating at least one fragment of the urinary calculus through the aspiration lumen toward the proximal end of the aspiration lumen.

Clause 72. The method of any one of clauses 70-71, wherein the vacuum creates a vortex within the aspiration lumen adjacent to the one or more openings.

Clause 73. The method of one of clauses 70-72, further comprising regulating flow through the aspiration lumen.

Clause 74. The method of clause 73, wherein regulating flow through the aspiration lumen comprises applying negative pressure to the proximal end of the catheter or operating a valve.

Clause 75. The method of any one of clauses 70-74, further comprising ejecting the liquid from the supply lumen through a plurality of vent port openings formed in the catheter body.

Clause 76. The method of any one of clauses 70-75, wherein the one or more openings are positioned at a tip of the distal end of the catheter body.

Clause 77. The method of any one of clauses 70-76, wherein the catheter is delivered to the anatomical structure through a working lumen of an endoscope, and wherein the anatomical structure comprises a urinary tract.

Clause 78. The method of any one of clauses 70-77, wherein creating the vacuum at the one or more openings in the catheter body comprises transporting the liquid through the supply lumen at a first flow rate and ejecting the liquid from the supply lumen into the catheter body comprising another liquid flowing at a second flow rate lesser than the first flow rate.

Clause 79. The method of any one of clauses 70-78, wherein the supply lumen comprises a first diameter and the catheter body comprises a second diameter greater than the first diameter.

Clause 80. The method of any one of clauses 70-79, further comprising irrigating the anatomical structure via an irrigation lumen in fluid communication with a second liquid source.

Clause 81. The method of clause 80, wherein the second liquid source is configured to be raised at a height relative to the catheter to provide flow in the irrigation lumen.

Clause 82. The method of clause 81, wherein the height is selected to regulate pressure within the anatomical structure.

Clause 83. The method of any one of clauses 80-82, wherein a flow in the irrigation lumen is provided without using a pump.

Clause 84. The method of clause 83, wherein the flow in the irrigation lumen results from a gravity fed pressure.

Clause 85. The method of clause 84, wherein the gravity fed pressure is a constant pressure head configured to regulate pressure within the anatomical structure.

Clause 86. The method of any one of clauses 70-85, wherein the biological object is positioned in a urinary tract.

Clause 87. The method of any one of clauses 70-86, wherein the biological object is a urinary calculus.

Clause 88. The method of any one of clauses 70-87, wherein the anatomical structure is a urinary tract.

Clause 89. The method of any one of clauses 70-88, wherein the anatomical structure is a kidney.

Clause 90. A method of removing a biological object from an anatomical structure, the method comprising: creating a vacuum with a catheter in the anatomical structure, the catheter including a proximal end and a distal end, an aspiration lumen positioned in a catheter body of the catheter, and a supply lumen configured to transport a liquid from a liquid source to the distal end of the catheter, the supply lumen comprising a first portion extending at least between the proximal end and the distal end of the catheter body of the catheter and a second portion at the distal end of the catheter body of the catheter that redirects a flow of the liquid at least partially toward the proximal end of the catheter body, wherein the vacuum is created at one or more openings in the catheter body to attract the biological object toward the one or more openings, the vacuum being created, via a jet pump effect, by transporting the liquid through the supply lumen and ejecting the liquid into the aspiration lumen; and aspirating at least a portion of the biological object through the aspiration lumen towards the proximal end of the catheter.

Clause 91. The method of clause 90, further comprising breaking the biological object into a plurality of fragments with ablation.

Clause 92. The method of any one of clauses 90-91, wherein ablation is provided by an ablation device positioned within the aspiration lumen.

Clause 93. The method of any one of clauses 90-92, wherein the vacuum creates a vortex within the aspiration lumen adjacent to the one or more openings.

Clause 94. The method of any one of clauses 90-93, further comprising applying negative pressure to the proximal end of the catheter to aspirate the portion of the biological object.

Clause 95. The method of any one of clauses 90-94, further comprising applying negative pressure to the proximal end of the catheter or operating a valve to regulate fluid flow through the aspiration lumen.

Clause 96. The method of any one of clauses 90-95, further comprising ejecting the liquid from the supply lumen through a plurality of vent port openings formed in the catheter body.

Clause 97. The method of any one of clauses 90-96, wherein the one or more openings are positioned at a tip of the distal end of the catheter body.

Clause 98. The method of any one of clauses 90-97, wherein the catheter is delivered to the anatomical structure through a working channel.

Clause 99. The method of clause 98, wherein the working channel is part an endoscope.

Clause 100. The method of any one of clauses 90-99, wherein creating the vacuum at the one or more openings comprises transporting the liquid through the supply lumen at a first flow rate and ejecting the liquid from the supply lumen into the catheter body comprising another liquid flowing at a second flow rate lesser than the first flow rate.

Clause 101. The method of any one of clauses 90-100, wherein the supply lumen comprises a first diameter and the catheter body comprises a second diameter greater than the first diameter.

Clause 102. The method of any one of clauses 90-101, further comprising irrigating the anatomical structure via an irrigation lumen in fluid communication with a second liquid source.

Clause 103. The method of clause 102, wherein the second liquid source provides a gravity fed pressure to the irrigation lumen.

Clause 104. The method of clause 103, wherein the gravity fed pressure is a constant pressure head configured to regulate pressure within the anatomical structure.

Clause 105. The method of clause 104, wherein the biological object is a urinary calculus and the anatomical structure is a urinary tract.

Clause 106. The method of any one of clauses 90-105, wherein the biological object is positioned in a urinary tract.

Clause 107. The method of any one of clauses 90-106, wherein the biological object is a urinary calculus.

Clause 108. The method of any one of clauses 90-107, wherein the anatomical structure is a urinary tract.

Clause 109. The method of any one of clauses 90-108, wherein the anatomical structure is a kidney.

Clause 110. A method of removing a biological object from an anatomical structure, the method comprising: creating a vacuum with a catheter in the anatomical structure, the catheter including a proximal end, a distal end, an aspiration lumen positioned in a catheter body of the catheter, and a supply lumen positioned in the aspiration lumen, wherein the vacuum is created at one or more openings in the catheter body of the catheter to attract a biological object the one or more openings, the vacuum being created, via a vortex effect, by ejecting a liquid from the supply lumen along a helical path within the catheter body; and aspirating at least a portion of the biological object through the one or more openings in the catheter body and transporting the portion of the biological object along the aspiration lumen toward the proximal end of the catheter.

Clause 111. The method of clause 110, wherein the biological object is a urinary calculus and the anatomical structure is a urinary tract.

Clause 112. The method of any one of clauses 110-111, wherein the supply lumen comprises a bend at the distal end of the catheter, the bend being greater than 90 degrees and less than 180 degrees.

Clause 113. The method of any one of clauses 110-112, wherein the supply lumen comprises a first portion extending at least between the proximal end and the distal end of the catheter body and a second portion at the distal end of the catheter body that redirects a flow of the liquid at least partially toward the proximal end of the catheter body.

Clause 114. The method of any one of clauses 110-113, wherein the vortex effect is created by ejecting the liquid from the supply lumen at an angle relative to a central axis of the catheter.

Clause 115. The method of any one of clauses 110-114, wherein the supply lumen is configured to output the liquid at an angle relative to a central cross-sectional axis of the catheter.

Clause 116. The method of any one of clauses 110-115, wherein the supply lumen is configured to output the liquid at an angle relative to a longitudinal axis of the catheter.

Clause 117. The method of any one of clauses 110-116, wherein the liquid contacts an inner surface of the catheter at a non-normal angle.

Clause 118. The method of any one of clauses 110-117, wherein the vortex effect creates a liquid flow within the catheter that follows an annular path defined by an inner surface of the catheter along a longitudinal length of the catheter.

Clause 119. The method of any one of clauses 110-118, wherein the biological object is broken with an ablation device.

Clause 120. The method of clause 119, wherein the ablation device is positioned within the aspiration lumen.

Clause 121. The method of any one of clauses 110-120, wherein the vacuum creates a vortex within the aspiration lumen adjacent the one or more openings.

Clause 122. The method of any one of clauses 110-121, further comprising applying negative pressure to the proximal end of the catheter to aspirate at least a portion of the biological object.

Clause 123. The method of any one of clauses 110-122, further comprising applying negative pressure to the proximal end of the catheter or operating a valve to regulate fluid flow through the aspiration lumen.

Clause 124. The method of any one of clauses 110-123, further comprising ejecting the liquid from the supply lumen through a plurality of vent port openings formed in the catheter body.

Clause 125. The method of any one of clauses 110-124, wherein the one or more openings is positioned at a tip of the distal end of the catheter body.

Clause 126. The method of any one of clauses 110-125, wherein the catheter is delivered to the anatomical structure through a working channel.

Clause 127. The method of clause 126, wherein the working channel is part of an endoscope.

Clause 128. The method of any one of clauses 110-127, wherein creating the vacuum at the one or more openings comprises transporting the liquid through the supply lumen at a first flow rate and ejecting the liquid from the supply lumen into the catheter body comprising another liquid flowing at a second flow rate lesser than the first flow rate.

Clause 129. The method of any one of clauses 110-128, wherein the supply lumen comprises a first diameter and the catheter body comprises a second diameter greater than the first diameter.

Clause 130. The method of any one of clauses 110-129, further comprising irrigating the anatomical structure via an irrigation lumen in fluid communication with a second liquid source.

Clause 131. The method of clause 130, wherein the second liquid source provides a gravity fed pressure to the irrigation lumen.

Clause 132. The method of clause 131, wherein the gravity fed pressure is a constant pressure head configured to regulate pressure within the anatomical structure.

Clause 133. A method of removing a biological object from an anatomical structure, the method comprising: creating a vacuum with a catheter in the anatomical structure, the catheter including a proximal end and a distal end, an aspiration lumen positioned in a catheter body, and a supply lumen positioned in the aspiration lumen, the vacuum being created at one or more openings in the catheter body to attract a biological object toward the one or more openings by ejecting a liquid from an end of the supply lumen into the aspiration lumen; breaking the biological object captured and retained at the one or more openings in the catheter body into a plurality of fragments; aspirating at least some of the plurality of fragments of the biological object through the one or more openings in the catheter body and transporting at least some of the plurality of fragments of the biological object along the aspiration lumen toward the proximal end of the catheter; and aspirating through a plurality of vent port openings formed in the catheter body at least some of the plurality of fragments of the biological object.

Clause 134. The method of clause 133, wherein aspirating at least some of the plurality of fragments of the biological object through the one or more of the plurality of vent port openings improves visibility of the anatomical structure.

Clause 135. The method of any one of clauses 133-134, further comprising ejecting the liquid from the catheter body through one or more of the plurality of vent port openings.

Clause 136. The method of any one of clauses 133-135, wherein the vacuum is created, via a jet pump effect.

Clause 137. The method of any one of clauses 133-136, wherein the liquid is ejected from the distal end of the supply lumen into the aspiration lumen.

Clause 138. The method of any one of clauses 133-137, wherein ejecting the liquid comprises ejecting the liquid at least partially toward the proximal end of the catheter body.

Clause 139. The method of any one of clauses 133-138, wherein the biological object is positioned in a urinary tract.

Clause 140. The method of any one of clauses 133-139, wherein the biological object is a urinary calculus.

Clause 141. The method of any one of clauses 133-140, wherein the anatomical structure is a urinary tract.

Clause 142. The method of any one of clauses 133-141, wherein the anatomical structure is a kidney.

Clause 143. A system for use in a removal procedure of a biological object from an anatomical structure, the system comprising: an access sheath comprising an irrigation lumen and a seal positioned at a proximal end of the irrigation lumen, the access sheath being configured to be inserted into the anatomical structure and to transport a first liquid from a first liquid source to the anatomical structure through the irrigation lumen; an endoscope configured to be inserted into the anatomical structure through the seal of the access sheath, the endoscope comprising a working channel extending between a proximal end of the endoscope and a distal end of the endoscope; and a catheter configured to be inserted through the working channel of the endoscope, the catheter comprising: a catheter body comprising one or more openings configured to receive the biological object; an aspiration lumen positioned at least partially in the catheter body, the aspiration lumen being in fluid communication with the one or more openings, and the aspiration lumen being configured to aspirate and transport at least the biological object from the one or more openings toward a proximal end of the aspiration lumen; and a supply lumen configured to transport a second liquid from a second liquid source to the distal end of the catheter body, the supply lumen comprising a first portion and a second portion configured to eject the second liquid outside the supply lumen, the second portion being positioned at the distal end of the catheter body and configured to redirect the second liquid at least partially toward the proximal end of the catheter body, wherein ejecting the second liquid from an outlet of the supply lumen creates a vacuum to attract the biological object toward the one or more openings.

Clause 144. The system of clause 143, wherein ejecting the second liquid from the outlet of the supply lumen creates a vacuum via a Venturi effect to attract the biological object toward the one or more openings.

Clause 145. The system of any one of clauses 143-144, wherein ejecting the second liquid from the outlet of the supply lumen creates a vacuum via a jet pump effect to attract the biological object toward the one or more openings.

Clause 146. The system of any one of clauses 143-145, wherein the second portion is configured to eject the second liquid along a helical path within the aspiration lumen.

Clause 147. The system of any one of clauses 143-146, further comprising a plurality of vent port openings formed in the catheter body and fluidly connected to the aspiration lumen, wherein the plurality of vent port openings is configured to aspirate a portion of the biological object.

Clause 148. The system of any one of clauses 143-147, further comprising a plurality of vent port openings formed in the catheter body and fluidly connected to the aspiration lumen, wherein the plurality of vent port openings is configured to resupply into the anatomical structure at least some of the second liquid ejected from the supply lumen.

Clause 149. The system of any one of clauses 143-148, wherein at least one opening the one or more openings is positioned at a tip of the distal end of the catheter body.

Clause 150. The system of any one of clauses 143-149, wherein at least one opening of the one or more openings is positioned on a side of the catheter body.

Clause 151. The system of any one of clauses 143-150, wherein the supply lumen is positioned at least partially in the aspiration lumen.

Clause 152. The system of any one of clauses 143-151, wherein the supply lumen is positioned along an inner wall within the catheter body without obstructing a central passage within the catheter body.

Clause 153. The system of any one of clauses 143-152, wherein the second portion of the supply lumen is fully contained within the aspiration lumen.

Clause 154. The system of any one of clauses 143-153, wherein the second portion comprises a constriction.

Clause 155. The system of any one of clauses 143-154, further comprising insulating material configured to insulate the supply lumen from liquid being aspirated through the aspiration lumen, the insulating material facilitating maintenance of a temperature in the anatomical structure at or below a temperature threshold indicative of safe operating temperature.

Clause 156. The system of any one of clauses 143-155, further comprising an ablation device configured to be delivered through the aspiration lumen.

Clause 157. The system of any one of clauses 143-156, wherein a portion of the distal end of catheter body is transparent at wavelengths corresponding to visible light and wavelengths corresponding to laser energy emitted by an ablation device.

Clause 158. The system of any one of clauses 143-157, further comprising a flow regulator configured to be in fluid communication with the proximal end of the aspiration lumen.

Clause 159. The system of clause 158, wherein the flow regulator is configured to regulate a fluid flow through the aspiration lumen.

Clause 160. The system of any one of clauses 158-159, wherein the flow regulator comprises a vacuum source.

Clause 161. The system of any one of clauses 158-160, wherein the flow regulator comprises a valve.

Clause 162. The system of any one of clauses 143-161, further comprising a flow controller configured to regulate a fluid flow through the aspiration lumen.

Clause 163. The system of clause 162, wherein the flow controller is further configured to regulate pressure within the system.

Clause 164. The system of any one of clauses 162-163, further comprising at least one pressure sensor configured to monitor pressure in the aspiration lumen and wherein the flow controller is configured to regulate pressure using the monitored pressure.

Clause 165. The system of any one of clauses 162-164, wherein the flow controller is configured to regulate pressure in at least one of the aspiration lumen or the supply lumen.

Clause 166. The system of any one of clauses 143-165, further comprising the second liquid source configured to be in fluid communication with the supply lumen and configured to supply the second liquid to the supply lumen.

Clause 167. The system of any one of clauses 143-166, wherein the second liquid source is a liquid reservoir.

Clause 168. The system of any one of clauses 143-167, further comprising a pump configured to supply the second liquid from the second liquid source to the supply lumen.

Clause 169. The system of any one of clauses 143-168, wherein ejecting the second liquid outside the supply lumen creates the vacuum at the one or more openings of the catheter body.

Clause 170. The system of any one of clauses 143-169, wherein a distal tip of the catheter body is formed from fluoropolymers.

Clause 171. The system of any one of clauses 143-170, wherein the first liquid source is configured to be raised at a height relative to the catheter to provide an irrigation flow in the irrigation lumen.

Clause 172. The system of clause 171, wherein the height is selected to regulate pressure within the anatomical structure.

Clause 173. The system of any one of clauses 143-171, wherein a flow in the irrigation lumen is provided without using a pump.

Clause 174. The system of clause 173, wherein the irrigation flow in the irrigation lumen results from a gravity fed pressure.

Clause 175. The system of clause 174, wherein the gravity fed pressure is a constant pressure head configured to regulate pressure within the anatomical structure.

Clause 176. The system of any one of clauses 143-175, wherein the biological object is positioned in a urinary tract.

Clause 177. The system of any one of clauses 143-176, wherein the biological object is a urinary calculus.

Clause 178. The system of any one of clauses 143-177, wherein the anatomical structure is a urinary tract.

Clause 179. The system of any one of clauses 143-178, wherein the anatomical structure is a kidney.

Clause 180. The system of any one of clauses 1-69 and 143-179, wherein the supply lumen comprises a plurality of liquid supply lumens.

Clause 182. The system of any one of clauses 1-69 and 143-181, further comprising a cup, a choke, and a working channel extension.

Clause 183. The system of any one of clauses 1-69 and 143-182, wherein the supply lumen is configured to eject the liquid outside the supply lumen, wherein ejecting the liquid forms a low-pressure zone within the system.

Clause 184. The system of any one of clauses 1-69 and 143-183, wherein a suction force is generated for attracting biological objects toward the aspiration lumen.

Clause 185. The system of any one of clauses 1-69 and 143-183, wherein a suction force between about 0.65 mN and 4.02 N is generated at an aspiration port opening.

Clause 186. The system of any one of clauses 1-69 and 143-184, wherein a flow rate through an aspiration port opening is the same as a flow rate through a vent port opening.

Clause 187. The system of any one of clauses 1-69 and 143-184, further comprising a reciprocator.

Clause 188. The system of clause 187, wherein the reciprocator comprises a body portion and a rotating cam, wherein rotation of the rotating cam axially moves a portion of the body portion.

Clause 189. The system of any one of clauses 187-188, wherein a movement of the reciprocator axially moves an ablation instrument positioned within the aspiration lumen.

Clause 190. The system of any one of clauses 187-188, wherein the body portion comprises a first body portion and a second body portion, wherein the first body portion is configured to move axially relative to the second body portion via rotation of a rotating cam.

Clause 191. The system of any one of clauses 1-69 and 143-190, wherein the supply lumen is configured to eject the liquid outside the supply lumen from a plurality of outlets.

### Other Variations

While certain examples have been described in the context of ureteroscopy, the approaches, systems, devices, and methods described herein can be used for any medical procedure that utilizes a catheter, such as any medical procedure directed to removing a biological object from an anatomical structure within the body. While certain examples have been described in the context of removing solid deposits, the approaches described herein can be used for removing any biological object including, without limitation, blood clots, tumors, tissue samples, and urinary or fragmented urinary calculi such as bladder stones, ureter stones, and kidney stones, which may not necessarily be a solid, from any location internal to a subject including, without limitation, a kidney, a bladder, a heart, a colon, a duodenum, an ileum, a jejunum, a stomach, an esophagus, an intestine, a mouth, a liver, a lung, a pancreas, a spleen, a lymph node, a (blood) vessel, a gland, an ear canal, a urethra, a uterus, a gallbladder, an ovary, or a nasal cavity.

The foregoing description details certain examples of the systems, devices, and methods disclosed herein. It will be appreciated, however, that no matter how detailed the foregoing appears in text, the systems, devices, and methods can be practiced in many ways. It should be noted that the use of particular terminology when describing certain features or aspects of the disclosure should not be taken to imply that the terminology is being re-defined herein to be restricted to including any specific characteristics of the features or aspects of the technology with which that terminology is associated.

It will be appreciated by those skilled in the art that various modifications and changes may be made without departing from the scope of the described technology. Such modifications and changes are intended to fall within the scope of the examples. It will also be appreciated by those of skill in the art that parts included in one example are interchangeable with other examples; one or more parts from a depicted example can be included with other depicted examples in any combination. For example, any of the various components described herein and/or depicted in the Figures may be combined, interchanged or excluded from other examples.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

Conditional language used herein, such as, among others, “can,” “could”, “might,” “may,” “e.g.,” and the like, unless specifically stated otherwise, or otherwise understood within the context as used, is generally intended to convey that certain implementation include, while other implementations do not include, certain features, elements and/or states. Thus, such conditional language is not generally intended to imply that features, elements and/or states are in any way required for one or more implementations or that one or more implementations necessarily include logic for deciding, with or without author input or prompting, whether these features, elements and/or states are included or are to be performed in any particular implementation. The term “each,” as used herein, in addition to having its ordinary meaning, can mean any subset of a set of elements to which the term “each” is applied.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to examples containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All references cited herein are incorporated herein by reference in their entirety. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, or within less than 0.01% of the stated value.

It is noted that some examples above may be described as a process, which is depicted as a flowchart, a flow diagram, a structure diagram, or a block diagram. Although a flowchart may describe the operations as a sequential process, many of the operations can be performed in parallel, or concurrently, and the process can be repeated. In addition, the order of the operations may be rearranged. A process is terminated when its operations are completed. A process may correspond to a method, a function, a procedure, a subroutine, a subprogram, etc. When a process corresponds to a software function, its termination corresponds to a return of the function to the calling function or the main function.

Various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific examples disclosed above may be combined in different ways to form additional implementations, all of which fall within the scope of the present disclosure.

The above description discloses several methods and materials of the present disclosure. This disclosure is susceptible to modifications in the methods and materials, as well as alterations in the fabrication methods and equipment. Such modifications will become apparent to those skilled in the art from a consideration of this disclosure or practice of the examples disclosed herein. Consequently, it is not intended that this disclosure be limited to the specific examples disclosed herein, but that it covers all modifications and alternatives coming within the true scope and spirit of the disclosure as embodied in the attached claims.

1. A method of removing a biological object from an anatomical structure, the method comprising:
creating a vacuum with a catheter in the anatomical structure, the catheter including a proximal end and a distal end, an aspiration lumen positioned in a catheter body of the catheter, and a supply lumen configured to transport a liquid from a liquid source to the distal end of the catheter, the supply lumen comprising a first portion extending at least between the proximal end and the distal end of the catheter body of the catheter and a second portion at the distal end of the catheter body of the catheter that redirects a flow of the liquid at least partially toward the proximal end of the catheter body, wherein the vacuum is created at one or more distal openings in the catheter body to attract the biological object toward the one or more distal openings, the vacuum being created by transporting the liquid through the supply lumen and ejecting the liquid into the aspiration lumen; and
aspirating at least a portion of the biological object through the aspiration lumen towards the proximal end of the catheter.

2. The method of claim 1, further comprising breaking the biological object into a plurality of fragments with ablation.

3. The method of claim 1, wherein the vacuum creates a vortex within the aspiration lumen adjacent to the one or more distal openings.

4. The method of claim 1, further comprising applying negative pressure to the proximal end of the catheter to aspirate the portion of the biological object.

5. The method of claim 1, further comprising regulating fluid flow through the aspiration lumen.

6. The method of claim 1, further comprising ejecting the liquid from the supply lumen through a plurality of vent port openings formed in the catheter body.

7. The method of claim 1, wherein creating the vacuum at the one or more distal openings comprises transporting the liquid through the supply lumen at a first flow rate and ejecting the liquid from the supply lumen into the catheter body, wherein the catheter body comprises another liquid flowing at a second flow rate higher than the first flow rate.

8. The method of claim 1, further comprising irrigating the anatomical structure via an irrigation lumen in fluid communication with a second liquid source.

9. The method of claim 8, wherein the second liquid source provides a gravity fed pressure to the irrigation lumen.

10. The method of claim 1, further comprising controlling a fluid inflow and a fluid outflow of the anatomical structure based on at least one of a sensed pressure, a sensed flow rate, or a sensed temperature.

11. The method of claim 1, wherein the liquid is ejected into the aspiration lumen along a helical path within the catheter body.

12. A method of removing a biological object from an anatomical structure, the method comprising:
creating a vacuum with a catheter in the anatomical structure, the catheter including a proximal end and a distal end, an aspiration lumen positioned in a catheter body, and a supply lumen positioned in the aspiration lumen, the vacuum being created at one or more distal openings in the catheter body to attract a biological object toward the one or more distal openings by ejecting a liquid from an end of the supply lumen into the aspiration lumen;
breaking the biological object attracted to the one or more distal openings in the catheter body into a plurality of fragments; and
aspirating at least some of the plurality of fragments of the biological object through the one or more distal openings in the catheter body and transporting at least some of the plurality of fragments of the biological object along the aspiration lumen toward the proximal end of the catheter.

13. The method of claim 14, wherein aspirating at least some of the plurality of fragments of the biological object through one or more of the plurality of vent port openings improves visibility of the anatomical structure.

14. The method of claim 12, further comprising ejecting the liquid from the catheter body through one or more of a plurality of vent port openings formed in the catheter body.

15. The method of claim 12, wherein the liquid is ejected from the distal end of the supply lumen into the aspiration lumen.

16. The method of claim 12, wherein ejecting the liquid comprises ejecting the liquid at least partially toward the proximal end of the catheter body.

17. The method of claim 12, further comprising applying negative pressure to the proximal end of the catheter to aspirate at least some of the plurality of fragments of the biological object.

18. The method of claim 12, further comprising irrigating the anatomical structure via an irrigation lumen in fluid communication with a second liquid source.

19. The method of claim 18, wherein the second liquid source provides a gravity fed pressure to the irrigation lumen.

20. The method of claim 12, further comprising controlling a fluid inflow and a fluid outflow of the anatomical structure based on at least one of a sensed pressure, a sensed flow rate, or a sensed temperature.

21. The method of claim 12, further comprising, with a reciprocator, axially moving an ablation instrument positioned within the aspiration lumen.

22. The method of claim 12, wherein a portion of the distal end of the catheter body is transparent at wavelengths corresponding to visible light and wavelengths corresponding to laser energy emitted by an ablation instrument.
